(19) United States
(12) Patent Application Publication
(10) Pub. No.: US 2005/0123940 A1
(43) Pub. Date: Jun. 9, 2005

(54) Compositions and methods for synthesizing cDNA

(75) Inventors: Joseph A. Sorge, Del Mar, CA (US); Holly Hogrefe, San Diego, CA (US); Connie Jo Hansen, San Diego, CA (US); Bahram Arezi, Carlsbad, CA (US); Rebecca Lynn Mullinax, San Diego, CA (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 10/853,973

(22) Filed: May 26, 2004

Related U.S. Application Data
(63) Continuation-in-part of application No. 10/435,766, filed on May 12, 2003.
Application No. 10/435,766 is a continuation-in-part of application No. 10/223,650, filed on Aug. 19, 2002.
Application No. 10/223,650 is a continuation-in-part of application No. 09/896,923, filed on Jun. 29, 2001.
Application No. 09/896,923 is a continuation-in-part of application No. 09/698,341, filed on Oct. 27, 2000.
(60) Provisional application No. 60/162,600, filed on Oct. 29, 1999.

(30) Foreign Application Priority Data
Oct. 27, 2000 (WO) PCT/US00/29706

(51) Int. Cl. C12Q 1/68; C12P 19/34; C12N 9/22

(52) U.S. Cl. 435/6; 435/91.2; 435/199

(57) ABSTRACT

The present invention relates to composition, kits and methods comprising a mutant DNA polymerase exhibiting increased reverse transcriptase activity. The invention also relates to methods of generating modified cDNA.

### RELATED APPLICATIONS

This application is a Continuation-in-Part of U.S. application Ser. No. 10/435,766, filed May 12, 2003, which is a Continuation-in Part of U.S. application Ser. No. 10/223,650, filed Aug. 19, 2002, which is a Continuation-in-Part of U.S. application Ser. No. 09/896,923, filed Jun. 29, 2001, which is a Continuation-in-Part of U.S. Utility application Ser. No. 09/698,341, filed Oct. 27, 2000, which claims the priority of U.S. Provisional Application No. 60/162,600, filed Oct. 29, 1999. This application also claims the priority of International Application No. PCT/US00/29706, filed Oct. 27, 2000. Each of these applications is incorporated herein by reference in their entirety, including figures and drawings.

### FIELD OF THE INVENTION

The present invention relates to compositions, kits and methods utilizing DNA polymerase enzymes exhibiting an increased reverse transcriptase activity. The enzymes of the invention are useful in many applications calling for the detectable labeling of nucleic acids.

### BACKGROUND

Reverse transcription (RT) and the polymerase chain reaction (PCR) are critical to many molecular biology and related applications, particularly to gene expression analysis applications. Reverse transcription is commonly performed with viral reverse transcriptase isolated from Avian myeloblastosis virus (AMV-RT) or Moloney murine leukemia virus (MMLV-RT), which are active in the presence of magnesium ions. Reverse transcription is useful in the detectable labeling of nucleic acids. Detectable labeling is required for many applications in molecular biology, including applications for research as well as clinical diagnostic techniques. A commonly used method of labeling nucleic acids uses one or more non-conventional nucleotides and a polymerase enzyme that catalyzes the template-dependent incorporation of the non-conventional nucleotide(s) into the newly synthesized complementary strand.

Reverse transcription is also used to prepare template DNA (e.g., cDNA) from an initial RNA sample (e.g. mRNA), which template DNA is then amplified using PCR to produce a sufficient amount of amplified product for the application of interest.

The RT and PCR steps of DNA amplification can be carried out as a two-step or one-step process.

In one type of two-step process, the first step involves synthesis of first strand cDNA with a reverse transcriptase, following by a second PCR step. In certain protocols, these steps are carried out in separate reaction tubes. In these two tube protocols, following reverse transcription of the initial RNA template in the first tube, an aliquot of the resultant product is then placed into the second PCR tube and subjected to PCR amplification.

In a second type of two-step process, both RT and PCR are carried out in the same tube using a compatible RT and PCR buffer. Typically, reverse transcription is carried out first, followed by addition of PCR reagents to the reaction tube and subsequent PCR.

A variety of one-step RT-PCR protocols have been developed, see Blain & Goff, J. Biol. Chem. (1993) δ: 23585-23592; Blain & Goff, J. Virol. (1995) 69:4440-4452; Sellner et al., J. Virol. Method. (1994) 49:47-58; PCR, Essential Techniques (ed. J. F. Burke, J. Wiley & Sons, New York) (1996) pp61-63; 80-81.

Some one-step systems are commercially available, for example, SuperScript One-Step RT-PCR System description on the world-wide web at lifetech.com/world_whatsnew/archive/nz₁₋₋₃.html; Access RT-PCR System and Access RT-PCR Introductory System described on the world wide web at promega.com/tbs/tb220/tb220.html; AdvanTaq & AdvanTaq Plus PCR kits and User Manual available at www.clontech.com, and ProSTAR™ HF single-tube RT-PCR kit (Stratagene, Catalog No. 600164, information available on the world wide web at stratagene.com).

Certain RT-PCR methods use an enzyme blend or enzymes with both reverse transcriptase and DNA polymerase or exonuclease activities, e.g., as described in U.S. Pat. Nos. 6,468,775; 6,399,320; 5,310,652; 6,300,073; Patent Application No. U.S. 2002/0119465A1; EP 1,132,470A1 and WO 00/71739A1, all of which are incorporated herein by reference.

Some existing RT-PCR one-step methods utilize the native reverse transcriptase activity of DNA polymerases of thermophilic organisms which are active at higher temperatures, for example, as described in the references cited above herein, and in U.S. Pat. Nos. 5,310,652, 6,399,320, 5,322,770, and 6,436677; Myers and Gelfand, 1991, Biochem., 30:7661-7666; all of which are incorporated herein by reference. Thermostable DNA polymerases with reverse transcriptase activities are commonly isolated from Thermus species.

There is a need in the art for DNA polymerases exhibiting increased reverse transcriptase activity. There is particularly a need in the art for thermostable DNA polymerases exhibiting increased reverse transcriptase activity that are able to incorporate non-conventional nucleotides in order to generate a nucleic acid probe.

Recently, U.S. Patent Application 2002/0012970 (incorporated herein by reference) describes modifying a thermostable DNA polymerase to obtain RT activity for combined RT-PCR reaction.

### SUMMARY OF THE INVENTION

It is an object of the present invention to provide kits, compositions and methods utilizing DNA polymerase enzymes exhibiting an increased reverse transcriptase activity. Furthermore, it is an object of the invention to provide kits, compositions and methods for generating a modified nucleic acid. Enzymes of the present invention are useful in many applications calling for the detectable labeling of nucleic acids.

In a first aspect, a composition is disclosed comprising a mutant Family B DNA polymerase and at least one amino allyl modified nucleotide, wherein the mutant exhibits an increased reverse transcriptase activity.

In one embodiment, the mutant Family B DNA polymerase is a mutant of a wild-type Family B DNA polymerase that has an LYP motif in Region II at a position corresponding to L409 of Pfu DNA polymerase.

In another embodiment of the composition, the mutant Family B DNA polymerase is the mutant of a wild type DNA polymerase selected from the group consisting of a Pfu DNA polymerase and JDF-3 DNA polymerase.

In another embodiment of the composition, the mutant Family B DNA polymerase is the mutant of a wild-type polymerase comprising an amino acid sequence selected from the group consisting of SEQ ID Nos. 1, 3, 5, 7, 9, 11, 13, 15, 17, 19, 21, and 23.

In another embodiment of the composition the mutant Family B DNA polymerase comprises an amino acid mutation at the amino acids corresponding to L409 to P411 of SEQ ID NO:3.

In another embodiment, the mutant Family B DNA polymerase comprises an amino acid mutation at the amino acid corresponding to L409 of SEQ ID NO: 3.

In another embodiment, the amino acid mutation at the amino acid corresponding to L409 of SEQ ID NO: 3 is a leucine to phenylalanine mutation, leucine to tyrosine mutation, leucine to histidine mutation or a leucine to tryptophan mutation.

In another embodiment of the composition, the mutant Family B DNA polymerase further exhibits a decreased 3′-5′ exonuclease activity.

In another embodiment the mutant Family B DNA polymerase further exhibits a reduced base analog detection activity.

In another embodiment, the mutant DNA polymerase further exhibits a decreased 3′-5′ exonuclease activity and a reduced base analog detection activity.

In another embodiment, the composition further comprises one or more reagents selected from the group consisting of: reaction buffer, dNTP, and control primers.

In a further embodiment the dNTP of the composition comprises an additional non-conventional nucleotide.

In still a further embodiment, the non-conventional nucleotides are selected from the group consisting of: dideoxynucleotides, ribonucleotides, amino allyl modified nucleotides and conjugated nucleotides.

In still a further embodiment, the conjugated nucleotides are selected from the group consisting of radiolabeled nucleotides, fluorescently labeled nucleotides, biotin labeled nucleotides, chemiluminescently labeled nucleotides and quantum dot labeled nucleotides.

In another embodiment, the composition further comprises one or more reagents selected from the group consisting of: formamide, DMSO, betaine, trehalose, low molecular weight amides, sulfones, a Family B accessory factor, a single stranded DNA binding protein, a DNA polymerase other than the mutant Family B DNA polymerase, another reverse transcriptase enzyme, an RNA polymerase and an exonuclease.

In another aspect, a kit is disclosed comprising a mutant Family B DNA polymerase, at least one amino allyl modified nucleotide, and packaging materials therefor. The mutant Family B DNA polymerase exhibits an increased reverse transcriptase activity.

In a further embodiment the amino allyl modified nucleotide is amino allyl dUTP, amino allyl UTP or amino allyl dCTP.

In one embodiment of the kit, the mutant Family B DNA polymerase is a mutant of a wild-type Family B DNA polymerase that has an LYP motif in Region II at a position corresponding to L409 of Pfu DNA polymerase.

In another embodiment, the wild-type Family B DNA polymerase comprises an amino acid sequence selected from SEQ ID Nos. 1, 3, 5, 7, 9, 11, 13, 15, 17, 19, 21, and 23.

In another embodiment of the kit, the mutant Family B DNA polymerase is the mutant of a wild type DNA polymerase selected from the group consisting of a Pfu DNA polymerase and JDF-3 DNA polymerase.

In another embodiment of the composition the mutant Family B DNA polymerase comprises an amino acid mutation at the amino acids corresponding to L409 to P411 of SEQ ID NO:3.

In another embodiment, the mutant Family B DNA polymerase comprise an amino acid mutation at the position corresponding to L409 of SEQ ID NO: 3.

In another embodiment, the amino acid mutation at the amino acid corresponding to L409 of SEQ ID NO: 3 is a leucine to phenylalanine mutation, leucine to tyrosine mutation, leucine to histidine mutation or a leucine to tryptophan mutation.

In another embodiment of the kit, the mutant Family B DNA polymerase further exhibits a decreased 3′-5′ exonuclease activity.

In another embodiment the mutant Family B DNA polymerase further exhibits a reduced base analog detection activity.

In another embodiment, the mutant DNA polymerase further exhibits a decreased 3′-5′ exonuclease activity and a reduced base analog detection activity.

In another embodiment, the kit further comprises one or more reagents selected from the group consisting of: reaction buffer, dNTP, and a control primer.

In a further embodiment of the kit, the dNTP comprises an additional non-conventional nucleotide.

In still a further embodiment, the non-conventional nucleotides are selected from the group consisting of: dideoxynucleotides, ribonucleotides, amino allyl modified nucleotides and conjugated nucleotides.

In still a further embodiment, the conjugated nucleotides are selected from the group consisting of radiolabeled nucleotides, fluorescently labeled nucleotides, biotin labeled nucleotides, chemiluminescently labeled nucleotides and quantum dot labeled nucleotides.

In another embodiment, the kit further comprises one or more reagents selected from the group consisting of: formamide, DMSO, betaine, trehalose, low molecular weight amides, sulfones, an Family B accessory factor, a single-stranded DNA binding protein, a DNA polymerase other than the mutant Family B DNA polymerase, another reverse transcriptase enzyme, an RNA polymerase and an exonuclease.

In another aspect, a method for reverse transcribing an RNA template is disclosed, comprising incubating the RNA template in a reaction mixture comprising a mutant Family B DNA polymerase and an amino allyl modified nucleotide. The mutant Family B DNA polymerase exhibits an increased reverse transcriptase activity.

In a further embodiment the amino allyl modified nucleotide is amino allyl dUTP, amino allyl UTP or amino allyl dCTP.

In one embodiment, the mutant Family B DNA polymerase is a mutant of the wild-type Family B DNA polymerase that has an LYP motif in Region II at a position corresponding to L409 of Pfu DNA polymerase.

In another embodiment of the method the mutant Family B DNA polymerase is the mutant of a wild type DNA polymerase selected from the group consisting of a Pfu DNA polymerase and JDF-3 DNA polymerase.

In another embodiment, the wild-type Family B DNA polymerase comprises an amino acid sequence selected from SEQ ID Nos. 1, 3, 5, 7, 9, 11, 13, 15, 17, 19, 21 and 23.

In another embodiment of the method the mutant Family B DNA polymerase comprises an amino acid mutation at the amino acids corresponding to L409 to P411 of SEQ ID NO:3.

In another embodiment of the method, the mutant Family B DNA polymerase comprise an amino acid mutation at the position corresponding to L409 of SEQ ID NO: 3.

In further embodiment, the amino acid mutation at the amino acid corresponding to L409 of SEQ ID NO: 3 is a leucine to phenylalanine mutation, leucine to tyrosine mutation, leucine to histidine mutation or a leucine to tryptophan mutation.

In another embodiment of the method, the mutant Family B DNA polymerase further exhibits a decreased 3′-5′ exonuclease activity.

In another embodiment the mutant Family B DNA polymerase further exhibits a reduced base analog detection activity.

In another embodiment, the mutant DNA polymerase further exhibits a decreased 3′-5′ exonuclease activity and a reduced base analog detection activity.

In another aspect, a method for generating modified complementary strand of DNA is disclosed wherein one combines a template RNA molecule with a mutant Family B DNA polymerase, exhibiting an increased reverse transcriptase activity, in a reaction mixture comprising at least one non-conventional nucleotide, under conditions and for a time sufficient to permit the mutant Family B DNA polymerase to synthesize a complementary DNA stand incorporating the non-conventional nucleotide into the synthesized complementary DNA stand.

In one embodiment, the mutant Family B DNA polymerase is a mutant of the wild-type Family B DNA polymerase that has an LYP motif in Region II at a position corresponding to L409 of Pfu DNA polymerase.

In another embodiment of the method the mutant Family B DNA polymerase is the mutant of a wild type DNA polymerase selected from the group consisting of a Pfu DNA polymerase and JDF-3 DNA polymerase.

In another embodiment, the wild-type Family B DNA polymerase comprises an amino acid sequence selected from SEQ ID Nos. 1, 3, 5, 7, 9, 11, 13, 15, 17, 19, 21 and 23.

In another embodiment of the method the mutant Family B DNA polymerase comprises an amino acid mutation at the amino acids corresponding to L409 to P411 of SEQ ID NO:3.

In another embodiment, the mutant Family B DNA polymerase comprises an amino acid mutation at the position corresponding to L409 of SEQ ID NO: 3.

In another embodiment, the amino acid mutation at the amino acid corresponding to L409 of SEQ ID NO: 3 is a leucine to phenylalanine mutation, leucine to tyrosine mutation, leucine to histidine mutation or a leucine to tryptophan mutation.

In another embodiment of the method, the mutant Family B DNA polymerase further exhibits a decreased 3′-5′ exonuclease activity.

In another embodiment the mutant Family B DNA polymerase further exhibits a reduced base analog detection activity.

In another embodiment, the mutant DNA polymerase further exhibits a decreased 3′-5′ exonuclease activity and a reduced base analog detection activity.

In another embodiment, the non-conventional nucleotide is selected from the group consisting of: dideoxynucleotides, ribonucleotides, amino allyl modified nucleotides and conjugated nucleotides.

In a further embodiment, the conjugated nucleotides are selected from the group consisting of radiolabeled nucleotides, fluorescently labeled nucleotides, biotin labeled nucleotides, chemiluminescently labeled nucleotides and quantum dot labeled nucleotides.

In a further embodiment, the method of generating a modified cDNA further comprises a coupling step.

In yet a further embodiment, the coupling step comprising coupling the modified cDNA to a fluorescent dye containing a NHS- or STP-ester.

In another aspect a method for amplifying an RNA molecule is disclosed, the method comprising incubating a template RNA molecule with a first primer complex in a first reaction mixture comprising a mutant Family B DNA polymerase exhibiting an increased reverse transcriptase activity and wherein the incubation permits the synthesis of a complementary DNA template and wherein the primer complex comprises a primer complementary to the target sequence and promoter region. Incubating the complementary DNA template and a second primer complex in a second reaction mixture wherein second reaction mixture permits synthesis of a second complementary DNA containing the promoter region. The final step involving transcribing copies of RNA initiated from the promoter region of the primer complex and therefore generating anti-sense RNA.

In one embodiment, the recombinant Family B DNA polymerase is a mutant of the wild-type Family B DNA polymerase that has an LYP motif in Region II at a position corresponding to L409 of Pfu DNA polymerase.

In another embodiment, the mutant Family B DNA polymerase is the mutant of a wild type DNA polymerase selected from the group consisting of a Pfu DNA polymerase and JDF-3 DNA polymerase.

In another embodiment, the wild-type Family B DNA polymerase comprises an amino acid sequence selected from SEQ ID Nos. 1, 3, 5, 7, 9, 11, 13, 15, 17, 19, 21 and 23.

In another embodiment, the mutant Family B DNA polymerase comprises an amino acid mutation at the amino acids corresponding to L409 to P411 of SEQ ID NO:3.

In another embodiment, the Family B DNA polymerase comprises an amino acid mutation at the position corresponding to L409 of SEQ ID NO: 3.

In another embodiment, the amino acid mutation at the amino acid corresponding to L409 of SEQ ID NO: 3 is a leucine to phenylalanine mutation, leucine to tyrosine mutation, leucine to histidine mutation or a leucine to tryptophan mutation.

In another embodiment, the mutant Family B DNA polymerase further exhibits a decreased 3′-5′ exonuclease activity.

In another embodiment the mutant Family B DNA polymerase further exhibits a reduced base analog detection activity.

In another embodiment, the mutant DNA polymerase further exhibits a decreased 3′-5′ exonuclease activity and a reduced base analog detection activity.

In another embodiment of the method the first and second reaction mixtures are conducted in the same reaction tube.

In one embodiment, the second reaction mixture comprises a second DNA polymerase or a combination of two or more other DNA polymerases.

In another embodiment, the second DNA polymerase is a wild-type DNA polymerase.

In another embodiment, the second DNA polymerase comprises Taq DNA polymerase, Pfu Turbo DNA polymerase Klenow, E Coli DNA pol I, Exo⁻ Pfu V93, Exo⁻ Pfu or a combination of these.

In a further embodiment of the method, the transcribing step incorporates a non-conventional nucleotide into the anti-sense RNA.

In a further embodiment of the method, the transcription reaction is followed by a coupling step.

In yet a further embodiment, the coupling step comprising coupling the modified RNA to a fluorescent dye containing a NHS- or STP-ester leaving group.

In a final aspect of the invention, a method for amplifying an RNA molecule is disclosed, comprising incubating a template RNA molecule with a first primer complex in a first reaction mixture comprising a mutant Family B DNA polymerase exhibiting an increased reverse transcriptase activity, wherein the incubation permits synthesis of a complementary DNA template. Incubating the complementary DNA template and a second primer complex in a second reaction mixture, wherein the second primer complex comprises a primer complementary to the template and a promoter region and wherein the second reaction mixture permits synthesis of a second complementary DNA containing the promoter region. In a final step transcribing copies of RNA initiated from the promoter region of the second primer complex and generating synthesized RNA.

In one embodiment of the invention the mutant Family B DNA polymerase is the mutant of the wild-type Family B DNA polymerase that has an LYP motif in Region II at a position corresponding to L409 of Pfu DNA polymerase. In another embodiment of the invention, the mutant Family B DNA polymerase is the mutant of a wild type DNA polymerase selected from the group consisting of a Pfu DNA polymerase and JDF-3 DNA polymerase.

In another embodiment of the invention, the mutant Family B DNA polymerase is a mutant of the wild-type Family B DNA polymerase comprising an amino acid sequence selected from the group consisting of SEQ ID Nos. 1, 3, 5, 7, 9, 11, 13, 15, 17, 19, 21 and 23.

In another embodiment of the invention, the mutant Family B DNA polymerase comprises an amino acid mutation at the amino acids corresponding to L409 to P411 of SEQ ID NO:3.

In another embodiment of the invention, the mutant Family B DNA polymerase comprises an amino acid mutation at the position corresponding to L409 of SEQ ID NO:3.

In another embodiment, the mutant Family B DNA polymerase further exhibits a decreased 3′-5′ exonuclease activity.

In another embodiment the mutant Family B DNA polymerase further exhibits a reduced base analog detection activity.

In another embodiment, the mutant DNA polymerase further exhibits a decreased 3′-5′ exonuclease activity and a reduced base analog detection activity.

In a further embodiment of the invention, the amino acid mutation at the amino acid corresponding to L409 of SEQ ID NO: 3 is a leucine to phenylalanine mutation, leucine to tyrosine mutation, leucine to histidine mutation or a leucine to tryptophan mutation.

In another embodiment of the invention, the first and second reaction mixtures occur in the same reaction tube.

In another embodiment of the invention, the second reaction mixture comprises a second DNA polymerase or a combination of two or more other DNA polymerases.

In another embodiment of the invention, the second DNA polymerase is a wild-type DNA polymerase.

In another embodiment of the invention, the second DNA polymerase comprises Taq DNA polymerase, Pfu Turbo DNA polymerase, Klenow, E coli DNA pol I, Exo− Pfu V93, and Exo− Pfu.

In another embodiment of the invention, the first primer and the second primer complexes are the same.

In another embodiment of the invention, the primer complexes comprise a primer complementary to the target sequence and a promoter region.

In a further embodiment of the method, the transcribing step incorporates a non-conventional nucleotide into the synthesized RNA.

In a further embodiment of the method, the transcription reaction is followed by a coupling step.

In a final embodiment, the coupling step comprising coupling the synthesized RNA to a fluorescent dye containing a NHS- or STP-ester leaving group.

In a final embodiment, the first or second primer complex contains a non-conventional nucleotide.

### BRIEF DESCRIPTION OF THE DRAWINGS

FIG. 1 shows the primer sequences used for Pfu or JDF-3 mutagenesis (SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39) according to some embodiments of the present invention.

FIG. 2 shows a comparison of RNA dependent DNA polymerization (reverse-transcriptase, RT) activity and DNA dependent DNA polymerase (DNA polymerase) activity in clarified lysates of wild-type and mutant Pfu and JDF-3 DNA polymerases. Three different volumes of clarified lysate were used for each polymerase. Top panel, DNA dependent DNA polymerase activity, measured as cpm of ³H-TTP incorporated; middle panel, RNA dependent DNA polymerase activity, measured as cpm of ³H-TTP incorporated; and bottom panel, ratios of RNA dependent polymerase activity over DNA polymerase activity from the samples with 0.2 μl of clarified lysate.

FIG. 3 shows a comparison of RNA dependent DNA polymerase activity and DNA dependent DNA polymerase activity in clarified lysates of Exo+ wild-type and mutant Pfu and JDF-3 DNA polymerases. Three different volumes of clarified lysate were used for each polymerase. Top panel, DNA dependent DNA polymerase activity, measured as cpm of ³H-TTP incorporated; middle panel, RNA dependent DNA polymerase activity, measured as cpm of ³H-TTP incorporated; and bottom panel, ratios of RNA dependent polymerase activity over DNA polymerase activity from the samples with 0.2 μl of clarified lysate.

FIG. 4 shows the results of experiments evaluating the reverse transcriptase activity of purified mutant polymerases according to several embodiments of the invention. Reactions were performed with purified preparations of exo− JDF-3 L408H and L408F mutants and with wild-type JDF-3 and Pfu and RNaseH⁻ MMLV-RT (Stratascript™, Stratagene). Activity is measured as cpm of ³³P-dGTP incorporated. Improved RNA dependent DNA polymerase activity with the mutant polymerases is evident compared to wild type JDF-3 and Pfu.

FIG. 5 shows the results of an experiment evaluating the RNA dependent DNA polymerase activity of purified polymerase mutants by RT-PCR. A different purified polymerase (2 units) was used for each RT reaction, and Taq polymerase was used for subsequent PCR amplification. Products were separated by agarose gel electrophoresis and stained with ethidium bromide. Lane 1, negative control (no RTase); Lane 2, positive control using StrataScript™ RTase (RNaseH⁻ MMLV-RT); Lane 3, exo⁻ JDF-3 polymerase; Lane 4, exo⁻ JDF-3 L408H polymerase; and Lane 5, exo− JDF-3 L408F polymerase.

FIG. 6 is a sequence alignment of several Family B DNA polymerases. Pfu, Pyrococcus furiosus(SEQ ID NO:40; SEQ ID NO:41; SEQ ID NO:42; SEQ ID NO:43; SEQ ID NO:44; SEQ ID NO:45); JDF-3 (SEQ ID NO:46; SEQ ID NO:47; SEQ ID NO:48; SEQ ID NO:49; SEQ ID NO:50; SEQ ID NO:51); Tgo, Thermococcus gorgonarius (SEQ ID NO:52; SEQ ID NO:53; SEQ ID NO:54; SEQ ID NO:55; SEQ ID NO:56; SEQ ID NO:57); Tli, Thermococcus litoralis (SEQ ID NO:58; SEQ ID NO:59; SEQ ID NO:60; SEQ ID NO:61; SEQ ID NO:62; SEQ ID NO:63); Tsp, Thermococcus sp. (SEQ ID NO:64; SEQ ID NO:65; SEQ ID NO:66; SEQ ID NO:67; SEQ ID NO:68; SEQ ID NO:69); Mvo, Methanococcus voltae (SEQ ID NO:70; SEQ ID NO:71; SEQ ID NO:72; SEQ ID NO:73; SEQ ID NO:74; SEQ ID NO:75); RB69, bacteriophage RB69 ((SEQ ID NO:76; SEQ ID NO:77; SEQ ID NO:78; SEQ ID NO:79; SEQ ID NO:80; SEQ ID NO:81); T4, bacteriophage T4 (SEQ ID NO:82; SEQ ID NO:83; SEQ ID NO:84; SEQ ID NO:85; SEQ ID NO:86; SEQ ID NO:87); Eco, Eschericia coli (SEQ ID NO:88; SEQ ID NO:89; SEQ ID NO:90; SEQ ID NO:91; SEQ ID NO:92; SEQ ID NO:93). DNA polymerase sequences from additional species are aligned in Hopfner et al., 1999, Proc. Natl. Acad. Sci. U.S.A. 96: 3600-3605, which is incorporated herein by reference.

FIG. 7 contains the wild-type amino acid and polynucleotide sequences of representative Family B DNA polymerases, including JDF-3 DNA polymerase (SEQ ID NO: 1 and 2, respectively); amino acid sequence in the processed polypeptide is shown in italics SEQ ID NO:103), amino acids targeted for mutation according to several embodiments of the invention are underlined), wild type Pfu DNA polymerase (SEQ ID NO: 3 and 4, respectively), wild type KOD polymerase (SEQ ID NO: 5 and 6, respectively), wild type Vent™ polymerase (SEQ ID NO: 7 and 8, respectively), wild-type Deep Vent polymerase (SEQ ID NO: 9 and 10, respectively), Tgo DNA polymerase (SEQ ID NO: 11 and 12, respectively), Thest Thermococcus strain TY DNA polymerase (SEQ ID NO: 13 and 14, respectively), 9oN Thermococcus species DNA polymerase (SEQ ID NO: 15 and 16, respectively). Methanobacterium thermoautotrophicum DNA polymerase (SEQ ID NO: 17 and 18, respectively), Thermoplasma acidophilum DNA polymerase (SEQ ID NO: 19 and 20, respectively), Pyrobaculum islandicum DNA polymerase (SEQ ID NO:21 and 22, respectively), and the amino acid sequence for Methanococcus jannaschii DNA polymerase (SEQ ID NO: 23).

FIG. 8 shows data from an experiment evaluating the effect of DMSO concentration on the reverse transcriptase activity of the exo+ Pful409Y DNA polymerase mutant. M=RNA size markers. Lanes marked 0-25 correspond to reactions run in the presence of 0-25% DMSO.

FIG. 9 shows data from an experiment evaluating the incorporation of unmodified and amino allyl modified dUTP and dCTP with PfuL409Y or STRATASCRIPT DNA polymerase (Stratagene, La Jolla, Calif.). Results were analyzed on a 1% alkaline agarose gel stained with ethidium bromide. Lane 1, 1 kb DNA ladder; Lane 2, unmodified dNTP; Lane 3, 0.53 mM amino allyl dUTP:0.27 mM dTTP; Lane 4, 0.53 mM amino allyl dCTP:0.27 mM dCTP; Lane 5, 0.265 mM amino allyl dUTP:0.135 mM dTTP and 0.265 mM amino allyl dCTP:0.135 mM dCTP; Lane 6, FAIRPLAY microarray labeling kit (Stratagene, La Jolla, Calif.) with STRATASCRIPT DNA polymerase (Stratagene, La Jolla, Calif.) and amino allyl dUTP.

FIG. 10 shows data from an experiment evaluating the incorporation of amino allyl modified nucleotides by Pfu L409Y or STRATASCRIPT DNA polymerase (Stratagene, La Jolla, Calif.) followed by coupling to Cy5. Results were analyzed on a non-denaturing gel measuring Cy5 fluorescence. Lane 1, 1 kb DNA ladder; Lane 2, unmodified dNTP; Lane 3, 0.53 mM amino allyl dUTP:0.27 mM dCTP; Lane 4, 0.53 mM amino allyl dCTP:0.27 mM dCTP; Lane 5, 0.265 mM amino allyl dUTP:0.135 mM dTTP and 0.265 mM amino allyl dCTP:0.135 mM dCTP; Lane 6, FAIRPLAY microarray labeling kit (Stratagene, La Jolla, Calif.) with STRATASCRIPT DNA polymerase (Stratagene, La Jolla, Calif.) and amino allyl dNTP.

### DETAILED DESCRIPTION

Definitions

As used herein, “polynucleotide polymerase” refers to an enzyme that catalyzes the polymerization of nucleotides, e.g., to synthesize polynucleotide strands from ribonucleoside triphosphates or deoxynucleoside triphosphates. Generally, the enzyme will initiate synthesis at the 3′-end of a primer annealed to a polynucleotide template sequence, and will proceed toward the 5′ end of the template strand. “DNA polymerase” catalyzes the polymerization of deoxynucleotides to synthesize DNA, while “RNA polymerase” catalyzes the polymerization of ribonucleotides to synthesize RNA.

The term “DNA polymerase” refers to a DNA polymerase which synthesizes new DNA strands by the incorporation of deoxynucleoside triphosphates in a template dependent manner. The measurement of DNA polymerase activity may be performed according to assays known in the art, for example, as described by a previously published method (Hogrefe, H. H., et al (01) Methods in Enzymology, 343:91-116). A “DNA polymerase” may be DNA-dependent (i.e., using a DNA template) or RNA-dependent (i.e., using a RNA template).

As used herein, the term “template dependent manner” refers to a process that involves the template dependent extension of a primer molecule (e.g., DNA synthesis by DNA polymerase). The term “template dependent manner” refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)).

As used herein, “thermostable” refers to a property of an enzyme that is active at elevated temperatures and is resistant to DNA duplex-denaturing temperatures in the range of about 93° C. to about 97° C. “Active” means the enzyme retains the ability to effect primer extension reactions when subjected to elevated or denaturing temperatures for the time necessary to effect denaturation of double-stranded nucleic acids. Elevated temperatures as used herein refer to the range of about 70° C. to about 75° C., whereas non-elevated temperatures as used herein refer to the range of about 35° C. to about 50° C.

As used herein, “Archaeal” refers to an organism or to a DNA polymerase from an organism of the kingdom Archaea, e.g., Archaebacteria. An “Archaeal DNA polymerase” refers to any identified or unidentified “Archaeal DNA polymerase,” e.g., as described in Table II under the subheading Archaeal DNA polymerase and Table III, isolated from an Archaeabacteria, e.g., as described in Table IV.

As used herein, “Family B DNA polymerase” refers to any DNA polymerase that is classified as a member of the Family B DNA polymerases, where the Family B classification is based on structural similarity to E. coli DNA polymerase II. Archaeal DNA polymerases are members of the Family B DNA polymerases. The Family B DNA polymerases, formerly known as α-family polymerases, include, but are not limited to those listed as such in Tables I-III.

As used herein, the term “reverse transcriptase (RT)” describes a class of polymerases characterized as RNA dependent DNA polymerases. RT is a critical enzyme responsible for the synthesis of cDNA from viral RNA for all retroviruses, including HIV, HTLV-I, HTLV-II, FeLV, FIV, SIV, AMV, MMTV, and MoMuLV. For review, see e.g. Levin, 1997, Cell, 88:5-8; Brosius et al., 1995, Virus Genes 11:163-79. Known reverse transcriptases from viruses require a primer to synthesize a DNA transcript from an RNA template. Reverse transcriptase has been used primarily to transcribe RNA into cDNA, which can then be cloned into a vector for further manipulation or used in various amplification methods such as polymerase chain reaction (PCR), nucleic acid sequence-based amplification (NASBA), transcription mediated amplification (TMA), or self-sustained sequence replication (3SR).

As used herein, the terms “reverse transcription activity” and “reverse transcriptase activity” are used interchangeably to refer to the ability of an enzyme (e.g., a reverse transcriptase or a DNA polymerase) to synthesize a DNA strand (i.e., cDNA) utilizing an RNA strand as a template. Methods for measuring RT activity are provided in the examples herein below and also are well known in the art. For example, the Quan-T-RT assay system is commercially available from Amersham (Arlington Heights, Ill.) and is described in Bosworth, et al., Nature 1989, 341:167-168.

As used herein, the term “increased reverse transcriptase activity” refers to the level of reverse transcriptase activity of a mutant enzyme (e.g., a DNA polymerase) as compared to its wild-type form. A mutant enzyme is said to have an “increased reverse transcriptase activity” if the level of its reverse transcriptase activity (as measured by methods described herein or known in the art) is at least 20% or more than its wild-type form, for example, at least 25%, 30%, 40%, 50%, 60%, 70%, 80%, 90%, 100% more or at least 2-fold, 3-fold, 4-fold, 5-fold, or 10-fold or more.

As used herein, “non-conventional nucleotide” refers to a) a nucleotide structure that is not one of the four conventional deoxynucleotides dATP, dCTP, dGTP, and dTTP recognized by and incorporated by a DNA polymerase, b) a synthetic nucleotide that is not one of the four conventional deoxynucleotides in (a), c) a modified conventional nucleotide, or d) a ribonucleotide (since they are not normally recognized or incorporated by DNA polymerases) and modified forms of a ribonucleotide. Preferably, a “non-conventional nucleotide” is an amino allyl modified nucleotide, e.g., amino allyl dUTP, amino allyl UTP, and amino allyl dCTP.

Non-conventional nucleotides include but are not limited to those listed in Table V, which are commercially available, for example, from New England Nuclear and Sigma-Aldrich. Any one of the above non-conventional nucleotides may be a “conjugated nucleotide”, which as used herein refers to nucleotides bearing a detectable label, including but not limited to a fluorescent label, isotope, chemiluminescent label, quantum dot label, antigen, or affinity moiety.

As used herein, “amino allyl modified nucleotide” refers to a nucleotide that has been modified to contain a primary amine at the 5′-end of the nucleotide, preferably with one or more methylene groups disposed between the primary amine and the nucleic acid portion of the nucleic acid polymer. Six is a preferred number of methylene groups. Amino allyl modified nucleotides can be introduced into nucleic acids by polymerases disclosed herein. “Amino-allyl modified nucleotides” include amino allyl dUTP, amino allyl UTP and amino allyl dCTP.

As used herein, “detectable labeled” refers to a structural modification that incorporates a functional group (label) that can be readily detected by various means. Compounds that can be detectable labeled include but are not limited to nucleotide analogs. Detectable nucleotide analog labels include but are not limited to fluorescent compounds, e.g., Cy5, Cy3 etc., isotopic compounds, chemiluminescent compound, quantum dot labels, biotin, enzymes, electron-dense reagents, and haptens or proteins for which antisera or monoclonal antibodies are available. The various means of detection include but are not limited to spectroscopic, photochemical, biochemical, immunochemical, or chemical means.

As used herein, “modified nucleic acid” refers to a nucleic acid generated by a polynucleotide polymerase, e.g., DNA polymerase, RNA polymerase, reverse transcriptase or a DNA polymerase of the current invention, wherein the “modified nucleic acid” includes at least one non-conventional nucleotide.

As used herein, “exonuclease” refers to an enzyme that cleaves bonds, preferably phosphodiester bonds, between nucleotides one at a time from the end of a DNA molecule. An exonuclease can be specific for the 5′ or 3′ end of a DNA molecule, and is referred to herein as a 5′ to 3′ exonuclease or a 3′ to 5′ exonuclease. The 3′ to 5′ exonuclease degrades DNA by cleaving successive nucleotides from the 3′ end of the polynucleotide while the 5′ to 3′ exonuclease degrades DNA by cleaving successive nucleotides from the 5′ end of the polynucleotide. During the synthesis or amplification of a polynucleotide template, a DNA polymerase with 3′ to 5′ exonuclease activity (3′ to 5′ exo⁺) has the capacity of removing mispaired base (proofreading activity), therefore is less error-prone (i.e., with higher fidelity) than a DNA polymerase without 3′ to 5′ exonuclease activity (3′ to 5′ exo⁻). The exonuclease activity can be measured by methods well known in the art. For example, one unit of exonuclease activity may refer to the amount of enzyme required to cleave 1 μg DNA target in an hour at 37° C.

The term “substantially free of 5′ to 3′ exonuclease activity” indicates that the enzyme has less than about 5% of the 5′ to 3′ exonuclease activity of wild-type enzyme, preferably less than about 3% of the 5′ to 3′ exonuclease activity of wild-type enzyme, and most preferably no detectable 5′ to 3′ exonuclease activity. The term “substantially free of 3′ to 5′ exonuclease activity” indicates that the enzyme has less than about 5% of the 3′ to 5′ exonuclease activity of wild-type enzyme, preferably less than about 3% of the 3′ to 5′ exonuclease activity of wild-type enzyme, and most preferably no detectable 3′ to 5′ exonuclease activity.

The term “fidelity” as used herein refers to the accuracy of DNA polymerization by template-dependent DNA polymerase, e.g., RNA-dependent or DNA-dependent DNA polymerase. The fidelity of a DNA polymerase is measured by the error rate (the frequency of incorporating an inaccurate nucleotide, i.e., a nucleotide that is not incorporated at a template-dependent manner). The accuracy or fidelity of DNA polymerization is maintained by both the polymerase activity and the 3′-5′ exonuclease activity of a DNA polymerase. The term “high fidelity” refers to an error rate of 5×10⁻⁶ per base pair or lower. The fidelity or error rate of a DNA polymerase may be measured using assays known to the art (see for example, Lundburg et al., 1991 Gene, 108:1-6).

As used herein, “reduced base analog detection” refers to a DNA polymerase with a reduced ability to recognize a base analog, for example, uracil or inosine, present in a DNA template. In this context, mutant DNA polymerase with “reduced” base analog detection activity is a DNA polymerase mutant having a base analog detection activity which is lower than that of the wild-type enzyme, i.e., having less than 10% (e.g., less than 8%, 6%, 4%, 2% or less than 1%) of the base analog detection activity of that of the wild-type enzyme. base analog detection activity may be determined according to the assays similar to those described for the detection of DNA polymerases having a reduced uracil detection as described in Greagg et al. (1999) Proc. Natl. Acad. Sci. 96, 9045-9050 and in Example 3 of pending U.S. patent application Ser. No. 10/408,601 (Hogrefe et al; filed Apr. 7, 2003), which is herein incorporated by reference. Alternatively, “reduced” base analog detection refers to a mutant DNA polymerase with a reduced ability to recognize a base analog, the “reduced” recognition of a base analog being evident by an increase in the amount of >10 Kb PCR of at least 10%, preferably 50%, more preferably 90%, most preferably 99% or more, as compared to a wild type DNA polymerase without a reduced base analog detection activity. The amount of a >10 Kb PCR product is measured either by spectrophotometer-absorbance assays of gel eluted >10 Kb PCR DNA product or by fluorometric analysis of >10 Kb PCR products in an ethidium bromide stained agarose electrophoresis gel using, for example, a Molecular Dynamics (MD) FluorImager™ (Amersham Biosciences, catalogue #63-0007-79). DNA polymerases with reduced base analog detection activity are taught in U.S. Ser. No. 10/408,601, herein incorporated by reference in its entirety.

As used herein, “base analogs” refer to bases that have undergone a chemical modification as a result of the elevated temperatures required for PCR reactions. In a preferred embodiment, “base analog” refers to uracil that is generated by deamination of cytosine. In another preferred embodiment, “base analog” refers to inosine that is generated by deamination of adenine.

As used herein, an “amplified product” refers to the single- or double-strand polynucleotide population at the end of an amplification reaction. The amplified product contains the original polynucleotide template and polynucleotide synthesized by DNA polymerase using the polynucleotide template during the amplification reaction.

As used herein, “polynucleotide template” or “target polynucleotide template” refers to a polynucleotide (RNA or DNA) which serves as a template for a DNA polymerase to synthesize DNA in a template-dependent manner. The “amplified region,” as used herein, is a region of a polynucleotide that is to be either synthesized by reverse transcription or amplified by polymerase chain reaction (PCR). For example, an amplified region of a polynucleotide template may reside between two sequences to which two PCR primers are complementary.

As used herein, “primer” refers to an oligonucleotide, whether natural or synthetic, which is substantially complementary to a template DNA or RNA (i.e., at least 7 out of 10, preferably 9 out of 10, more preferably 9 out of 10 bases are fully complementary) and can anneal to a complementary template DNA or RNA to form a duplex between the primer and the template. A primer may serve as a point of initiation of nucleic acid synthesis by a polymerase following annealing to a DNA or RNA strand. A primer is typically a single-stranded oligodeoxyribonucleotide. The appropriate length of a primer depends on the intended use of the primer, typically ranges from about 10 to about 60 nucleotides in length, preferably 15 to 40 nucleotides in length. A primer can include one or more non-conventional nucleotides. As used herein, the term “primer complex” refers to an oligonucleotide having a primer and an RNA polymerase promoter region. The primer component will be capable of acting as a point of initiation of synthesis, typically DNA replication, when placed under conditions in which synthesis of a primer extension product that is complementary to a nucleic acid strand is induced, i.e., in the presence of appropriate nucleotides and a replicating agent (e.g., a DNA polymerase of the current invention) under suitable conditions, which are well known in the art. The RNA polymerase promoter region will be capable of acting as a point of initiation of RNA synthesis when placed under conditions in which synthesis of a primer extension product that is complementary to a nucleic acid strand is induced, i.e., in the presence of appropriate nucleotides and a replicating agent (e.g., an RNA polymerase) under suitable conditions, which are well known in the art.

“Complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide.

As used herein, the term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. “Homology”, with respect to polypeptides (i.e., amino acids), may be determined using a program, such as BLASTP version 2.2.2 with the default parameters, which aligns the polypeptides or fragments being compared and determines the extent of amino acid identity or similarity between them. It will be appreciated that amino acid “homology” includes conservative substitutions, i.e. those that substitute a given amino acid in a polypeptide by another amino acid of similar characteristics. Typically seen as conservative substitutions are the following replacements: replacements of an aliphatic amino acid such as Ala, Val, Leu and Ile with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue.

As used herein in relation to the position of an amino acid mutation, the term “corresponding to” refers to an amino acid in a first polypeptide sequence that aligns with a given amino acid in a reference polypeptide sequence when the first polypeptide and reference polypeptide sequences are aligned. Alignment is performed by one of skill in the art using software designed for this purpose, for example, BLASTP version 2.2.2 with the default parameters for that version. As an example of amino acids that “correspond,” L408 of the JDF-3 Family B DNA polymerase of SEQ ID NO: 1 “corresponds to” L409 of Pfu DNA polymerase, and vice versa, and L409 of Pfu DNA polymerase “corresponds to” L454 of Methanococcus voltae DNA polymerase and vice versa.

The term “wild-type” refers to a gene or gene product which has the characteristics of that gene or gene product when isolated from a naturally occurring source. In contrast, the term “modified” or “mutant” refers to a gene or gene product which displays altered nucleotide or amino acid sequence(s) (i.e., mutations) when compared to the wild-type gene or gene product. For example, a mutant enzyme in the present invention is a mutant DNA polymerase which exhibits an increased reverse transcriptase activity, compared to its wild-type form.

As used herein, the term “mutation” refers to a change in nucleotide or amino acid sequence within a gene or a gene product or outside the gene in a regulatory sequence compared to wild type. The change may be a deletion, substitution, point mutation, mutation of multiple nucleotides or amino acids, transposition, inversion, frame shift, nonsense mutation or other forms of aberration that differentiate the polynucleotide or protein sequence from that of a wild-type sequence of a gene or a gene product.

As used herein, the term “polynucleotide binding protein” refers to a protein which is capable of binding to a polynucleotide. A useful polynucleotide binding protein according to the present invention includes, but is not limited to: Ncp7, recA, SSB, T4gp32, an Family B sequence non-specific double stranded DNA binding protein (e.g., Sso7d, Sac7d, PCNA (WO 01/92501, incorporated herein by reference)), and a helix-hairpin-helix domain.

As used herein, the term “Family B accessory factor” refers to a polypeptide factor that enhances the reverse transcriptase or polymerase activity of a Family B DNA polymerase. The accessory factor can enhance the fidelity and/or processivity of the DNA polymerase or reverse transcriptase activity of the enzyme. Non-limiting examples of Archaeal accessory factors are provided in WO 01/09347, and U.S. Pat. No. 6,333,158 which are incorporated herein by reference.

As used herein, the term “vector” refers to a polynucleotide used for introducing exogenous or endogenous polynucleotide into host cells. A vector comprises a nucleotide sequence which may encode one or more polypeptide molecules. Plasmids, cosmids, viruses and bacteriophages, in a natural state or which have undergone recombinant engineering, are non-limiting examples of commonly used vectors to provide recombinant vectors comprising at least one desired isolated polynucleotide molecule.

As used herein, the term “transformation” or the term “transfection” refers to a variety of art-recognized techniques for introducing exogenous polynucleotide (e.g., DNA) into a cell. A cell is “transformed” or “transfected” when exogenous DNA has been introduced inside the cell membrane. The terms “transformation” and “transfection” and terms derived from each are used interchangeably.

As used herein, an “expression vector” refers to a recombinant expression cassette which has a polynucleotide which encodes a polypeptide (i.e., a protein) that can be transcribed and translated by a cell. The expression vector can be a plasmid, virus, or polynucleotide fragment.

As used herein, “isolated” or “purified” when used in reference to a polynucleotide or a polypeptide means that a naturally occurring nucleotide or amino acid sequence has been removed from its normal cellular environment or is synthesized in a non-natural environment (e.g., artificially synthesized). Thus, an “isolated” or “purified” sequence may be in a cell-free solution or placed in a different cellular environment. The term “purified” does not imply that the nucleotide or amino acid sequence is the only polynucleotide or polypeptide present, but that it is essentially free (about 90-95%, up to 99-100% pure) of non-polynucleotide or polypeptide material naturally associated with it.

As used herein the term “encoding” refers to the inherent property of specific sequences of nucleotides in a polynucleotide, such as a gene in a chromosome or an mRNA, to serve as templates for synthesis of other polymers and macromolecules in biological processes having a defined sequence of nucleotides (i.e., rRNA, tRNA, other RNA molecules) or amino acids and the biological properties resulting therefrom. Thus a gene encodes a protein, if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. A polynucleotide that encodes a protein includes any polynucleotides that have different nucleotide sequences but encode the same amino acid sequence of the protein due to the degeneracy of the genetic code.

Amino acid residues identified herein are preferred in the natural L-configuration. In keeping with standard polypeptide nomenclature, J. Biol. Chem., 243:3557-3559, 1969, abbreviations for amino acid residues are as shown in the following Table I.

TABLE I

| 1-Letter | 3-Letter | AMINO ACID |
|---|---|---|
| Y | Tyr | L-tyrosine |
| G | Gly | glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |

The invention relates to the discovery of thermostable DNA polymerases, e.g., Family B DNA polymerases, that bear one or more mutations resulting in increased reverse transcriptase activity relative to their unmodified wild-type forms. All references described herein are incorporated by reference herein in their entirety.

Thermostable DNA Polymerases

Reverse transcription from many RNA templates by commonly used reverse transcriptases such as avian myeloblastosis virus (AMV) reverse transcriptase and Moloney murine leukemia virus (MMLV) reverse transcriptase is often limited by the secondary structure of the RNA template. Secondary structure in RNA results from hybridization between complementary regions within a given RNA molecule. Secondary structure causes poor synthesis of cDNA and premature termination of cDNA products because polymerases are unable to process through the secondary structure. Therefore, RNAs with secondary structure may be poorly represented in a cDNA library and detection of the presence of RNA with secondary structure in a sample by RT-PCR may be difficult. Furthermore, secondary structure in RNA may cause inconsistent results in techniques such as differential display PCR. Accordingly, it is advantageous to conduct reverse transcription reactions at increased temperatures so that secondary structure is removed or limited.

Several thermostable eubacterial DNA polymerases (e.g., T. thermophilus DNA polymerase, T. aquaticus DNA polymerase (e.g., U.S. Pat. No. 5,322,770), A. thermophilum DNA polymerase (e.g., WO 98/14588), T. vulgaris DNA polymerase (e.g., U.S. Pat. No. 6,436,677), B. caldotenax DNA polymerase (e.g., U.S. Pat. No. 5,436,149); and the polymerase mixture marketed as C. THERM (Boehringer Mannheim) have been demonstrated to possess reverse transcriptase activity. These enzymes can be used at higher temperatures than retroviral reverse transcriptases so that much of the secondary structure of RNA molecules is removed.

The present invention provides a thermostable Family B DNA polymerase with increased reverse transcriptase activity. A wild-type thermostable DNA polymerase useful for the present invention may or may not possess native reverse transcriptase activity. Useful wild-type thermostable DNA polymerases according to the present invention include, but are not limited to, the polymerases listed in Tables II-IV.

In one embodiment, a wild-type Family B DNA polymerase is used to produce a thermostable DNA polymerase with increased reverse transcriptase activity.

Thermostable archaeal Family B DNA polymerases are typically isolated from Archeobacteria. Archeobacterial organisms from which archaeal Family B DNA polymerases useful in the present invention may be obtained are shown, but not limited to the species shown, in Table IV. The Archaebacteria include a group of “hyperthermophiles” that grow optimally around 100° C. These organisms grow at temperatures higher than 90^{□}C and their enzymes demonstrate greater themostability (Mathur et al., 1992, Stratagies 5:11) than the thermophilic eubacterial DNA polymerases. They are presently represented by three distinct genera, Pyrodictium, Pyrococcus, and Pyrobaculum. Pryodictium brockii (Tₒₚₜ 105° C.) is an obligate autotroph which obtains energy be reducing S^{o} to H₂S with H₂, while Pyrobaculum islandicum (Tₒₚₜ 100° C.) is a faculative heterotroph that uses either organic substrates or H₂ to reduce S^{o}. In contrast, Pyrococcus furiosus (Tₒₚₜ 100° C.) grows by a fermentative-type metabolism rather than by S^{o} respiration. It is a strict heterotroph that utilizes both simple and complex carbohydrates where only H₂ and CO₂ are the detectable products. The organism reduces elemental sulfur to H₂S apparently as a form of detoxification since H₂ inhibits growth.

The starting sequences for the generation of Family B DNA polymerases according to the invention may be contained in a plasmid vector. A non-limiting list of cloned Family B DNA polymerases and their GenBank Accession numbers are listed in Table III

TABLE II
DNA POLYMERASE FAMILIES

Refer-
ence

FAMILY A DNA POLYMERASES

| | |
|---|---|
| Bacterial DNA Polymerases | |
| a) E. coli DNA polymerase I | (1) |
| b) Streptococcus pneumoniae DNA polymerase I | (2) |
| c) Thermus aquaticus DNA polymerase I | (3) |
| d) Thermus flavus DNA polymerase I | (4) |
| e) Thermotoga maritima DNA polymerase I | |
| Bacteriophage DNA Polymerases | |
| a) T5 DNA polymerase | (5) |
| b) T7 DNA polymerase | (6) |
| c) Spo1 DNA polymerase | (7) |
| d) Spo2 DNA polymerase | (8) |
| Mitochondrial DNA polymerase | |
| Yeast Mitochondrial DNA polymerase II | (9, 10, |
| | 11) |

FAMILY B DNA POLYMERASES

| | |
|---|---|
| Bacterial DNA polymerase | |
| E. coli DNA polymerase II | (15) |
| Bacteriophage DNA polymerase | |
| a) PRD1 DNA polymerase | (16, |
| | 17) |
| b) φ29 DNA polymerase | (18) |
| c) M2 DNA polymerase | (19) |
| d) T4 DNA polymerase | (20) |
| Archaeal DNA polymerase | |
| a) Thermococcus litoralis DNA polymerase (Vent) | (21, 87, |
| | 88, 89) |
| b) Pyrococcus sp. DNA polymerase (Deep Vent, from | (90) |
| Pyrococcus sp. GB-D) | |
| c) Pyrococcus furiosus DNA polymerase | (22, 91, |
| | 92, 93, |
| | 94) |
| d) Sulfolobus solfataricus DNA polymerase | (23) |
| e) Thermococcus gorgonarius DNA polymerase | (64) |
| f) Thermococcus species TY | (65) |
| g) Thermococcus species strain KODI (formerly | (66, 95) |
| classified as Pyrococcus) | |
| h) JDF-3 DNA polymerase | (96) |
| i) Sulfolobus acidocaldarius | (67, 97, |
| | 98, 99, |
| | 100, 101, |
| | 102, 103) |
| j) Thermococcus species 9^{o}N-7 | (68) |
| k) Pyrodictium occultum | (69) |
| l) Methanococcus voltae | (70) |
| m) Desulfurococcus strain TOK (D. Tok Pol) | (71) |
| Eukaryotic Cell DNA polymerase | |
| (1) DNA polymerase alpha | |
| a) Human DNA polymerase (alpha) | (24) |
| b) S. cerevisiae DNA polymerase (alpha) | (25) |
| c) S. pombe DNA polymerase I (alpha) | (26) |
| d) Drosophila melanogaster DNA polymerase (alpha) | (27) |
| e) Trypanosoma brucei DNA polymerase (alpha) | (28) |
| (2) DNA polymerase delta | |
| a) Human DNA polymerase (delta) | (29, 30) |
| b) Bovine DNA polymerase (delta) | (31) |
| c) S. cerevisiae DNA polymerase III (delta) | (32) |
| d) S. pombe DNA polymerase III (delta) | (33) |
| e) Plasmodiun falciparum DNA polymerase (delta) | (34) |
| (3) DNA polymerase epsilon | |
| S. cerevisiae DNA polymerase II (epsilon) | (35) |
| (4) Other eukaryotic DNA polymerase | |
| S. cerevisiae DNA polymerase Rev3 | (36) |
| Viral DNA polymerases | |
| a) Herpes Simplex virus type 1 DNA polymerase | (37) |
| b) Equine herpes virus type 1 DNA polymerase | (38) |
| c) Varicella-Zoster virus DNA polymerase | (39) |
| d) Epstein-Barr virus DNA polymerase | (40) |
| e) Herpesvirus saimiri DNA polymerase | (41) |
| f) Human cytomegalovirus DNA polymerase | (42) |
| g) Murine cytomegalovirus DNA polymerase | (43) |
| h) Human herpes virus type 6 DNA polymerase | (44) |
| i) Channel Catfish virus DNA polymerase | (45) |
| j) Chlorella virus DNA polymerase | (46) |
| k) Fowlpox virus DNA polymerase | (47) |
| l) Vaccinia virus DNA polymerase | (48) |
| m) Choristoneura biennis DNA polymerase | (49) |
| n) Autographa California nuclear polymerase virus | |
| (AcMNPV) | |
| DNA polymerase | (50) |
| o) Lymantria dispar nuclear polyhedrosis virus DNA | (51) |
| polymerase | |
| p) Adenovirus-2 DNA polymerase | (52) |
| q) Adenovirus-7 DNA polymerase | (53) |
| r) Adenovirus-12 DNA polymerase | (54) |
| Eukaryotic linear DNA plasmid encoded DNA polymerases | |
| a) S-1 Maize DNA polymerase | (55) |
| b) kalilo neurospora intermedia DNA polymerase | (56) |
| c) pA12 ascobolus immersus DNA polymerase | (57) |
| d) pCLK1 Claviceps purpurea DNA polymerase | (58) |
| e) maranhar neurospora crassa DNA polymerase | (59) |
| f) pEM Agaricus bitorquis DNA polymerase | (60) |
| g) pGKL1 Kluyveromyces lactis DNA polymerase | (61) |
| h) pGKL2 Kluyveromyces lactis DNA polymerase | (62) |
| i) pSKL Saccharomyces kluyveri DNA polymerase | (63) |

TABLE III
ACCESSION INFORMATION FOR CERTAIN
THERMOSTABLE DNA POLYMERASES

Vent Thermococcus litoralis - ACCESSION AAA72101; PID:
g348689; VERSION AAA72101.1 GL: 348689; DBSOURCE locus
THCVDPE accession M74198.1
Thest Thermococcus Sp. (Strain Ty) - ACCESSION O33845;
PID g3913524; VERSION O33845 GI: 3913524; DBSOURCE
swissprot: locus DPOL_THEST, accession O33845
Pab Pyrococcus abyssi - ACCESSION P77916; PID g3913529;
VERSION P77916 GI: 3913529; DBSOURCE swissprot: locus
DPOL_PYRAB, accession P77916
PYRHO Pyrococcus horikoshii - ACCESSION O59610; PID
g3913526; VERSION O59610 GI: 3913526; DBSOURCE
swissprot: locus DPOL_PYRHO, accession O59610
Pyrse Pyrococcus Sp. (Strain Ge23) - ACCESSION P77932;
PID g3913530; VERSION P77932 GI: 3913530; DBSOURCE
swissprot: locus DPOL_PYRSE, accession P77932
Deep Vent Pyrococcus sp. - ACCESSION AAA67131; PID
g436495; VERSION AAA67131.1 GL: 436495; DBSOURCE locus
PSU00707 accession U00707.1
Pfu Pyrococcus furiosus - ACCESSION P80061; PID
g399403; VERSION P80061 GI: 399403; DBSOURCE
swissprot: locus DPOL_PYRFU, accession P80061
JDF-3 -- Thermococcus sp. - ACCESSION AX135459; Baross
gi|2097756|pat|US|5602011|12 Sequence 12 from patent
U.S. Pat. No. 5602011
9^{o}N Thermococcus Sp. (Strain 9^{o}N -7). - ACCESSION
Q56366; PID g3913540; VERSION Q56366 GI: 3913540;
DBSOURCE swissprot: locus DPOL_THES9, accession
Q56366
KOD Pyrococcus sp.- ACCESSION BAA06142; PID g1620911;
VERSION BAA06142.1 GI: 1620911; DBSOURCE locus
PYWKODPOL accession D29671.1
Tgo Thermococcus gorgonarius.- ACCESSION 4699806;
PID g4699806; VERSION GI: 4699806; DBSOURCE pdb:
chain 65, release Feb 23, 1999
THEFM Thermococcus fumicolans; ACCESSION P74918; PID
g3913528; VERSION P74918 GI: 3913528; DBSOURCE
swissprot: locus DPOL_THEFM, accession P74918
METTH Methanobacterium thermoautotrophicum - ACCESSION
O27276; PID g3913522; VERSION O27276 GI: 3913522;
DBSOURCE swissprot: locus DPOL_METTH, accession O27276
Methanococcus jannaschii - ACCESSION Q58295; PID
g3915679; VERSION Q58295 GL: 3915679; DBSOURCE
swissprot: locus DPOL_METJA, accession Q58295
POC Pyrodictium occultum- ACCESSION B56277; PID
g1363344; VERSION B56277 GI: 1363344; DBSOURCE pir:
locus B56277
ApeI Aeropyrum pernix; ACCESSION BAA81109; PID
g5105797; VERSION BAA81109.1 GI: 5105797; DBSOURCE
locus AP000063 accession AP000063.1
ARCFU Archaeoglobus fulgidus - ACCESSION O29753;
PID g3122019; VERSION O29753 GI: 3122019; DBSOURCE
swissprot: locus DPOL_ARCFU, accession O29753
Desulfurococcus sp. Tok. - ACCESSION 6435708; PID
g64357089; VERSION GT: 6435708; DBSOURCE pdb. chain
65, release Jun 2, 1999

TABLE IV
CRENARCHAEOTA (EXTREMELY THERMOPHILIC ARCHAEBACTERIA)

Thermoprotei
Desulfurococcales; Desulfurococcaceae; Aeropyrum (Aeropyrum pernix); Desulfurococcus
(Desulfurococcus amylolyticus, Desulfurococcus mobilis, Desulfurococcus mucosus,
Desulfurococcus saccharovorans, Desulfurococcus sp, Desulfurococcus sp. SEA,
Desulfurococcus sp. SY, Desulfurococcus sp. Tok, Ignicoccus, Ignicoccus islandicus,
Ignicoccus pacificus, Staphylothermus, Staphylothermus hellenicus (Staphylothermus
marinus); Stetteria (Stetteria hydrogenophila); Sulfophobococcus (Sulfophobococcus
zilligii); Thermodiscus (Thermodiscus maritimus); Thermosphaera (Thermosphaera
aggregans); Pyrodictiaceae; Hyperthermus (Hyperthermus butylicus); Pyrodictium
(Pyrodictium abyssi, Pyrodictium brockii, Pyrodictium occultum); Pyrolobus (Pyrolobus
fumarii); unclassified Desulfurococcales; Acidilobus (Acidilobus aceticus); Caldococcus
(Caldococcus noboribetus); Sulfolobales; Sulfolobaceae; Acidianus (Acidianus ambivalens,
Acidianus brierleyi, Acidianus infernus, Acidianus sp. S5, Metallosphaera, Metallosphaera
prunae, Metallosphaera sedula, Metallosphaera sp., Metallosphaera sp. GIB11/00,
Metallosphaer sp. J1); Stygiolobus (Stygiolobus azoricus); Sulfolobus (Sulfolobus
acidocaldarius, Sulfolobus islandicus, Sulfolobus metallicus, Sulfolobus shibatae, Sulfolobus
solfataricus, Sulfolobus thuringiensis, Sulfolobus tokodaii. Sulfolobus yangmingensis,
Sulfolobus sp., Sulfolobus sp. AMP12/99, Sulfolobus sp. CH7/99, Sulfolobus sp. FF5/00,
Sulfolobus sp. MV2/99, Sulfolobus sp. MVSoil3/SC2, Sulfolobus sp. MVSoil6/SC1,
Sulfolobus sp. NGB23/00,. Sulfolobus sp. NGB6/00, Sulfolobus sp. NL8/00, Sulfolobus sp.
NOB8H2, Sulfolobus sp. RC3, Sulfolobus sp. RC6/00, Sulfolobus sp. RCSC1/01,
Sulfurisphaera, Sulfurisphaera ohwakuensis); Thermoproteales; Thermofiliaceae;
Thermofilum; Thermofilum librum (Thermofilum pendens); unclassified Thermofiliaceae
(Thermofiliaceae str. SRI-325, Thermofiliaceae str. SRI-370); Thermoproteaceae; Caldivirga
(Caldivirga maquilingensis); Pyrobaculum (Pyrobaculum aerophilum. Pyrobaculum
arsenaticum, Pyrobaculum islandicum, Pyrobaculum neutrophilum, Pyrobaculum oguniense,
Pyrobaculum organotrophum, Pyrobaculum sp. WIJ3); Thermocladium (Thermocladium
modestius); Thermoproteus (Thermoproteus neutrophilus, Thermoproteus tenax,
Thermoproteus sp. IC-033, Thermoproteus sp. IC-061); Vulcanisaeta (Vulcanisaeta
distributa, Vulcanisaeta souniana)
Euryarchaeota
Archaeoglobi; Archaeoglobales; Archaeoglobaceae; Archaeoglobus (Archaeoglobus
fulgidus, Archaeoglobus lithotrophicus, Archaeoglobus profundus, Archaeoglobus
veneficus); Ferroglobus (Ferroglobus placidus); Halobacteria; Halobacteriales;
Halobacteriaceae; Haloalcalophilium (Haloalcalophilium atacamensis); Haloarcula
(Haloarcula aidinensis, Haloarcula argentinensis, Haloarcula hispanica, Haloarcula japonica);
Haloarcula marismortui (Haloarcula marismortui subsp. marismortui), Haloarcula
mukohataei, Haloarcula sinaiiensis, Haloarcula vallismortis, Haloarcula sp., Haloarcula sp.
ARG-2); Halobacterium (Halobacterium salinarum (Halobacterium salinarum (strain Mex),
Halobacterium salinarum (strain Port), Halobacterium salinarum (strain Shark)),
Halobacterium sp., Halobacterium sp. 9R, Halobacterium sp. arg-4, Halobacterium sp. AUS-
1, Halobacterium sp. AUS-2, Halobacterium sp. GRB, Halobacterium sp. JP-6,
Halobacterium sp. NCIMB 714, Halobacterium sp. NCIMB 718, Halobacterium sp. NCIMB
720, Halobacterium sp. NCIMB 733, Halobacterium sp. NCIMB 734, Halobacterium sp.
NCIMB 741, Halobacterium sp. NCIMB 765, Halobacterium sp. NRC-1, Halobacterium sp.
NRC-817, Halobacterium sp. SG1, Halobaculum, Halobaculum gomorrense); Halococcus
(Halococcus dombrowskii, Halococcus morrhuae, Halococcus saccharolyticus, Halococcus
salifodinae, Halococcus tibetense, Halococcus sp); Haloferax (Haloferax alexandrinus,
Haloferax alicantei, Haloferax denitrificans, Haloferax gibbonsii, Haloferax mediterranei,
Haloferax volcanii, Haloferax sp., Haloferax sp. D1227, Haloferax sp. LWp2.1);
Halogeometricum (Halogeometricum borinquense); Halorhabdus (Halorhabdus utahensis);
Halorubrum (Halorubrum coriense, Halorubrum distributum, Halorubrum lacusprofundi
Halorubrum saccharovorum, Halorubrum sodomense; Halorubrum tebenquichense,
Halorubrum tibetense, Halorubrum trapanicum, Halorubrum vacuolarum, Halorubrum
sp. GSL5.48, Halorubrum sp. SC1.2); Halosimplex (Halosimplex carlsbadense); aloterrigena
(Haloterrigena thermotolerans, Haloterrigena turkmenicus, Natrialba, Natrialba aegyptia;
Natrialba asiatica, Natrialba chahannaoensis, Natrialba hulunbeirensis, Natrialba magadii,
Natrialba sp. ATCC 43988, Natrialba sp. Tunisia HMg-25, Natrialba sp. Tunisia HMg-27);
Natrinema (Natrimema versiforme, Natrinema sp. R-fish); Natronobacterium
(Natronobacterium gregoryi, Natronobacterium innermongoliae, Natronobacterium
nitratireducens, Natronobacterium wudunaoensis); Natronococcus (Natronococcus
amylolyticus, Natronococcus occultus, Natronococcus xinjiangense, Natronococcus sp.);
Natronomonas (Natronomonas pharaonis); Natronorubrum (Natronorubrum bangense,
Natronorubrum tibetense, Natronorubrum sp. Tenzan-10, Natronorubrum sp. Wadi Natrun-
19).
Methanobacteria
Methanobacteriales; Methanobacteriaceae; Methanobacterium (Methanobacterium bryantii,
Methanobacterium congolense, Methanobacterium curvum, Methanobacterium defluvii,
Methanobacterium espanolae, Methanobacterium formicicum, Methanobacterium ivanovii,
Methanobacterium oryzae, Methanobacterium palustre, Methanobacterium subterraneum,
Methanobacterium thermaggregans, Methanobacterium thermoflexum, Methanobacterium
thermophilum, Methanobacterium uliginosum, Methanobacterium sp.); Methanobrevibacter
(Methanobrevibacter arboriphilus, Methanobrevibacter curvatus, Methanobrevibacter
cuticularis, Methanobrevibacter filiformis, Methanobrevibacter oralis, Methanobrevibacter
ruminantium, Methanobrevibacter smithii, methanogenic endosymbiont of Nyctotherus
cordiformis. methanogenic endosymbiont of Nyctotherus ovalis, methanogenic
endosymbiont of Nyctotherus velox, methanogenic symbiont RS104, methanogenic symbiont
RS105, methanogenic symbiont RS208, methanogenic symbiont RS301, methanogenic
symbiont RS404, Methanobrevibacter sp., Methanobrevibacter sp. ATM,
Methanobrevibacter sp. FMB1, Methanobrevibacter sp. FMB2, Methanobrevibacter sp.
FMB3, Methanobrevibacter sp. FMBK1, Methanobrevibacter sp. FMBK2,
Methanobrevibacter sp. FMBK3, Methanobrevibacter sp. FMBK4, Methanobrevibacter sp.
FMBK5, Methanobrevibacter sp. FMBK6, Methanobrevibacter sp. FMBK7,
Methanobrevibacter sp. HW23, Methanobrevibacter sp. LRsD4, Methanobrevibacter sp.
MD101, Methanobrevibacter sp. MD102, Methanobrevibacter sp. MD103,
Methanobrevibacter sp. MD104, Methanobrevibacter sp. MD105, Methanobrevibacter sp.
RsI3, Methanobrevibacter sp. RsW3, Methanobrevibacter sp. XT106, Methanobrevibacter
sp. XT108, Methanobrevibacter sp. XT109); Methanosphaera (Methanosphaera stadtmanae);
Methanothermobacter; Methanothermobacter marburgensis (Methanothermobacter
marburgensis str. Marburg); Methanothermobacter thermautotrophicus
(Methanothermobacter thermautotrophicus str. Winter, Methanothermobacter wolfeii);
Methanothermaceae; Methanothermus (Methanothermus fervidus, Methanothermus
sociabilis); Methanococci; Methanococcales; Methanococcaceae; Methanococcus
(Methanococcus aeolicus, Methanococcus fervens, Methanococcus igneus, Methanococcus
infernus, Methanococcus jannaschii, Methanococcus maripaludis, Methanococcus vannielii,
Methanococcus voltae, Methanococcus vulcanius, Methanococcus sp. P2F9701a);
Methanothermococcus (Methanothermococcus okinawensis, Methanothermococcus
thermolithotrophicus); Methanomicrobiales; Methanocorpusculaceae; Methanocorpusculum
(Methanocorpusculum aggregans, Methanocorpusculum bavaricum, Methanocorpusculum
labreanum, Methanocorpusculum parvum, Methanocorpusculum sinense, Metopus contortus
archaeal symbiont, Metopus palaeformis endosymbiont, Trimyema sp. archaeal symbiont);
Methanomicrobiaceae; Methanocalculus (Methanocalculus halotolerans, Methanocalculus
taiwanense, Methanocalculus sp. K1F9705b Methanocalculus sp. K1F9705c,
Methanocalculus sp. O1F9702c); Methanoculleus (Methanoculleus bourgensis,
Methanoculleus chikugoensis, Methanoculleus marisnigri, Methanoculleus olentangyi,
Methanoculleus palmolei, Methanoculleus thermophilicus, Methanoculleus sp.,
Methanoculleus sp. BA1, Methanoculleus sp. MAB1, Methanoculleus sp. MAB2,
Methanoculleus sp. MAB3); Methanofollis (Methanofollis aquaemaris, Methanofollis
liminatans, Methanofollis tationis); Methanogenium (Methanogenium cariaci,
Methanogenium frigidum, Methanogenium organophilum, Methanogenium sp.);
Methanomicrobium (Methanomicrobium mobile); Methanoplanus (Methanoplanus
endosymbiosus, Methanoplanus limicola, Methanoplanus petrolearius); Methanospirillum
(Methanospirillum hungatei, Methanospirillum sp.); Methanosarcinales; Methanosaetaceae;
Methanosaeta (Methanosaeta concilii. Methanothrix thermophila, Methanosaeta sp.,
Methanosaeta sp. AMPB-Zg); Methanosarcinaceae; Methanimicrococcus
(Methanimicrococcus blatticola); Methanococcoides (Methanococcoides burtonii,
Methanococcoides methylutens, Methanococcoides sp. NaT1); Methanohalobium
(Methanohalobium evestigatum, Methanohalobium sp. strain SD-1); Methanohalophilus
(Methanohalophilus euhalobius, Methanohalophilus halophilus, Methanohalophilus mahii,
Methanohalophilus oregonensis, Methanohalophilus portucalensis, Methanohalophilus
zhilinae, Methanohalophilus sp. strain Cas-1, Methanohalophilus sp. strain HCM6,
Methanohalophilus sp. strain Ref-1, Methanohalophilus sp. strain SF-1); Methanolobus
(Methanolobus bombayensis, Methanolobus taylorii, Methanolobus tindarius, Methanolobus
vulcani; Methanomethylovorans (Methanomethylovorans hollandica, Methanomethylovorans
victoriae); Methanosarcina (Methanosarcina acetivorans, Methanosarcina barkeri,
Methanosarcina lacustris, Methanosarcina mazei, Methanosarcina semesiae, Methanosarcina
siciliae, Methanosarcina thermophila, Methanosarcina vacuolata, Methanosarcina sp.,
Methanosarcina sp. FR, Methanosarcina sp. GS1-A, Methanosarcina sp. WH-1);
Methanopyri; Methanopyrales; Methanopyraceae; Methanopyrus (Methanopyrus kandleri);
Thermococci; Thermococcales; Thermococcaceae; Palaeococcus (Palaeococcus ferrophilus);
Pyrococcus (Pyrococcus abyssi, Pyrococcus endeavori, Pyrococcus furiosus, Pyrococcus
furiosus DSM 3638, Pyrococcus glycovorans, Pyrococcus horikoshii, Pyrococcus woesei,
Pyrococcus sp., Pyrococcus sp. GB-3A, Pyrococcus sp. GB-D, Pyrococcus sp. GE23,
Pyrococcus sp. GI-H, Pyrococcus sp. GI-J, Pyrococcus sp. JT1, Pyrococcus sp. MZ14,
Pyrococcus sp. MZ4, Pyrococcus sp. ST700); Thermococcus (Thermococcus
acidaminovorans, Thermococcus aegaeus, Thermococcus aggregans, Thermococcus
alcaliphilus, Thermococcus atlantis, Thermococcus barophilus, Thermococcus barossii,
Thermococcus celer, Thermococcus chitonophagus, Thermococcus fumicolans,
Thermococcus gammatolerans, Thermococcus gorgonarius, Thermococcus guaymasensis,
Thermococcus hydrothermalis, Thermococcus kodakaraensis, Thermococcus litoralis,
Thermococcus marinus, Thermococcus mexicalis, Thermococcus pacificus, Thermococcus
peptonophilus, Thermococcus profundus, Thermococcus radiophilus, Thermococcus
sibiricus, Thermococcus siculi, Thermococcus stetteri, Thermococcus sulfurophilus,
Thermococcus waimanguensis, Thermococcus waiotapuensis, Thermococcus zilligii,
Thermococcus sp., Thermococcus sp. 9N2, Thermococcus sp. 9N3, Thermococcus sp. 9oN-
7, Thermococcus sp. B1001, Thermococcus sp. CAR-80, Thermococcus sp. CKU-1,
Thermococcus sp. CKU-199, Thermococcus sp. CL1, Thermococcus sp. CL2, Thermococcus
sp. CMI, Thermococcus sp. CNR-5, Thermococcus sp. CX1, Thermococcus sp. CX2,
Thermococcus sp. CX3, Thermococcus sp. CX4, Thermococcus sp. CYA, Thermococcus sp.
GE8, Thermococcus sp. Gorda2, Thermococcus sp. Gorda3, Thermococcus sp. Gorda4,
Thermococcus sp. Gorda5, Thermococcus sp. Gorda6, Thermococcus sp. JDF-3,
Thermococcus sp. KS-1, Thermococcus sp. KS-8, Thermococcus sp. MZ1, Thermococcus
sp. MZ10, Thermococcus sp. MZ11, Thermococcus sp. MZ12, Thermococcus sp. MZ13,
Thermococcus sp. MZ2, Thermococcus sp. MZ3, Thermococcus sp. MZ5, Thermococcus sp.
MZ6, Thermococcus sp. MZ8, Thermococcus sp. MZ9, Thermococcus sp. P6,
Thermococcus sp. Rt3, Thermococcus sp. SN531, Thermococcus sp. TK1, Thermococcus sp.
vp197); Thermoplasmata; Thermoplasmatales; Ferroplasmaceae; Ferroplasma (Ferroplasma
acidarmanus, Ferroplasma acidiphilum, Picrophilaceae); Picrophilus (Picrophilus oshimae,
Picrophilus torridus; Thermoplasmataceae; Thermoplasma (Thermoplasma acidophilum,
Thermoplasma volcanium, Thermoplasma sp. XT101, Thermoplasma sp. XT102,
Thermoplasma sp. XT103, Thermoplasma sp. XT107);

Korarchaeota (korarchaeote SRI-306).
Preparing Mutant Thermostable DNA Polymerase with Increased Reverse Transcriptase (RT) Activity.

Cloned wild type or mutant DNA polymerases may be modified to generate mutant forms exhibiting increased RT activity by a number of methods. These include the methods described below and other methods known in the art. Any thermostable DNA polymerase can be used to prepare the DNA polymerase mutants with increased RT activity in the invention.

A preferred method of preparing a DNA polymerase with increased RT activity is by genetic modification (e.g., by modifying the DNA sequence encoding a wild type or mutant DNA polymerase). A number of methods are known in the art that permit the random as well as targeted mutation of DNA sequences (see for example, Ausubel et. al. Short Protocols in Molecular Biology (1995)₃rd Ed. John Wiley & Sons, Inc.).

First, methods of random mutagenesis, which will result in a panel of mutants bearing one or more randomly situated mutations, exist in the art. Such a panel of mutants may then be screened for those exhibiting increased RT activity relative to a wild-type polymerase (see “Methods of Evaluating Mutants for Increased RT Activity”, below). An example of a method for random mutagenesis is the so-called “error-prone PCR method”. As the name implies, the method amplifies a given sequence under conditions in which the DNA polymerase does not support high fidelity incorporation. The conditions encouraging error-prone incorporation for different DNA polymerases vary, however one skilled in the art may determine such conditions for a given enzyme. A key variable for many DNA polymerases in the fidelity of amplification is, for example, the type and concentration of divalent metal ion in the buffer. The use of manganese ion and/or variation of the magnesium or manganese ion concentration may therefore be applied to influence the error rate of the polymerase.

Second, there are a number of site-directed mutagenesis methods known in the art, which allow one to mutate a particular site or region in a straightforward manner. There are a number of kits available commercially for the performance of site-directed mutagenesis, including both conventional and PCR-based methods. Useful examples include the EXSITE™ PCR-Based Site-directed Mutagenesis Kit available from Stratagene (Catalog No. 200502; PCR based) and the QUIKCHANGE™ Site-directed mutagenesis Kit from Stratagene (Catalog No. 200518; non-PCR-based), and the CHAMELEON® double-stranded Site-directed mutagenesis kit, also from Stratagene (Catalog No. 200509).

In addition DNA polymerases with increased RT activity may be generated by insertional mutation or truncation (N-terminal, internal or C-terminal) according to methodology known to a person skilled in the art.

Older methods of site-directed mutagenesis known in the art relied upon sub-cloning of the sequence to be mutated into a vector, such as an M13 bacteriophage vector, that allows the isolation of single-stranded DNA template. In these methods one annealed a mutagenic primer (i.e., a primer capable of annealing to the site to be mutated but bearing one or mismatched nucleotides at the site to be mutated) to the single-stranded template and then polymerized the complement of the template starting from the 3′ end of the mutagenic primer. The resulting duplexes were then transformed into host bacteria and plaques were screened for the desired mutation.

More recently, site-directed mutagenesis has employed PCR methodologies, which have the advantage of not requiring a single-stranded template. In addition, methods have been developed that do not require sub-cloning. Several issues may be considered when PCR-based site-directed mutagenesis is performed. First, in these methods it may be desirable to reduce the number of PCR cycles to prevent expansion of undesired mutations introduced by the polymerase. Second, a selection may be employed in order to reduce the number of non-mutated parental molecules persisting in the reaction. Third, an extended-length PCR method may be preferred in order to allow the use of a single PCR primer set. And fourth, because of the non-template-dependent terminal extension activity of some thermostable polymerases it may be necessary to incorporate an end-polishing step into the procedure prior to blunt-end ligation of the PCR-generated mutant product.

In some embodiments, a wild-type DNA polymerase is cloned by isolating genomic DNA or cDNA using molecular biological methods to serve as a template for mutagenesis. Alternatively, the genomic DNA or cDNA may be amplified by PCR and the PCR product may be used as template for mutagenesis.

The unlimiting protocol described below accommodates these considerations through the following steps. First, the template concentration used is approximately 1000-fold higher than that used in conventional PCR reactions, allowing a reduction in the number of cycles from 25-30 down to 5-10 without dramatically reducing product yield. Second, the restriction endonuclease DpnI (recognition target sequence: 5-Gm6ATC-3, where the A residue is methylated) is used to select against parental DNA, since most common strains of E. coli Dam methylate their DNA at the sequence 5-GATC-3 (SEQ ID NO:24). Third, Taq Extender is used in the PCR mix in order to increase the proportion of long (i.e., full plasmid length) PCR products. Finally, Pfu DNA polymerase is used to polish the ends of the PCR product prior to intramolecular ligation using T4 DNA ligase.

One method is described in detail as follows for PCR-based site directed mutagenesis according to one embodiment of the invention.

Plasmid template DNA comprising a DNA polymerase encoding polynucleotide (approximately 0.5 pmole) is added to a PCR cocktail containing: 1× mutagenesis buffer (20 mM Tris HCl, pH 7.5; 8 mM MgCl₂; 40 μg/ml BSA); 12-20 pmole of each primer (one of skill in the art may design a mutagenic primer as necessary, giving consideration to those factors such as base composition, primer length and intended buffer salt concentrations that affect the annealing characteristics of oligonucleotide primers; one primer must contain the desired mutation within the DNA polymerase encoding sequence, and one (the same or the other) must contain a 5′ phosphate to facilitate later ligation), 250 uM each dNTP, 2.5 U Taq DNA polymerase, and 2.5 U of Taq Extender (Available from Stratagene; See Nielson et al. (1994) Strategies 7: 27, and U.S. Pat. No. 5,556,772).

Primers can be prepared using the triester method of Matteucci et al., 1981, J. Am. Chem. Soc. 103:3185-3191, incorporated herein by reference. Alternatively automated synthesis may be preferred, for example, on a Biosearch 8700 DNA Synthesizer using cyanoethyl phosphoramidite chemistry.

The PCR cycling is performed as follows: 1 cycle of 4 min at 94° C., 2 min at 50° C. and 2 min at 72° C.; followed by 5-10 cycles of 1 min at 94° C., 2 min at 54° C. and 1 min at 72° C. The parental template DNA and the linear, PCR-generated DNA incorporating the mutagenic primer are treated with DpnI (10 U) and Pfu DNA polymerase (2.5U). This results in the DpnI digestion of the in vivo methylated parental template and hybrid DNA and the removal, by Pfu DNA polymerase, of the non-template-directed Taq DNA polymerase-extended base(s) on the linear PCR product. The reaction is incubated at 37° C. for 30 min and then transferred to 72° C. for an additional 30 min. Mutagenesis buffer (115 μl of 1×) containing 0.5 mM ATP is added to the DpnI-digested, Pfu DNA polymerase-polished PCR products. The solution is mixed and 10 μl are removed to a new microfuge tube and T4 DNA ligase (2-4 U) is added. The ligation is incubated for greater than 60 min at 37° C. Finally, the treated solution is transformed into competent E. coli according to standard methods.

Direct comparison of Family B DNA polymerases from diverse organisms, including thermostable Family B DNA polymerases indicates that the domain structure of these enzymes is highly conserved (See, e.g., Hopfner et al., 1999, Proc. Natl. Acad. Sci. U.S.A. 96: 3600-3605; Blanco et al., 1991, Gene 100: 27-38; and Larder et al., 1987, EMBO J. 6: 169-175). All Family B DNA polymerases have six conserved regions, designated Regions I-VI, and arranged in the polypeptides in the order IV-II-VI-III-I-V (separation between the Regions varies, but the order does not). Region I (also known as Motif C) is defined by the conserved sequence D T D, located at amino acids 541-543 in Pfu DNA polymerase and at amino acids 540-542 in JDF-3 DNA polymerase. Region II (also known as Motif A) is defined by the consensus sequence D X X (A/S) L Y P S I (SEQ ID NO:25), locatred at amino acids 405-413 in Pfu DNA polymerase and at amino acids 404-412 in JDF-3 DNA polymerase. Region III (also known as Motif B) is defined by the consensus sequence K X X X N A/S X Y G (SEQ ID NO:26), located at amino acids 488-496 in Pfu DNA polymerase and at amino acids 487-495 in JDF-3 DNA polymerase. Sequence alignments of these sequences with those of other Family B DNA polymerases permit the assignment of the boundaries of the various Regions on other Family B DNA polymerases. The crystal structures have been solved for several Family B DNA polymerases, including Thermococcus gorgonarius (Hopfner et al., 1999, Proc. Natl. Acad. Sci. U.S.A. 96: 3600-3605), 9^{o}N (Rodrigues et al., 2000, J. Mol. Biol. 299: 447-462), and Thermococcus sp. strain KODI (formerly classified as a Pyrococcus sp., Hashimoto et al., 2001, J. Mol. Biol. 306: 469-477), aiding in the establishment of structure/function relationships for the Regions. The location of these conserved regions provides a useful model to direct genetic modifications for preparing DNA polymerase with increased RT activity whilst conserving essential functions e.g. DNA polymerization and proofreading activity. For example, it is recognized herein that the “LYP” structural motif that is part of the larger conserved structural motif DXXSLYPSI (SEQ ID NO:27) defining Region II is a primary target for mutations that enhance the reverse transcriptase activity of the enzyme. As used herein, the term “LYP motif” means an amino acid sequence within Region II of a Family B DNA polymerase that corresponds in a sequence alignment, performed using BLAST or Clustal W, to the LYP sequence located at amino acids 408 to 410 of the JDF-3 Family B DNA polymerase of SEQ ID NO: 1 (the LYP motif of Pfu DNA polymerase is located at amino acids 409-411 of the polypeptide). It is noted that while the motif is most frequently LYP, there are members of the Family B DNA polymerases that vary in this motif—for example, the LYP corresponds to MYP in Archaeoglobus fulgidusfu (Afu) DNA polymerase.

As disclosed herein, amino acid changes at the position corresponding to L408 of SEQ ID NO: 1 which lead to increased reverse transcriptase activity tend to introduce cyclic side chains, such as phenylalanine, tryptophan, histidine or tyrosine. While the amino acids with cyclic side chains are demonstrated herein to increase the reverse transcriptase activity of Family B DNA polymerases, other amino acid changes at the LYP motif are contemplated to have effects on the reverse transcriptase activity. Thus, in order to modify the reverse transcriptase activity of another Family B DNA polymerase, one would first look to modify the LYP motif of Region II, particularly the L or other corresponding amino acid of the LYP motif, first substituting cyclic side chains and assessing reverse transcriptase activity relative to wild-type as disclosed herein below in “Methods of Evaluating Mutants for Increased RT Activity”. If necessary or if desired, one can subsequently modify the same position in the LYP motif with additional amino acids and similarly assess the effect on activity. Alternatively, or in addition, one can modify the other positions in the LYP motif and similarly assess the reverse transcriptase activity.

A degenerate oligonucleotide primer may be used for generating DNA polymerase mutants of the present invention. In some embodiments, chemical synthesis of a degenerate primer is carried out in an automatic DNA synthesizer, and the purpose of a degenerate primer is to provide, in one mixture, all of the sequences encoding a specific desired mutation site of the DNA polymerase sequence. The synthesis of degenerate oligonucleotides is well known in the art (e.g., Narang, S. A, Tetrahedron 39:3 9, 1983; Itakura et al., Recombinant DNA, Proc 3rd Cleveland Sympos. Macromol., Walton, ed., Elsevier, Amsterdam, pp 273-289, 1981; Itakura et al., Annu. Rev. Biochem. 53:323, 1984; Itakura et al., Science 198:1056, 1984; and Ike et al., Nucleic Acid Res. 11:477 1983). Such techniques have been employed in the directed evolution of other proteins (e.g., Scott et al., Science 249:386-390, 1980; Roberts et al., Proc. Nat'l. Acad. Sci., 89:2429-2433, 1992; Devlin et al., Science 249: 404-406, 1990; Cwirla et al., Proc. Nat'l. Acad. Sci., 87: 6378-6382, 1990; as well as U.S. Pat. Nos. 5,223,409, 5,198,346, and 5,096,815, each of which is incorporated herein by reference).

A polynucleotide encoding a mutant DNA polymerase with increased RT activity may be screened and/or confirmed by methods known in the art, such as described below in Methods of Evaluating Mutants for Increased RT Activity.

Polynucleotides encoding the desired mutant DNA polymerases generated by mutagenesis may be sequenced to identify the mutations. For those mutants comprising more than one mutation, the effect of a given mutation may be evaluated by introduction of the identified mutation to the wild-type gene by site-directed mutagenesis in isolation from the other mutations borne by the particular mutant. Screening assays of the single mutant thus produced will then allow the determination of the effect of that mutation alone.

In a preferred embodiment, the enzyme with increased RT activity is derived from an Family B DNA polymerase containing one or more mutations.

In a preferred embodiment, the enzyme with increased RT activity is derived from a Pfu or JDF-3 DNA polymerase.

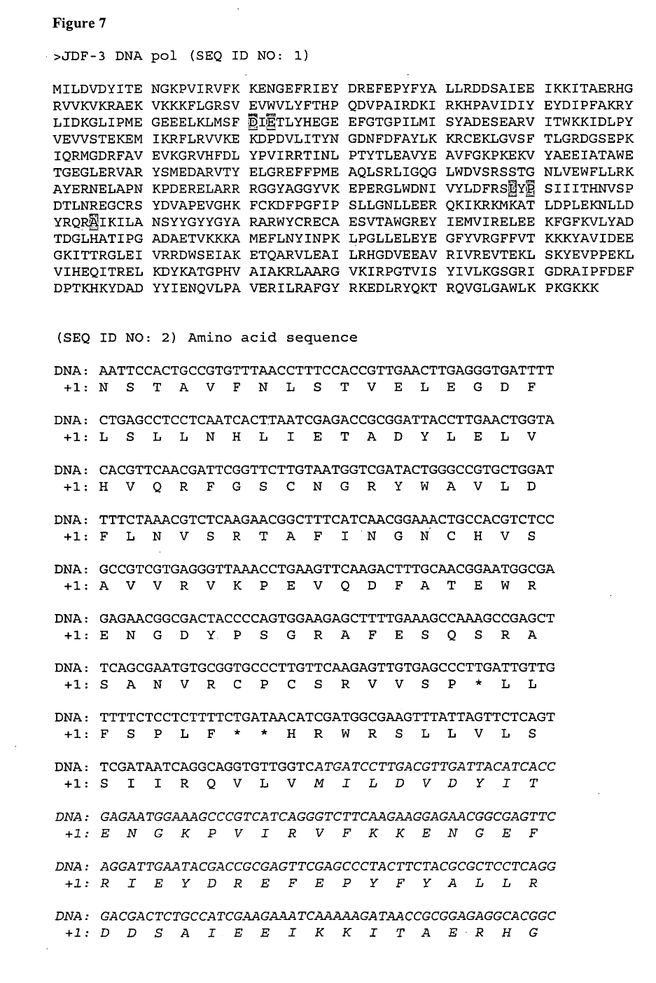

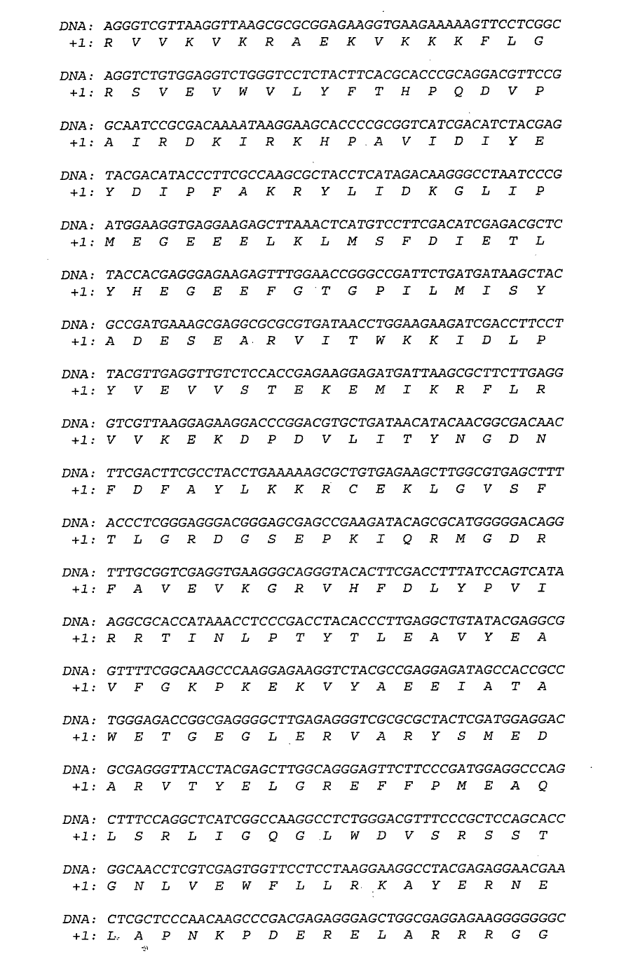

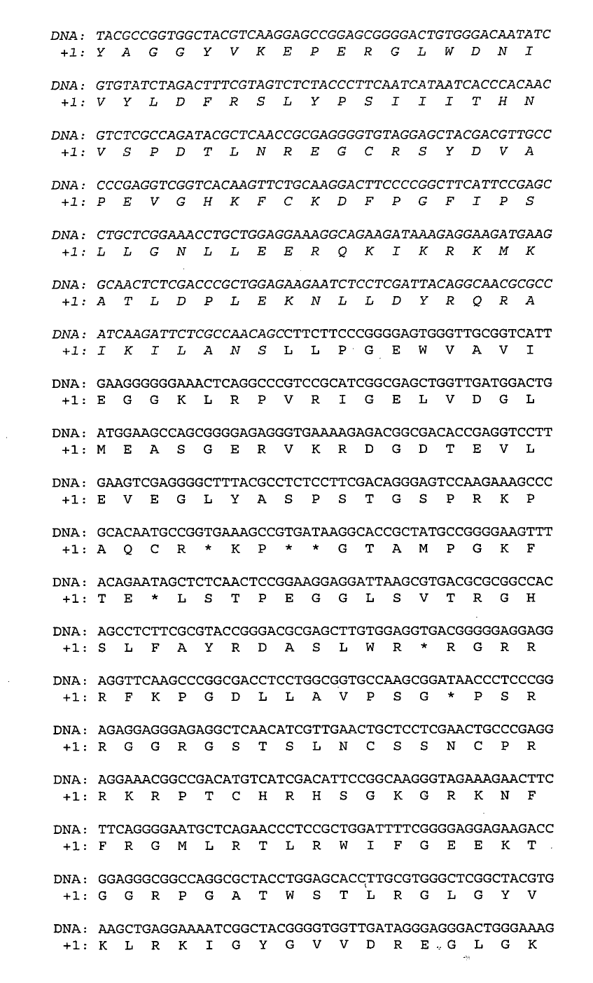

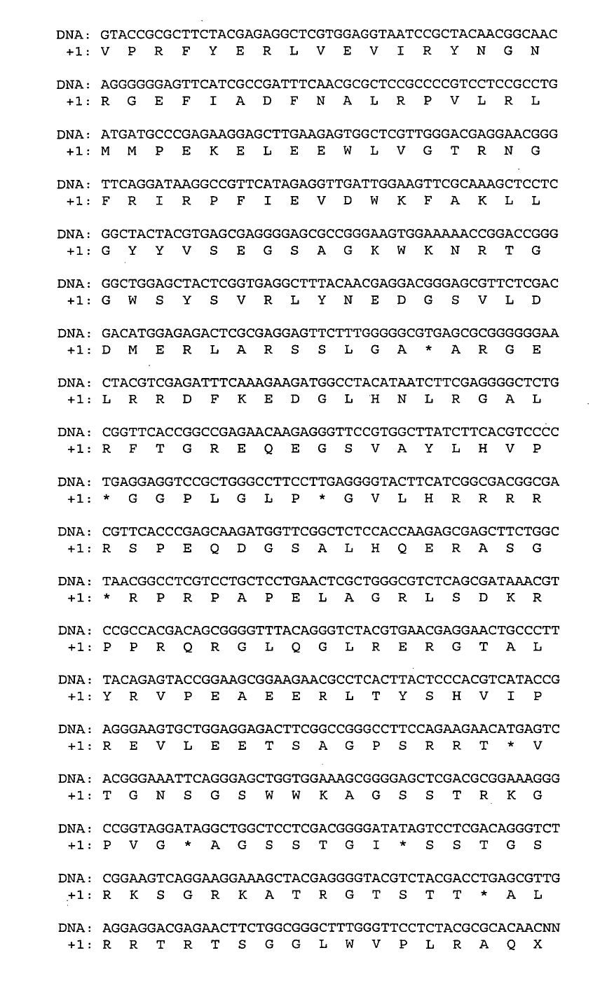

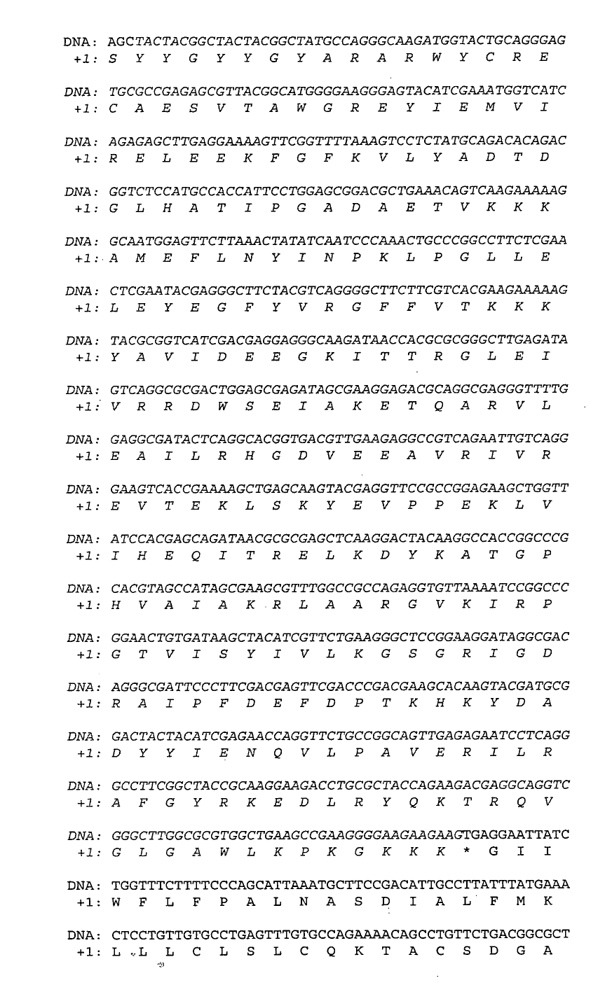

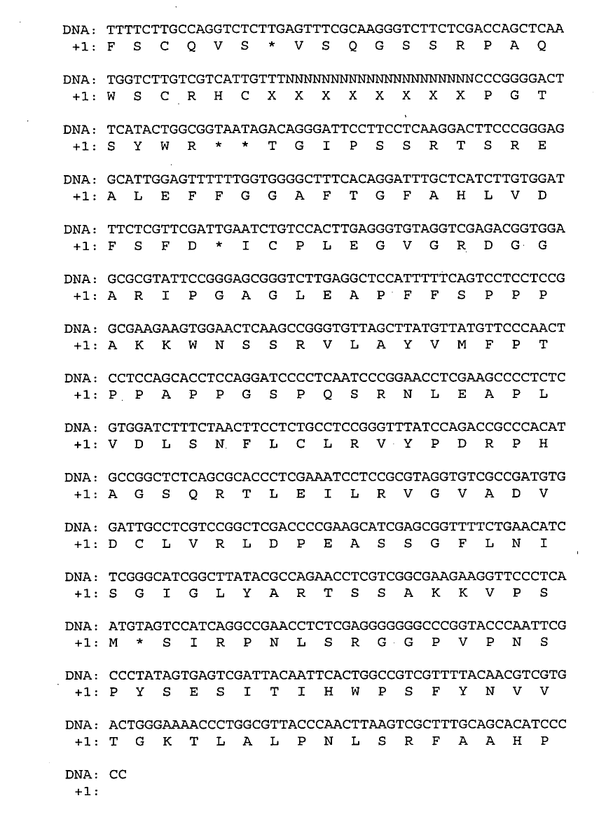

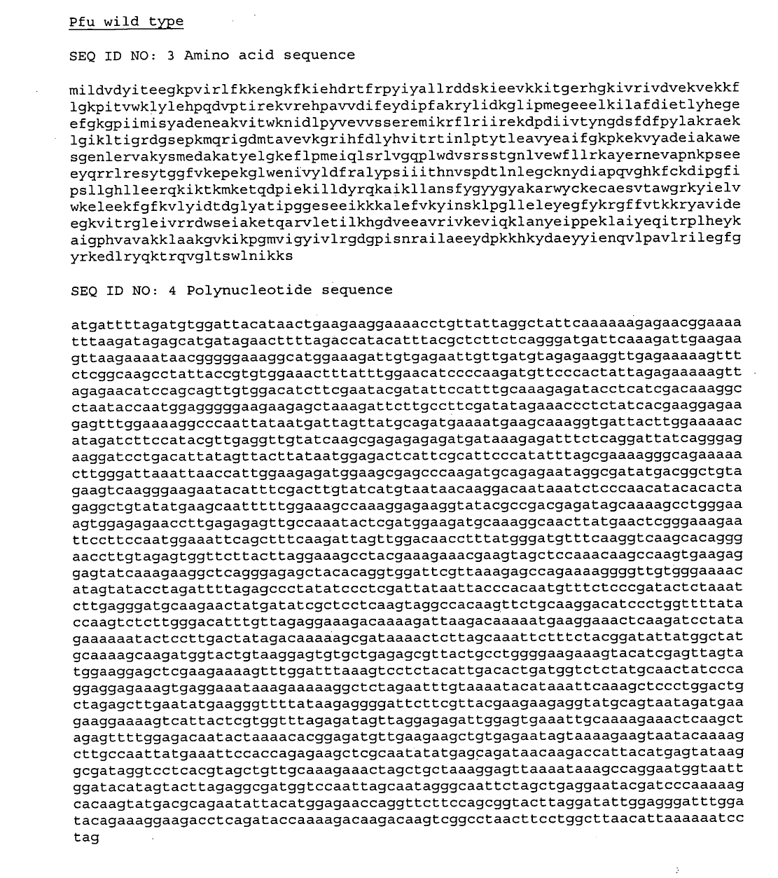

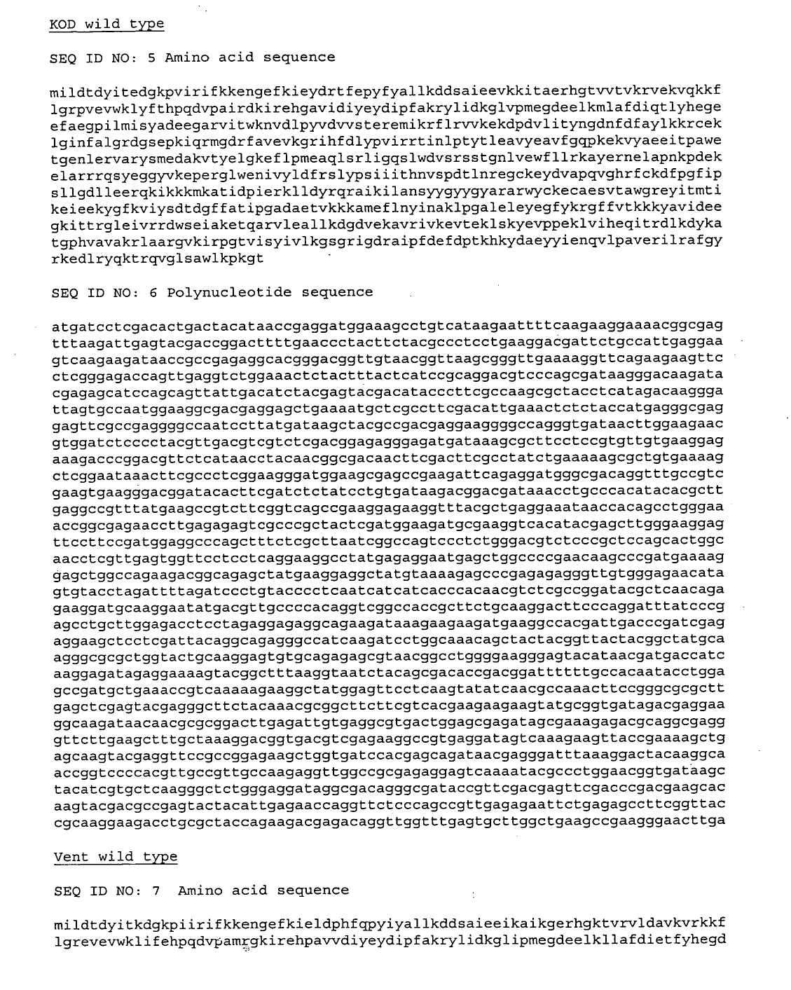

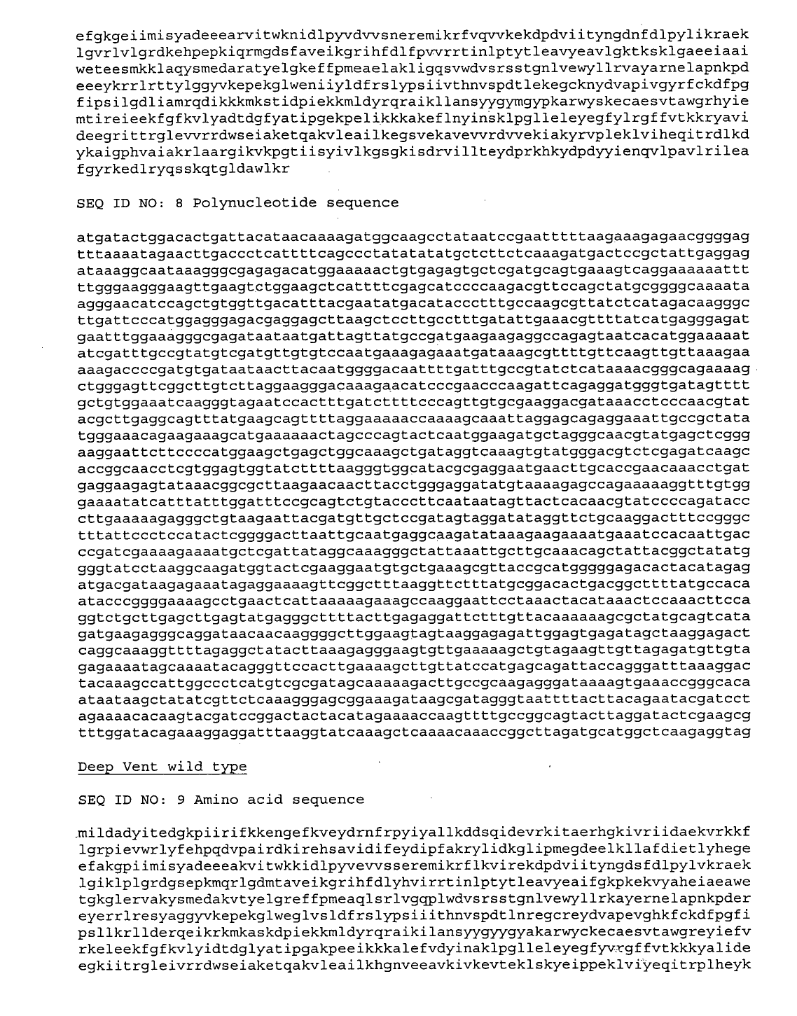

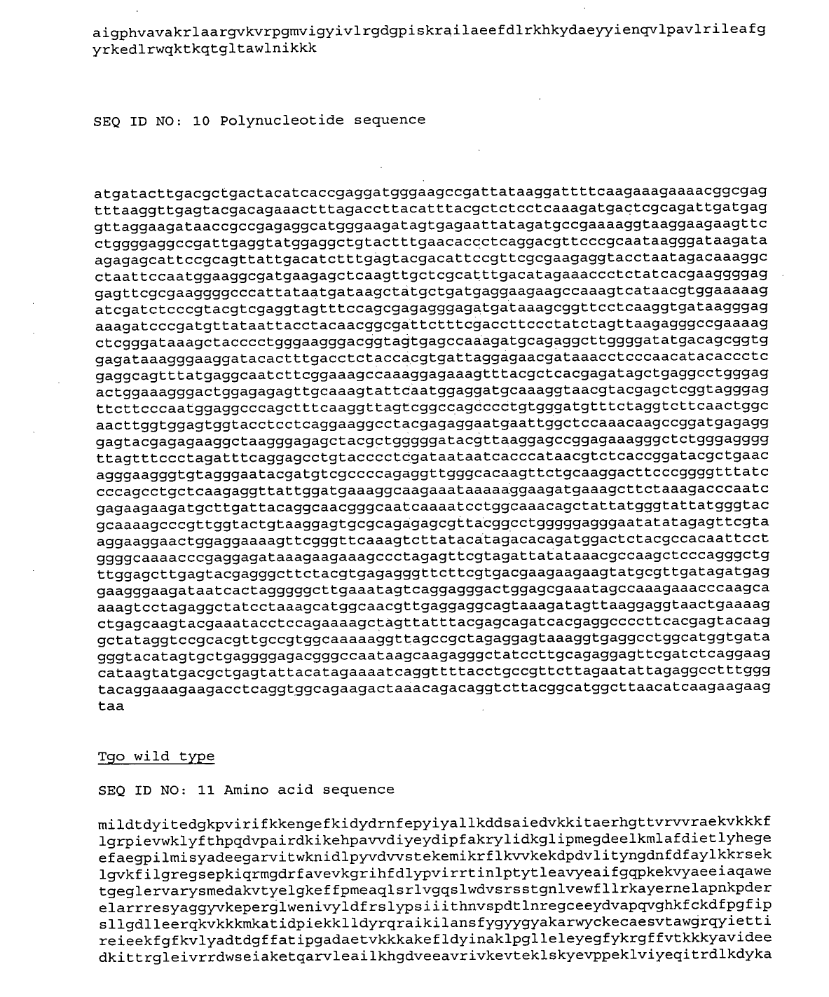

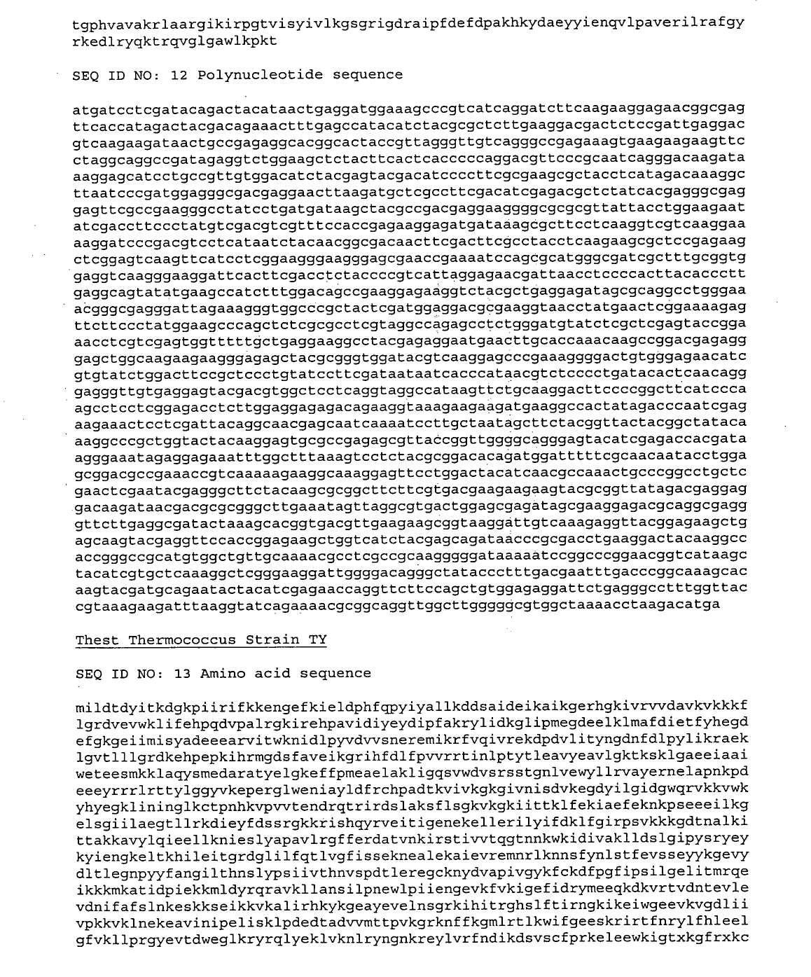

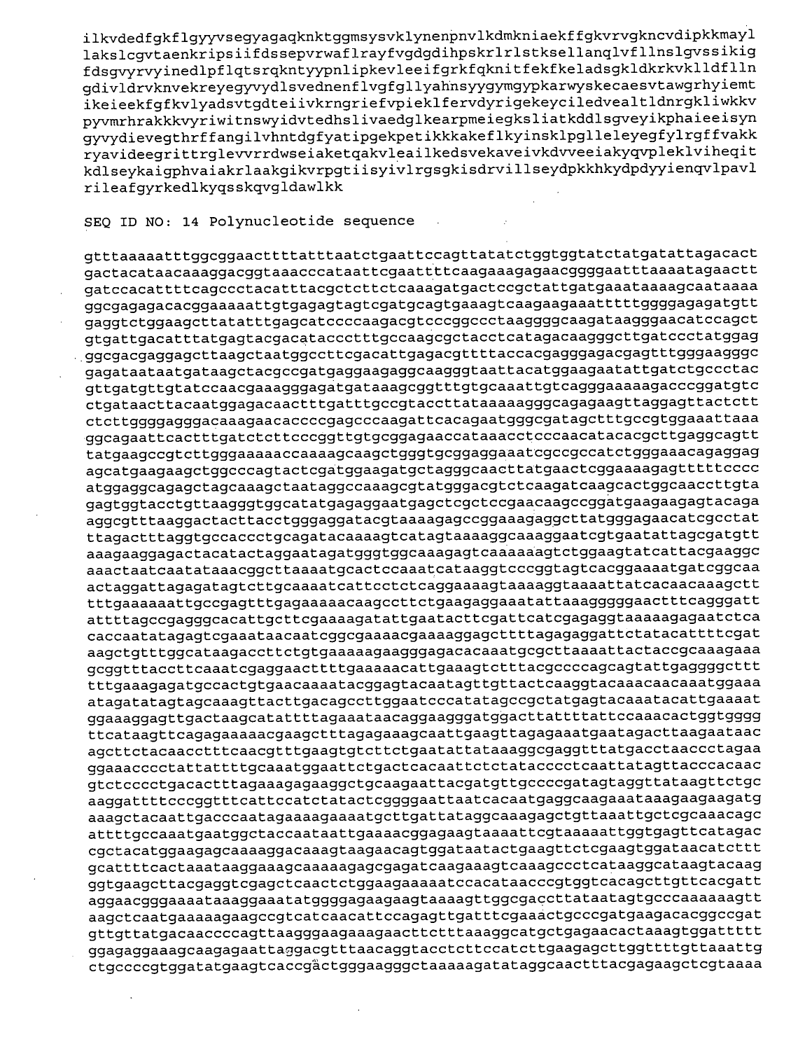

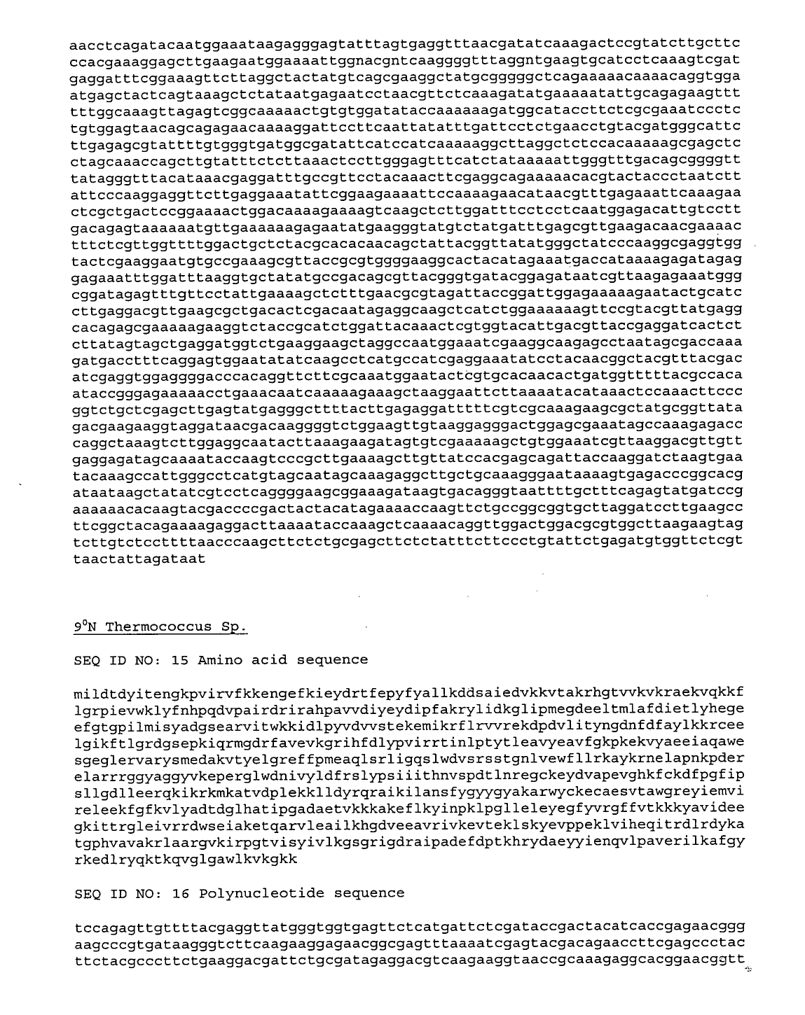

The amino acid and DNA coding sequence of a wild-type Pfu or JDF-3 DNA polymerase are shown in FIG. 7 (Genbank Accession # P80061 (PFU) and Q56366 (JDF-3), respectively). A detailed description of the structure and function of Pfu DNA polymerase can be found, among other places, in U.S. Pat. Nos. 5,948,663; 5,866,395; 5,545,552; 5,556,772, while a detailed description of the structure and function of JDF-3 DNA polymerase can be found, among other places, in U.S. Pat. Nos. 5,948,663; 5,866,395; 5,545,552; 5,556,772, all of which are hereby incorporated by reference. A non-limiting detailed procedure for preparing Pfu or a JDF-3 DNA polymerase with increased RT activity is provided in the Examples herein.

A person of ordinary skill in the art having the benefit of this disclosure will recognize that polymerases with reduced uracil detection activity derived from Family B DNA polymerases, including Vent DNA polymerase, JDF-3 DNA polymerase, Pfu polymerase, Tgo DNA polymerase, KOD, other enzymes listed in Tables II and III, and the like may be suitably used in the present invention.

The enzyme of the subject composition may comprise DNA polymerases that have not yet been isolated.

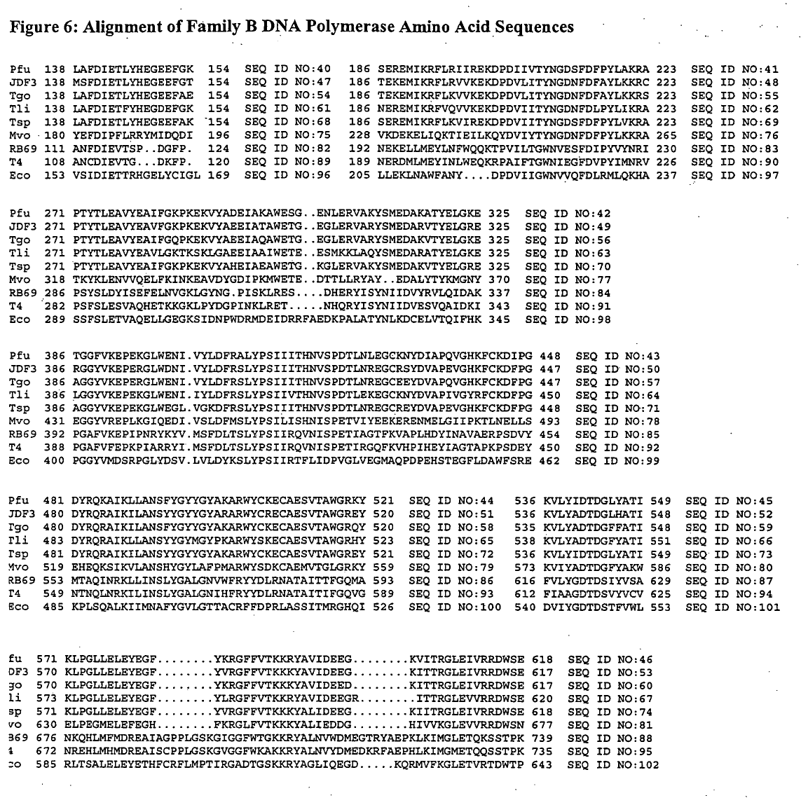

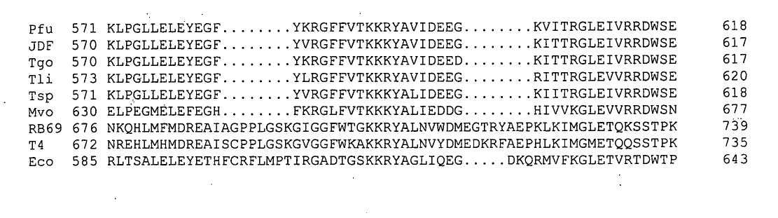

In preferred embodiments of the invention, the mutant Family B DNA polymerase harbours an amino acid substitution at amino acid position corresponding to L409 of the Pfu DNA polymerase (see FIG. 6). In a preferred embodiment, the mutant DNA polymerase of the invention contains a Leucine to F, Y, W or H substitution at the amino acid at a position corresponding to L408 of the JDF-3 Polymerase or L409 of the Pfu DNA polymerase.

In one embodiment, the mutant DNA polymerase of the present invention is a Pfu DNA polymerase that contains a Leucine to F, Y, W or H substitution at amino acid position 409.

In one embodiment, the mutant DNA polymerase of the present invention is a JDF-3 DNA polymerase that contains a Leucine to F, Y, W or H substitution at amino acid position 408.

In one embodiment, the mutant DNA polymerase contains an amino acid mutation at the amino acids corresponding to L409 to P411 of SEQ ID NO:3.

According to the invention, LYP motif mutant DNA polymerases (e.g., Pfu L409 mutant or JDF-3 L408 mutant) with increased RT activity may contain one or more additional mutations that further increases its RT activity, or reduce or abolish one or more additional activities of the DNA polymerases, e.g., 3′-5′ exonuclease activity, base analog detection activity.

In one embodiment, an L409 mutant Pfu DNA polymerase according to the invention contains one or more additional mutations that result in a form which is substantially lacking 3′-5′ exonuclease activity.

The invention further provides for L409 mutant Pfu DNA polymerases with increased RT activity further containing one or mutations that reduce or eliminate 3′-5′ exonuclease activity as disclosed in the pending U.S. patent application Ser. No. 09/698,341 (Sorge et al; filed Oct. 27, 2000).

In a preferred embodiment, the invention provides for a L409/D141/E143 triple mutant Pfu DNA polymerase with reduced 3′-5′ exonuclease activity and increased RT activity.

In one embodiment, the triple mutant Pfu DNA polymerase contains an F, Y, W or H substitution at L409, an A substitution at D141, and an A substitution at E143.

According to the invention, LYP motif mutant DNA polymerases (e.g., Pfu L409 mutant or JDF-3 L408 mutant) with increased RT activity may contain one or more additional mutations that reduce base analog detection activity.

In one embodiment, an L409 mutant Pfu DNA polymerase according to the invention contains one or more additional mutations that result in a form which exhibits reduced base analog detection activity.

The invention provides for L409 mutant Pfu DNA polymerases with increased RT activity further containing one or mutations that reduce base analog detection activity as disclosed in the pending U.S. patent application Ser. No. 10/408,601 (Hogrefe et al; filed Apr. 7, 2003).

In one embodiment, the invention provides for a L409N93 mutant Pfu DNA polymerase with increased RT activity and reduced base analog detection activity. In another embodiment the mutant Pfu DNA polymerase contains an F, Y, W or H substitution at L409, an A substitution at D141, and a R, E, K, D or B substitution at V93. In another embodiment the mutant Pfu DNA polymerase with increased RT activity and reduced base analog detection activity comprises the amino acid sequence of SEQ ID NO: 105.

In a preferred embodiment, the invention provides for a L409/D141/E143/V93 quadruple mutant Pfu DNA polymerase with reduced 3′-5′ exonuclease activity reduced base analog detection activity and increased RT activity.

In one embodiment, the quadruple mutant Pfu DNA polymerase contains an F, Y, W or H substitution at L409, an A substitution at D141, an A substitution at E143, and a R, E, K, D or B substitution at V93. In another embodiment the quadruple mutant Pfu DNA polymerase comprises the amino acid sequence of SEQ ID NO: 106.

DNA polymerases containing multiple mutations may be generated by site directed mutagenesis using a polynucleotide encoding a DNA polymerase mutant already possessing a desired mutation, or they may be generated by using one or more mutagenic primers containing one or more according to methods that are well known in the art and are described herein.

Methods used to generate 3′-5′ exonuclease deficient JDF-3 DNA polymerases including the D141A and E143A mutations are disclosed in the pending U.S. patent application Ser. No. 09/698,341 (Sorge et al; filed Oct. 27, 2000). A person skilled in the art in possession of the L409 Pfu DNA polymerase cDNA and the teachings of the pending U.S. patent application Ser. No. 09/698,341 (Sorge et al; filed Oct. 27, 2000) would have no difficulty introducing both the corresponding D141A and E143A mutations or other 3′-5′ exonuclease mutations into the L409 Pfu DNA polymerase cDNA, as disclosed in the pending U.S. patent application Ser. No. 09/698,341, using established site directed mutagenesis methodology.

Methods used to generate mutant archaeal DNA polymerases with reduced base analog detection activity including the V93R, V93E, V93K, V93D and V93B mutations are disclosed in the pending U.S. patent application Ser. No. 10/408,601 (Hogrefe et al; filed Apr. 7, 2003). A person skilled in the art in possession of the L409 Pfu DNA polymerase cDNA and the teachings of the pending U.S. patent application Ser. No. 10/408,601 (Hogrefe et al.; filed Apr. 7, 2003) would have no difficulty introducing the V93 mutations or other mutations resulting in reduced based analog detection activity into the L409 Pfu DNA polymerase cDNA, as disclosed in the pending U.S. patent application Ser. No. 10/408,601, using established site directed mutagenesis methodology.

In another embodiment, a mutant Family B DNA polymerase is a chimeric protein, for example, further comprising a domain that increases processivity and/or increases salt resistance. A domain useful according to the invention and methods of preparing chimeras are described in WO 01/92501 A1 and Pavlov et al., 2002, Proc. Natl. Acad. Sci USA, 99:13510-13515. Both references are herein incorporated in their entirety.

In light of the present disclosure, other forms of mutagenesis generally applicable will be apparent to those skilled in the art in addition to the aforementioned mutagenesis methods. For example, DNA polymerase mutants can be generated and screened using, for example, alanine scanning mutagenesis and the like (Ruf et al., Biochem., 33:1565-1572, 1994; Wang et al., J. Biol. Chem., 269:3095-3099, 1994; Balint et al. Gene 137:109-118, 1993; Grodberg et al., Eur. J. Biochem., 218:597-601, 1993; Nagashima et al., J. Biol. Chem., 268:2888-2892, 1993; Lowman et al., Biochem., 30:10832-10838, 1991; and Cunningham et al., Science, 244:1081-1085, 1989); linker scanning mutagenesis (Gustin et al., Virol., 193:653-660, 1993; Brown et al., Mol. Cell. Biol., 12:2644-2652, 1992; McKnight et al., Science, 232:316); or saturation mutagenesis (Meyers et al., Science, 232:613, 1986), all references hereby incorporated by reference.

Methods of Evaluating Mutants for Increased RT Activity.

A wide range of techniques are known in the art for screening polynucleotide products of mutagenesis. The most widely used techniques for screening large number of polynucleotide products typically comprise cloning the mutagenesis polynucleotides into replicable expression vectors, transforming appropriate cells with the resulting vectors, and expressing the polynucleotides under conditions such that detection of a desired activity (e.g., RT) facilitates relatively easy isolation of the vector containing the polynucleotide encoding the desired product.

Methods for assaying reverse transcriptase (RT) activity based on the RNA-dependent synthesis of DNA have been well known in the art, e.g., as described in U.S. Pat. No. 3,755,086; Poiesz et al., (1980) Proc. Natl. Acad. Sci. USA, 77: 1415; Hoffman et al., (1985) Virology 147: 326; all hereby incorporated by reference.

Recently, highly sensitive PCR based assays have been developed that can detect RNA-dependent DNA polymerase in the equivalent of one to ten particles (Silver et al. (1993) Nucleic Acids Res. 21: 3593-4; U.S. Pat. No. 5,807,669). One such assay, designated as PBRT (PCR-based reverse transcriptase), has been used to detect RT activity in a variety of samples (Pyra et al. (1994) Proc. Natl. Acad. Sci. USA 51: 1544-8; Boni, et al. (1996) J. Med. Virol. 49: 23-28). This assay is 10⁶-10⁷ more sensitive than the conventional RT assay.

Other useful RT assays include, but are not limited to, one-step fluorescent probe product-enhanced reverse transcriptase assay described in Hepler, R. W., and Keller, P. M. (1998). Biotechniques 25(1), 98-106; an improved product enhanced reverse transcriptase assay described in Chang, A., Ostrove, J. M., and Bird, R. E. (1997) J Virol Methods 65(1), 45-54; an improved non-radioisotopic reverse transcriptase assay described in Nakano et al., (1994) Kansenshogaku Zasshi 68(7), 923-3 1; a highly sensitive qualitative and quantitative detection of reverse transcriptase activity as described in Yamamoto, S., Folks, T. M., and Heneine, W. (1996) J Virol Methods 61(1-2), 135-43, all references hereby incorporated by reference.

RT activity can be measured using radioactive or non-radioactive labels.

In one embodiment, 1 μl of appropriately purified DNA polymerase mutant or diluted bacterial extract (i.e., heat-treated and clarified extract of bacterial cells expressing a cloned polymerase or mutated cloned polymerase) is added to 10 μl of each nucleotide cocktail (200 μM dATP, 200 μM dGTP, 200 μM dCTP and 5 μCi/ml α-³³P dCTP and 200 μM dTTP, a RNA template, 1× appropriate buffer, followed by incubation at the optimal temperature for 30 minutes (e.g., 72° C. for Pfu DNA polymerase), for example, as described in Hogrefe et al., 2001, Methods in Enzymology, 343:91-116. Extension reactions are then quenched on ice, and 5 μl aliquots are spotted immediately onto DE81 ion-exchange filters (2.3 cm; Whatman #3658323). Unincorporated label is removed by 6 washes with 2×SCC (0.3M NaCl, 30 mM sodium citrate, pH 7.0), followed by a brief wash with 100% ethanol. Incorporated radioactivity is then measured by scintillation counting. Reactions that lack enzyme are also set up along with sample incubations to determine “total cpms” (omit filter wash steps) and “minimum cpms” (wash filters as above). Cpms bound is proportional to the amount of RT activity present per volume of bacterial extract or purified DNA polymerase.

In another embodiment, the RT activity is measured by incorporation of non-radioactive digoxigenin labeled dUTP into the synthesized DNA and detection and quantification of the incorporated label essentially according to the method described in Holtke, H.-J.; Sagner, G; Kessler, C. and Schmitz, G. (1992) Biotechniques 12, 104-113. The reaction is performed in a reaction mixture consists of the following components: 1 μg of polydA-(dT)₁₅, 33 μM of dTTP, 0.36 μM of labeled-dUTP, 200 mg/ml BSA, 10 mM Tris-HCl, pH 8.5, 20 mM KCl, 5 mM MgCl₂, 10 mM DTE and various amounts of DNA polymerase. The samples are incubated for 30 min. at 50° C., the reaction is stopped by addition of 2 μ0.5 M EDTA, and the tubes placed on ice. After addition of 8 μl 5 M NaCl and 150 μl of Ethanol (precooled to −20° C.) the DNA is precipitated by incubation for 15 min on ice and pelleted by centrifugation for 10 min at 13000×rpm and 4° C. The pellet is washed with 100 μl of 70% Ethanol (precooled to −20° C.) and 0.2 M NaCl, centrifuged again and dried under vacuum.

The pellets are dissolved in 50 μl Tris-EDTA (10 mM/0.1 mM; pH 7.5). 5 μl of the sample are spotted into a well of a nylon membrane bottomed white microwave plate (Pall Filtrationstechnik GmbH, Dreieich, FRG, product no: SM045BWP). The DNA is fixed to the membrane by baking for 10 min. at 70° C. The DNA loaded wells are filled with 100 μl of 0.45 μm-filtrated 1% blocking solution (100 mM maleic acid, 150 mM NaCl, 1% (w/v) casein, pH 7.5). All following incubation steps are done at room temperature. After incubation for 2 min. the solution is sucked through the membrane with a suitable vacuum manifold at −0.4 bar. After repeating the washing step, the wells are filled with 100 μl of a 1:10,000-dilution of Anti-digoxigenin-AP, Fab fragments (Boehringer Mannheim, FRG, no: 1093274) diluted in the above blocking solution. After incubation for 2 min. and sucking this step is repeated once. The wells are washed twice under vacuum with 200 μl each time washing-buffer 1 (100 mM maleic-acid, 150 mM NaCl, 0.3%(v/v) Tween.™. 20, pH 7.5). After washing another two times under vacuum with 200 μl each time washing-buffer 2 (10 mM Tris-HCl, 100 mM NaCl, 50 mM MgCl₂, pH 9.5) the wells are incubated for 5 min with 50 μl of CSPD™ (Boehringer Mannheim, no: 1655884), diluted 1:100 in washing-buffer 2, which serves as a chemiluminescent substrate for the alkaline phosphatase. The solution is sucked through the membrane and after 10 min incubation the RLU/s (Relative Light Unit per second) are detected in a Luminometer e.g. MicroLumat LB 96 P (EG&G Berthold, Wilbad, FRG). With a serial dilution of Taq DNA polymerase a reference curve is prepared from which the linear range serves as a standard for the activity determination of the DNA polymerase to be analyzed.

U.S. Pat. No. 6,100,039 (incorporated hereby by reference) describes another useful process for detecting reverse transcriptase activity using fluorescence polarization: the reverse transcriptase activity detection assays are performed using a Beacon™ 2000 Analyzer. The following reagents are purchased from commercial sources: fluorescein-labeled oligo dA-F (Bio.Synthesis Corp., Lewisville, Tex.), AMV Reverse Transcriptase (Promega Corp., Madison, Wis.), and Polyadenylic Acid Poly A (Pharmacia Biotech, Milwaukee, Wis.). The assay requires a reverse trancriptase reaction step followed by a fluorescence polarization-based detection step. The reverse transcriptase reactions are completed using the directions accompanying the kit. In the reaction 20 ng of Oligo (dT) were annealed to 1 μg of Poly A at 70° C. for 5 minutes. The annealed reactions are added to an RT mix containing RT buffer and dTTP nucleotides with varying units of reverse transcriptase (30, 15, 7.5, 3.8, and 1.9 Units/Rxn). Reactions are incubated at 37° C. in a water bath. 5 μl aliquots are quenched at 5, 10, 15, 20, 25, 30, 45, and 60 minutes by adding the aliquots to a tube containing 20 μl of 125 mM NaOH. For the detection step, a 75 μl aliquot of oligo dA-F in 0.5 M Tris, pH 7.5, is added to each quenched reaction. The samples are incubated for 10 minutes at room temperature. Fluorescence polarization in each sample was measured using the Beacon™ 2000 Analyzer.

Non-Conventional Nucleotides Useful According to the Invention.

There are a wide variety of non-conventional nucleotides available in the art. Any or all of them are contemplated for use with a DNA polymerase of the invention. A non-limiting list of such non-conventional nucleotides is presented in Table V.

TABLE V
Non-Conventional Nucleotides
DIDEOXYNUCLEOTIDE ANALOGS

| | |
|---|---|
| Fluorescein Labeled | Fluorophore Labeled |
| Fluorescein-12-ddCTP | Eosin-6-ddCTP |
| Fluorescein-12-ddUTP | Coumarin-5-ddUTP |
| Fluorescein-12-ddATP | Tetramethylrhodamine- |
| | 6-ddUTP |
| Fluorescein-12-ddGTP | Texas Red-5-ddATP |
| Fluorescein-N6-ddATP | LISSAMINE ™-rhodamine- |
| | 5-ddGTP |
| FAM Labeled | TAMRA Labeled |
| FAM-ddUTP | TAMRA-ddUTP |
| FAM-ddCTP | TAMRA-ddCTP |
| FAM-ddATP | TAMRA-ddATP |
| FAM-ddGTP | TAMRA-ddGTP |
| ROX Labeled | JOE Labeled |
| ROX-ddUTP | JOE-ddUTP |
| ROX-ddCTP | JOE-ddCTP |
| ROX-ddATP | JOE-ddATP |
| ROX-ddGTP | JOE-ddGTP |
| R6G Labeled | R110 Labeled |
| R6G-ddUTP | R110-ddUTP |
| R6G-ddCTP | R110-ddCTP |
| R6G-ddATP | R110-ddATP |
| R6G-ddGTP | R110-ddGTP |
| BIOTIN Labeled | DNP Labeled |
| Biotin-N6-ATP | DNP-N6-ddATP |

DEOXYNUCLEOTIDE ANALOGS

| | |
|---|---|
| TTP Analogs | dATP-Analogs |
| Fluorescein-12-dUTP | Coumarin-5-dATP |
| Coumarin-5-dUTP | Diethylaminocoumarin- |
| | 5-dATP |
| Tetramethylrhodamine-6-dUTP | Fluorescein-12-dATP |
| Tetraethylrhodamine-6-dUTP | Fluorescein Chlorotria- |
| | zinyl-4-dATP |
| Texas Red-5-dUTP | LISSAMINE ™-rhodamine- |
| | 5-dATP |
| LISSAMINE ™-rhodamine-5-dUTP | Naphthofluorescein- |
| | 5-dATP |
| Naphthofluorescein-5-dUTP | Pyrene-8-dATP |
| Fluorescein Chlorotriazinyl-4-dUTP | Tetramethylrhodamine- |
| | 6-dATP |
| Pyrene-8-dUTP | Texas Red-5-dATP |
| Diethylaminocoumarin-5-dUTP | DNA-N6-dATP |
| Amino allyl dUTP | Biotin-N6-dATP |
| dCTP Analogs | dGTP Analogs |
| Coumarin-5-dCTP | Coumarin-5-dGTP |
| Fluorescein-12-dCTP | Fluorescein-12-dGTP |
| Tetramethylrhodamine-6-dCTP | Tetramethylrhodamine- |
| | 6-dGTP |
| Texas Red-5-dCTP | Texas Red-5-dGTP |
| LISSAMINE ™-rhodamine-5-dCTP | LISSAMINE ™-rhodamine- |
| | 5-dGTP |
| Naphthofluorescein-5-dCTP | |
| Fluorescein Chlorotriazinyl-4-dCTP | |
| Pyrene-8-dCTP | Diethylaminocoumarin- |
| | 5-dCTP |
| Fluorescein-N4-dCTP | |
| Biotin-N4-dCTP | |
| DNP-N4-dCTP | |
| Amino-allyl dCTP | |
| amino hexyl dCTP | |

RIBONUCLEOTIDE ANALOGS

| | |
|---|---|
| CTP Analogs | UTP Analogs |
| Coumarin-5-CTP | Fluorescein-12-UTP |
| Fluorescein-12-CTP | Coumarin-5-UTP |
| Tetrainethylrhodainine-6-CTP | Tetramethylrhodamine- |
| | 6-UTP |
| Texas Red-5-CTP | Texas Red-5-UTP |
| LISSAMINE ™-rhodamine-5-CTP | LISSAMINE ™-5-UTP |
| Naphthofluorescein-5-CTP | Naphthofluorescein- |
| | 5-UTP |
| Fluorescein Chlorotriazinyl-4-CTP | Fluorescein Chlorotria- |
| | zinyl-4-UTP |
| Pyrene-8-CTP | Pyrene-8-UTP |
| Fluorescein-N4-CTP | Amino allyl UTP |
| Biotin-N4-CTP | |
| Amino allyl CTP | |
| ATP Analogs | |
| Coumarin-5-ATP | |
| Fluorescein-12-ATP | |
| Tetramethylrhodamine-6-ATP | |
| Texas Red-5-ATP | |
| LISSAMINE ™-rhodamine-5-ATP | |
| Fluorescein-N6-ATP | |
| Biotin-N6-ATP | |
| DNP-N6-ATP | |

Additional non-conventional nucleotides useful according to the invention include, but are not limited to 7-deaza-dATP, 7-deaza-dGTP, 5′-methyl-2′-deoxycytidine-5′-triphosphate and DIG-labeled nucleotides. Further non-conventional nucleotides or variations on those listed above are taught in U.S. Pat. No. 6,383,749B2, Wright & Brown, 1990, and Pharmacol. Ther. 47: 447 all of which are herein incorporated by reference. It is specifically noted that ribonucleotides qualify as non-conventional nucleotides, since ribonucleotides are not generally incorporated by DNA polymerases.

The amino allyl modified nucleotides, e.g., amino allyl dUTP, amino allyl UTP, amino hexyl modified nucleotides, e.g., amino hexyl dCTP, can be coupled to any florescent dye containing a NHS- or STP-ester leaving group. These fluorescent dyes include the those in the ARES Alexa Fluor DNA labeling kits (Molecular Probes, Eugene, Oreg.; Cat.# A-21675, 21674, 21665, 21666, 21667, 21677, 21668, 21669, 21676) and CYDYE mono-Reactive Dye 5-Pack (Amersham Pharmacia Biotech; Cat.# PA23001, 23501, 25001, 25501).

Expression of Wild-Type or Mutant Enzymes According to the Invention

Methods known in the art may be applied to express and isolate the mutated forms of DNA polymerase according to the invention. The methods described here can be also applied for the expression of wild-type enzymes useful in the invention. Many bacterial expression vectors contain sequence elements or combinations of sequence elements allowing high level inducible expression of the protein encoded by a foreign sequence. For example, as mentioned above, bacteria expressing an integrated inducible form of the T7 RNA polymerase gene may be transformed with an expression vector bearing a mutated DNA polymerase gene linked to the T7 promoter. Induction of the T7 RNA polymerase by addition of an appropriate inducer, for example, isopropyl-β-D-thiogalactopyranoside (IPTG) for a lac-inducible promoter, induces the high level expression of the mutated gene from the T7 promoter.

Appropriate host strains of bacteria may be selected from those available in the art by one of skill in the art. As a non-limiting example, E. coli strain BL-21 is commonly used for expression of exogenous proteins since it is protease deficient relative to other strains of E. coli. BL-21 strains bearing an inducible T7 RNA polymerase gene include WJ56 and ER2566 (Gardner & Jack, 1999, supra). For situations in which codon usage for the particular polymerase gene differs from that normally seen in E. coli genes, there are strains of BL-21 that are modified to carry tRNA genes encoding tRNAs with rarer anticodons (for example, argu, ileY, leuW, and proL tRNA genes), allowing high efficiency expression of cloned protein genes, for example, cloned archaeal enzyme genes (several BL21-CODON PLUSTM cell strains carrying rare-codon tRNAs are available from Stratagene, for example).

There are many methods known to those of skill in the art that are suitable for the purification of a mutant DNA polymerase of the invention. For example, the method of Lawyer et al. (1993, PCR Meth. & App. 2: 275) is well suited for the isolation of DNA polymerases expressed in E. coli, as it was designed originally for the isolation of Taq polymerase. Alternatively, the method of Kong et al. (1993, J. Biol. Chem. 268: 1965, incorporated herein by reference) may be used, which employs a heat denaturation step to destroy host proteins, and two column purification steps (over DEAE-Sepharose and heparin-Sepharose columns) to isolate highly active and approximately 80% pure DNA polymerase. Further, DNA polymerase mutants may be isolated by an ammonium sulfate fractionation, followed by Q Sepharose and DNA cellulose columns, or by adsorption of contaminants on a HiTrap Q column, followed by gradient elution from a HiTrap heparin column.

In one embodiment, the Pfu mutants are expressed and purified as described in U.S. Pat. No. 5,489,523, hereby incorporated by reference in its entirety.

In another embodiment, the JDF-3 mutants are expressed and purified as described in U.S. patent application Ser. No. 09/896,923, hereby incorporated by reference in its entirety.

Kits

The invention herein also contemplates a kit format which comprises a package unit having one or more containers of the subject composition and in some embodiments including containers of various reagents used for polynucleotide synthesis, including RT, RT-PCR, RNA amplification, cDNA labelling and RNA labelling.

It is contemplated that the kits of the present invention find use for methods including, but not limited to, reverse transcribing template RNA for the construction of cDNA libraries, for the reverse transcription of RNA for differential display PCR, for RT-PCR identification of target RNA in a sample suspected of containing the target RNA, for RNA amplification, for the generation of sense and anti-sense RNA, for labeling nucleic acids for use in microarray and in situ assays, and for other methods in which RNA can be used. In some embodiments, the RT, RT-PCR, RNA amplification and RNA labeling kits comprise the essential reagents required for the method of reverse transcription. For example, in some embodiments, the kit includes a vessel containing a polymerase with increased RT activity. In some embodiments, the concentration of polymerase ranges from about 0.1 to 100 u/μl; in other embodiments, the concentration is about 5 u/μl. In some embodiments, kits for reverse transcription also include a vessel containing a RT reaction buffer. Preferably, these reagents are free of contaminating RNase activity. In other embodiments of the present invention, reaction buffers comprise a buffering reagent in a concentration of about 5 to 15 mM (preferably about 10 mM Tris-HCl at a pH of about 7.5 to 9.0 at 25° C.), a monovalent salt in a concentration of about 20 to 100 mM (preferably about 50 mM NaCl or KCl), a divalent cation in a concentration of about 1.0 to 10.0 mM (preferably MgCl₂), dNTPs in a concentration of about 0.05 to 3.0 mM each (preferably about 0.2 mM each), and a surfactant in a concentration of about 0.001 to 1.0% by volume (preferably about 0.01% to 0.1%). In some embodiments the kits include non-conventional nucleotides in a concentration of about 0.05 to 3.0 mM. Preferably, the non-conventional nucleotide is an amino-allyl modified nucleotide. In some embodiments, a purified RNA standard set is provided in order to allow quality control and for comparison to experimental samples. In some embodiments, the kit is packaged in a single enclosure including instructions for performing the assay methods (e.g., reverse transcription, RT-PCR, RNA amplification, labeling). In some embodiments, the reagents are provided in containers and are of strength suitable for direct use or use after dilution.

The composition or kit of the present invention may further comprise compounds for improving product yield, processivity and specificity of RT-PCR such as DMSO (preferably about 20%), formamide, betaine, trehalose, low molecular weight amides, sulfones or a PCR enhancing factor (PEF). DMSO is preferred.

The composition or kit of the present invention may further comprise a DNA binding protein, such as gene 32 protein from bacteriophage T4 (WO 00/55307, incorporated herein by reference), and the E. coli SSB protein. Other protein additives can include Archaeal PCNA, RNAse H, an exonuclease, an RNA polymerase or another reverse transcriptase. The kit can also comprise an Family B DNA polymerase LYP mutant (e.g., L408 mutant of JDF-3 polymerase, L409 mutant of Pfu DNA polymerase) fusion in which the DNA polymerase is fused, for example, to Ncp7, recA, Archaeal sequence non-specific double stranded DNA binding proteins (e.g., Sso7d from Sulfolobus solfactaricus, WO 01/92501, incorporated herein by reference), or helix-hairpin-helix domains from topoisomerase V (Pavlov et al., PNAS, 2002).

The composition or kit may also contain one or more of the following items: polynucleotide precursors, non-conventional nucleotides, fluorescent labels, primers, buffers, instructions, and controls. Kits may include containers of reagents mixed together in suitable proportions for performing the methods in accordance with the invention. Reagent containers preferably contain reagents in unit quantities that obviate measuring steps when performing the subject methods.

Application in Amplification Reactions

Reverse transcription of an RNA template into cDNA is an integral part of many techniques used in molecular biology. Accordingly, the reverse transcription procedures, compositions, and kits provided in the present invention find a wide variety of uses. For example, it is contemplated that the reverse transcription procedures and compositions of the present invention are utilized to produce cDNA inserts for cloning into cDNA library vectors (e.g., lambda gt10 [Huynh et al., In DNA Cloning Techniques: A Practical Approach, D. Glover, ed., IRL Press, Oxford, 49, 1985], lambda gt11 [Young and Davis, Proc. Nat'l. Acad. Sci., 80:1194, 1983], pBR322 [Watson, Gene 70:399-403, 1988], pUC19 [Yarnisch-Pyrron et al., Gene 33:103-119, 1985], and M13 [Messing et al., Nucl. Acids. Res. 9:309-321, 1981]). The present invention also finds use for identification of target RNAs in a sample via RT-PCR (e.g., U.S. Pat. No. 5,322,770, incorporated herein by reference). Additionally, the present invention finds use in providing cDNA templates for techniques such as differential display PCR (e.g., Liang and Pardee, Science 257(5072):967-71 (1992), FISH analysis (fluorescence in situ hybridization), and microarray and other hybridization techniques. The DNA polymerase with increased RT activity, compositions or kits comprising such polymerase can be applied in any suitable applications, including, but not limited to the following examples.

1. Reverse Transcription

The present invention contemplates the use of thermostable DNA polymerase for reverse transcription reactions. Accordingly, in some embodiments of the present invention, thermostable DNA polymerases having increased RT activity are provided. In some embodiments, the thermostable DNA polymerase is selected from the DNA polymerases listed in Tables II-IV, for example, a Pfu or a JDF-3 DNA polymerase.

In some embodiments of the present invention, where a DNA polymerase with increased RT activity is utilized to reverse transcribe RNA, the reverse transcription reaction is conducted at about 50° C. to 80° C., preferably about 60° C. to 75° C. Optimal reaction temperature for each DNA polymerase is know in the art and may be relied upon as the optimal temperature for the mutant DNA polymerases of the present invention. Preferred conditions for reverse transcription are 1× MMLV RT buffer (50 mM Tris pH 8.3, 75 mM KCl, 10 mM DTT, 3 mM MgCl₂), containing 20% DMSO.

In still further embodiments, reverse transcription of an RNA molecule by a DNA polymerase with increased RT activity results in the production of a cDNA molecule that is substantially complementary to the RNA molecule. In other embodiments, the DNA polymerase with increased RT activity then catalyzes the synthesis of a second strand DNA complementary to the cDNA molecule to form a double stranded DNA molecule. In still further embodiments of the present invention, the DNA polymerase with increased RT activity catalyzes the amplification of the double stranded DNA molecule in a PCR as described below. In some embodiments, PCR is conducted in the same reaction mix as the reverse transcriptase reaction (i.e., a single tube reaction is performed). In other embodiments, PCR is performed in a separate reaction mix on an aliquot removed from the reverse transcription reaction (i.e., a two tube reaction is performed).

In some embodiment, the DNA polymerase mutants of the invention can be used to generate labeled cDNA, e.g., for use on a microarray. In one embodiment the DNA polymerase mutants of the invention incorporate a non-conventional nucleotide, e.g., amino allyl dUTP, into the synthesized strand, e.g., cDNA, sense RNA or anti-sense RNA, generating a modified nucleic acid. In a further embodiment a detectable label, e.g., fluorescent label, coupling step follows the incorporation of the amino allyl nucleotide. A fluorescent coupling step results in the attachment of a fluorescent dye, e.g., Cy3, Cy5 etc., to the non-conventional nucleotide. Such techniques are routine in the art and can be found in the product literature of FAIRPLAY microarray labeling kit (Stratagene, La Jolla, Calif.; Cat.# 252002), Manduchi et al. Physiol Genomics:10:169-179 (Jun. 18, 2002) and http://cmgm.stanford.edu/pbrown/protocols, all incorporated herein by reference. In an alternative embodiment the DNA polymerase mutants of the invention incorporate a non-conventional nucleotide that is coupled to a detectable label.

In an alternative embodiment a modified nucleic acid is generated by using a DNA polymerase of the current invention to extend a primer, e.g., oligo dT, sequence specific primer, that contains at least one non-conventional nucleotide. It is contemplated that DNA polymerase mutants as described herein would have the advantage of more efficient labeling or more uniform incorporation of labeled nucleotides relative to wild-type enzymes.

2. RT-PCR and PCR

The DNA polymerase with increased RT activity of the present invention is useful for RT-PCR because the reverse transcription reaction may be conducted in a temperature that is compatible with PCR amplification. Another advantage is the possibility of using the same enzyme for cDNA synthesis and PCR amplification. Further, the high temperature at which the thermostable Family B DNA polymerases function allows complete denaturation of RNA secondary structure, thereby enhancing processivity. The present invention contemplates single-reaction RT-PCR wherein reverse transcription and amplification are performed in a single, continuous procedure. The RT-PCR reactions of the present invention serve as the basis for many techniques, including, but not limited to diagnostic techniques for analyzing mRNA expression, synthesis of cDNA libraries, rapid amplification of cDNA ends (i.e., RACE) and other amplification-based techniques known in the art. Any type of RNA may be reverse transcribed and amplified by the methods and reagents of the present invention, including, but not limited to RNA, rRNA, and mRNA. The RNA may be from any source, including, but not limited to, bacteria, viruses, fungi, protozoa, yeast, plants, animals, blood, tissues, and in vitro synthesized nucleic acids.

The DNA polymerase with increased RT activity of the present invention provides suitable enzymes for use in the PCR. The PCR process is described in U.S. Pat. Nos. 4,683,195 and 4,683,202, the disclosures of which are incorporated herein by reference. In some embodiments, at least one specific nucleic acid sequence contained in a nucleic acid or mixture of nucleic acids is amplified to produce double stranded DNA. Primers, template, nucleoside triphosphates, the appropriate buffer and reaction conditions, and polymerase are used in the PCR process, which involves denaturation of target DNA, hybridization of primers and synthesis of complementary strands. The extension product of each primer becomes a template for the production of the desired nucleic acid sequence. If the polymerase employed in the PCR is a thermostable enzyme, then polymerase need not be added after each denaturation step because heat will not destroy the polymerase activity. Use of thermostable DNA polymerase with increased RT activity allows repetitive heating/cooling cycles without the requirement of fresh enzyme at each cooling step. This represents a major advantage over the use of mesophilic enzymes (e.g., Klenow), as fresh enzyme must be added to each individual reaction tube at every cooling step.

In some embodiments of the present invention, primers for reverse transcription also serve as primers for amplification. In other embodiments, the primer or primers used for reverse transcription are different than the primers used for amplification. In some embodiments, the primers contain an RNA promoter element. In further embodiments the primers include at least one non-conventional nucleotide. In some embodiments, more than one RNA in a mixture of RNAs may be amplified or detected by RT-PCR. In other embodiments, multiple RNAs in a mixture of RNAs may be amplified in a multiplex procedure (e.g., U.S. Pat. No. 5,843,660, incorporated herein by reference).

In addition to the subject enzyme mixture, one of ordinary skill in the art may also employ other PCR parameters to increase the fidelity of synthesis/amplification reaction. It has been reported PCR fidelity may be affected by factors such as changes in dNTP concentration, units of enzyme used per reaction, pH, and the ratio of Mg²⁺ to dNTPs present in the reaction. The fidelity of the reverse transcription step can be increased by adding an exonuclease to the reverse transcription, or the exonuclease activity of polymerase mutants described herein (e.g., L408 mutants of JDF-3 polymerase, L409 mutants of Pfu polymerase) could be used to excise mispaired nucleotides in the DNA/RNA duplex.

Mg²⁺ concentration affects the annealing of the oligonucleotide primers to the template DNA by stabilizing the primer-template interaction, it also stabilizes the replication complex of polymerase with template-primer. It can therefore also increase non-specific annealing and produce undesirable PCR products (giving multiple bands on a gel). When non-specific amplification occurs, Mg²⁺ may need to be lowered or EDTA can be added to chelate Mg²⁺ to increase the accuracy and specificity of the amplification.

Other divalent cations such as Mn²⁺, or Co²⁺ can also affect DNA polymerization. Suitable cations for each DNA polymerase are known in the art (e.g., in DNA Replication 2^{nd} edition, supra). Divalent cation is supplied in the form of a salt such MgCl₂, Mg(OAc)₂, MgSO₄, MnCl₂, Mn(OAc)₂, or MnSO₄. Usable cation concentrations in a Tris-HCl buffer are for MnCl₂ from 0.5 to 7 mM, preferably, between 0.5 and 2 mM, and for MgCl₂ from 0.5 to 10 mM. Usable cation concentrations in a Bicine/KOAc buffer are from 1 to 20 mM for Mn(OAc)₂, preferably between 2 and 5 mM.

Monovalent cation required by DNA polymerase may be supplied by the potassium, sodium, ammonium, or lithium salts of either chloride or acetate. For KCl, the concentration is between 1 and 200 mM, preferably the concentration is between 40 and 100 mM, although the optimum concentration may vary depending on the polymerase used in the reaction.

Deoxyribonucleotide triphosphates (dNTPs) are added as solutions of the salts of dATP, dCTP, dGTP and dTTP, such as disodium or lithium salts. The dNTPs can also include one or more non-conventional nucleotides. In the present methods, a final concentration in the range of 1 μM to 2 mM each is suitable, and 100-600 μM is preferable, although the optimal concentration of the nucleotides may vary in the PCR reaction depending on the total dNTP and divalent metal ion concentration, and on the buffer, salts, particular primers, and template. For longer products, i.e., greater than 1500 bp, 500 μM each dNTP may be preferred when using a Tris-HCl buffer.

dNTPs chelate divalent cations, therefore amount of divalent cations used may need to be changed according to the dNTP concentration in the reaction. Excessive amount of dNTPs (e.g., larger than 1.5 mM) can increase the error rate and possibly inhibit DNA polymerases. Lowering the dNTP (e.g., to 10-50 μM) may therefore reduce error rate. PCR reaction for amplifying larger size template may need more dNTPs.

One suitable buffering agent is Tris-HCl, preferably pH 8.3, although the pH may be in the range 8.0-8.8. The Tris-HCl concentration is from 5-250 mM, although 10-100 mM is most preferred. Other preferred buffering agents are Bicine-KOH and Tricine.

Denaturation time may be increased if template GC content is high. Higher annealing temperature may be needed for primers with high GC content or longer primers. Gradient PCR is a useful way of determining the annealing temperature. Extension time should be extended for larger PCR product amplifications. However, extension time may need to be reduced whenever possible to limit damage to enzyme.

The number of cycles can be increased if the number of template DNA molecules is very low, and decreased if a higher amount of template DNA is used.

PCR enhancing factors may also be used to improve efficiency of the amplification. As used herein, a “PCR enhancing factor” or a “Polymerase Enhancing Factor” (PEF) refers to a complex or protein possessing polynucleotide polymerase enhancing activity (Hogrefe et al., 1997, Strategies 10:93-96; and U.S. Pat. No. 6,183,997, both of which are incorporated herein by reference). For Pfu DNA polymerase, PEF comprises either P45 in native form (as a complex of P50 and P45) or as a recombinant protein. In the native complex of Pfu P50 and P45, only P45 exhibits PCR enhancing activity. The P50 protein is similar in structure to a bacterial flavoprotein. The P45 protein is similar in structure to dCTP deaminase and dUTPase, but it functions only as a dUTPase converting dUTP to dUMP and pyrophosphate. PEF, according to the present invention, can also be selected from the group consisting of: an isolated or purified naturally occurring polymerase enhancing protein obtained from an archeabacteria source (e.g., Pyrococcus furiosus); a wholly or partially synthetic protein having the same amino acid sequence as Pfu P45, or analogs thereof possessing polymerase enhancing activity; polymerase-enhancing mixtures of one or more of said naturally occurring or wholly or partially synthetic proteins; polymerase-enhancing protein complexes of one or more of said naturally occurring or wholly or partially synthetic proteins; or polymerase-enhancing partially purified cell extracts containing one or more of said naturally occurring proteins (U.S. Pat. No. 6,183,997, supra). The PCR enhancing activity of PEF is defined by means well known in the art. The unit definition for PEF is based on the dUTPase activity of PEF (P45), which is determined by monitoring the production of pyrophosphate (PPi) from dUTP. For example, PEF is incubated with dUTP (10 mM dUTP in 1× cloned Pfu PCR buffer) during which time PEF hydrolyzes dUTP to dUMP and PPi. The amount of PPi formed is quantitated using a coupled enzymatic assay system that is commercially available from Sigma (#P7275). One unit of activity is functionally defined as 4.0 nmole of PPi formed per hour (at 85° C.).

Other PCR additives may also affect the accuracy and specificity of PCR reaction. EDTA less than 0.5 mM may be present in the amplification reaction mix. Detergents such as Tween-20™ and Nonidet™ P-40 are present in the enzyme dilution buffers. A final concentration of non-ionic detergent approximately 0.1% or less is appropriate, however, 0.01-0.05% is preferred and will not interfere with polymerase activity. Similarly, glycerol is often present in enzyme preparations and is generally diluted to a concentration of 1-20% in the reaction mix. Glycerol (5-10%), formamide (1-5%) or DMSO (2-20%) can be added in PCR for template DNA with high GC content or long length (e.g., >1 kb). DMSO, preferably at about 20%, can be added for the cDNA synthesis step using mutant Family B polymerases described herein. These additives change the Tₘ (melting temperature) of primer-template hybridization reaction and the thermostability of the polymerase enzyme. BSA (up to 0.8 μg/μl) can improve the efficiency of the PCR reaction. Betaine (0.5-2M) is also useful for PCR of long templates or those with a high GC content. Tetramethylammonium chloride (TMAC, >50 mM), Tetraethylammonium chloride (TEAC), and Trimethlamine N-oxide (TMANO) may also be used. Test PCR reactions may be performed to determine optimum concentration of each additive mentioned above.

LYP motif mutants as described herein (e.g., L408 mutants of JDF-3 polymerase, L409 mutants of Pfu polymerase) can be used for cDNA synthesis and for PCR amplification, however, such polymerase mutants can also be used in a mixture or blend with one or more other enzymes used for PCR, e.g., E. Coli DNA polymerase, Klenow, Exo− Pfu V93, Exo− Pfu or Pfu DNA polymerase for amplification with enhanced fidelity.

The invention provides for additives including, but not limited to antibodies (for hot start PCR) and ssb (higher specificity). The invention also contemplates mutant Family B DNA polymerases in combination with Family B accessory factors, for example as described in U.S. Pat. No. 6,333,158 (e.g., F7, PFU-RFC and PFU-RFCLS described therein), and WO 01/09347 (e.g., Archaeal PCNA, Archaeal RFC, Archaeal RFC-p55, Archaeal RFC-p38, Archaeal RFA, Archaeal MCM, Archaeal CDC6, Archaeal FEN-1, Archaeal ligase, Archaeal dUTPase, Archaeal helicases 2-8 and Archaeal helicase dna2 described therein), both of which are incorporated herein by reference in their entireties. Further additives include exonucleases such as Pfu G387P to increase fidelity.

Various specific PCR amplification applications are available in the art (for reviews, see for example, Erlich, 1999, Rev Immunogenet., 1: 127-34; Prediger 2001, Methods Mol. Biol. 160:49-63; Jurecic et al., 2000, Curr. Opin. Microbiol. 3:316-21; Triglia, 2000, Methods Mol. Biol. 130:79-83; MaClelland et al., 1994, PCR Methods Appl. 4:S66-81; Abramson and Myers, 1993, Current Opinion in Biotechnology 4:41-47; each of which is incorporated herein by references).

The subject invention can be used in RT-PCR or PCR applications, where the PCR applications include, but are not limited to, i) hot-start PCR which reduces non-specific amplification; ii) touch-down PCR which starts at high annealing temperature, then decreases annealing temperature in steps to reduce non-specific PCR product; iii) nested PCR which synthesizes more reliable product using an outer set of primers and an inner set of primers; iv) inverse PCR for amplification of regions flanking a known sequence. In this method, DNA is digested, the desired fragment is circularized by ligation, then PCR using primer complementary to the known sequence extending outwards; v) AP-PCR (arbitrary primed)/RAPD (random amplified polymorphic DNA). These methods create genomic fingerprints from species with little-known target sequences by amplifying using arbitrary oligonucleotides; vi) RT-PCR which uses RNA-directed DNA polymerase (e.g., reverse transcriptase) to synthesize cDNAs which is then used for PCR. This method is extremely sensitive for detecting the expression of a specific sequence in a tissue or cells. It may also be use to quantify mRNA transcripts; vii) RACE (rapid amplification of cDNA ends). This is used where information about DNA/protein sequence is limited. The method amplifies 3′ or 5′ ends of cDNAs generating fragments of cDNA with only one specific primer each (plus one adaptor primer). Overlapping RACE products can then be combined to produce full length cDNA; viii) DD-PCR (differential display PCR) which is used to identify differentially expressed genes in different tissues. First step in DD-PCR involves RT-PCR, then amplification is performed using short, intentionally nonspecific primers; ix) Multiplex-PCR in which two or more unique targets of DNA sequences in the same specimen are amplified simultaneously. One DNA sequence can be use as control to verify the quality of PCR; x) Q/C-PCR (Quantitative comparative) which uses an internal control DNA sequence (but of different size) which compete with the target DNA (competitive PCR) for the same set of primers; xi) Recusive PCR which is used to synthesize genes. Oligonucleotides used in this method are complementary to stretches of a gene (>80 bases), alternately to the sense and to the antisense strands with ends overlapping (˜20 bases); xii) Asymmetric PCR; xiii) In Situ PCR; xiv) Site-directed PCR Mutagenesis.

In some embodiment, the DNA polymerase mutants of the invention can be used to generate labeled DNA. In one embodiment the DNA polymerase mutants of the invention incorporate a non-conventional nucleotide, e.g., amino allyl dUTP, into the synthesized strand, e.g., cDNA, generating a modified nucleic acid. In a further embodiment a detectable label, e.g., fluorescent label, coupling step follows the incorporation of the amino allyl nucleotide. A fluorescent coupling step results in the attachment of a fluorescent dye, e.g., Cy3, Cy5 etc., to the non-conventional nucleotide. Such techniques are routine in the art and can be found in the product literature of FAIRPLAY microarray labeling kit (Stratagene, La Jolla, Calif.; Cat.# 252002), Manduchi et al. Physiol Genomics:10:169-179 (Jun. 18, 2002) and http://cmgm.stanford.edu/pbrown/protocols, all incorporated herein by reference. In an alternative embodiment the DNA polymerase mutants of the invention incorporate a non-conventional nucleotide that is coupled to a detectable label.

In some embodiment a modified nucleic acid is generated by using a DNA polymerase, e.g., DNA polymerase of the current invention, Pfu RT, to extend a primer, e.g., oligo dT, sequence specific primer, that contains at least one non-conventional nucleotide.

3. Amplification of RNA Using Promoter Sequence

The DNA polymerase with increased RT activity of the present invention are useful for RNA amplification utilizing an RNA promoter. The RNA promoter based amplification reactions of the present invention serve as the basis for many techniques, including, but not limited to diagnostic techniques for analyzing mRNA expression, synthesizing cDNA libraries and other amplification-based techniques known in the art. Any type of RNA may be utilized including, but not limited to RNA, rRNA, and mRNA. The RNA may be from any source, including, but not limited to, bacteria, viruses, fingi, protozoa, yeast, plants, animals, blood, tissues and in vitro synthesized nucleic acids.

The DNA polymerases with increased RT activity of the present invention provide suitable enzymes for use in RNA promoter based amplification reactions. The RNA promoter based amplification reactions are described in U.S. Pat. Nos. 5,545,522 and 6,027,913, the disclosures of which are herein incorporated by reference. In some embodiments, at least one specific nucleic acid sequence contained in a nucleic acid or mixture of nucleic acids is amplified to produce an antisense RNA. The process described in U.S. Pat. No. 5,545,522 utilizes an RNA polymerase promoter incorporated at the 5′ end of the primer complex.

In one general embodiment of the present invention, cDNA strands are synthesized from a collection of mRNA's using an oligonucleotide primer complex, i.e., a primer linked to an RNA promoter region. If the target mRNA is the entire mRNA population, then the primer can be a polythymidylate region (e.g., about 5 to 20, preferably about 10 to 15 T residues), which will bind with the poly(A) tail present on the 3′ terminus of each mRNA. The primer may also be an anchored primer with the sequence (5′-T₍₅₋₂₀₎ VN-3′), wherein V is G, A or C and N is G, A, C, or T. Alternatively, if only a preselected mRNA is to be amplified, then the primer will be substantially complementary to a section of the chosen mRNA, typically at the 3′ terminus. The promoter region is located upstream of the primer at the 5′ terminus in an orientation permitting transcription with respect to the mRNA population utilized. This will usually, but not always, mean that the promoter DNA sequence operably linked to the primer is the complement to the functional promoter sequence. When the second cDNA strand is synthesized, the promoter sequence will be in correct orientation in that strand to initiate RNA synthesis using that second cDNA strand as a template. Preferably, the promoter region is derived from a prokaryote, and more preferably from the group consisting of SP6, T3 and T7 phages (Chamberlin and Ryan, in The Enzymes, ed. P. Boyer (Academic Press, New York) pp. 87-108 (1982), which is incorporated herein by reference). A preferred promoter region is the sequence from the T7 phage that corresponds to its RNA polymerase binding site (5′ TAA TAC GAC TCA CTA TAG GG 3′).

Once the oligonucleotide primer and linked promoter region hybridize to the mRNA, a first cDNA strand is synthesized. This first strand of cDNA is preferably produced through the process of reverse transcription. The reverse transcription is performed by the DNA polymerases of the current invention.

The second strand cDNA, creating double-stranded (ds) cDNA, can be synthesized by a variety of means, but preferably with the addition of RNase H and DNA polymerase. RNase assists breaking the RNA/first strand cDNA hybrid, and DNA polymerase synthesizes a complementary DNA strand from the template DNA strand. The second strand is generated as deoxynucleotides are added to the 3′ terminus of the growing strand. As the growing strand reaches the 5′ terminus of the first strand DNA, the complementary promoter region of the first strand will be copied into the double stranded promoter sequence in the desired orientation.

Another means for synthesizing the second strand cDNA is by removing or nicking the RNA of the RNA/first strand cDNA hybrid with RNase H. A second primer is incubated with the first strand cDNA. The second primer can have one or more degenerate bases at the 3′ end that bind to a preselected target sequence. The same primer may include a preselected nucleotide sequence at the 5′ end, e.g., RNA polymerase promoter sequence. The second primer may include one or more fixed nucleotides at the 3′ end that bind the target sequence.

Thereafter, cDNA is transcribed into anti-sense RNA (aRNA) by introducing an RNA polymerase capable of binding to the promoter region. The second strand of cDNA is transcribed into aRNA, which is the complement of the initial mRNA population. Amplification occurs because the polymerase repeatedly recycles on the template (i.e., reinitiates transcription from the promoter region).

The RNA polymerase used for the transcription must be capable of operably binding to the particular promoter region employed in the primer complex. A preferred RNA polymerase is that found in bacteriophages, in particular T3 and T7 phages. Substantially any polymerase/promoter combination can be used, however, provided the polymerase has specificity for that promoter in vitro sufficient to initiate transcription.

In one embodiment the RNA polymerase incorporates one or more non-conventional nucleotides into the aRNA producing a modified nucleic acid. In a further embodiment the modified nucleic acid molecule is coupled to a detectable label, e.g., fluorescent dye. In an alternative embodiment the non-conventional nucleotide is coupled to a detectable label at the time of nucleotide incorporation.

In some embodiments, at least one specific nucleic acid sequence contained in a nucleic acid or mixture of nucleic acids is amplified to produce a sense RNA. This process is similar to that described above but results in sense RNA and utilizes two different primer sets. The method is performed based on a modification of the method described in U.S. Pat. No. 6,027,913, which is herein incorporated by reference. The method is particularly described in U.S. Pat. No. 6,027,913 column 21, line 59 to column 22, line 19. First, cDNA synthesis is performed from a collection of mRNA's using an oligonucleotide primer complex using oligo(dT) or an mRNA specific oligonucleotide primer. The synthesis is performed using the DNA polymerase with increased RT activity of the present invention. Secondly a PCR reaction is performed where one or both of the oligonucleotide primers contain a promoter attached to a sequence complementary to the region to be amplified. The promoter region is derived from a prokaryote and preferably from the group consisting of SP6, T3 and T7. Finally, a transcription reaction is performed with an RNA polymerase specific for the phage promoter.

In one embodiment, the RNA polymerase can be used for labeling RNA, e.g., for use on a microarray. In another embodiment the RNA polymerase incorporate a non-conventional nucleotide, e.g., amino allyl UTP, into the synthesized strand, e.g., sense or anti-sense RNA. In a further embodiment a detectable label, e.g., fluorescent label, coupling step follows the incorporation of the amino allyl nucleotide. A fluorescent coupling step results in the attachment of a fluorescent dye, e.g., Cy3, Cy5 etc., to the non-conventional nucleotide. The coupling reactions are routine in the art and can be found in the product literature of FAIRPLAY microarray labeling kit (Stratagene, La Jolla, Calif.; Cat. # 252002), Manduchi et al. Physiol Genomics:10:169-179 (Jun. 18, 2002) and http://cmgm.stanford.edu/pbrown/protocols, all incorporated herein by reference.

It should be understood that this invention is not limited to any particular amplification system. As other systems are developed, those systems may benefit by practice of this invention.

### EXAMPLES

### Example 1

### Construction of exo− and exo+ JDF-3 and Pfu DNA Polymerase Mutants that Possess Reverse Transcriptase Activity

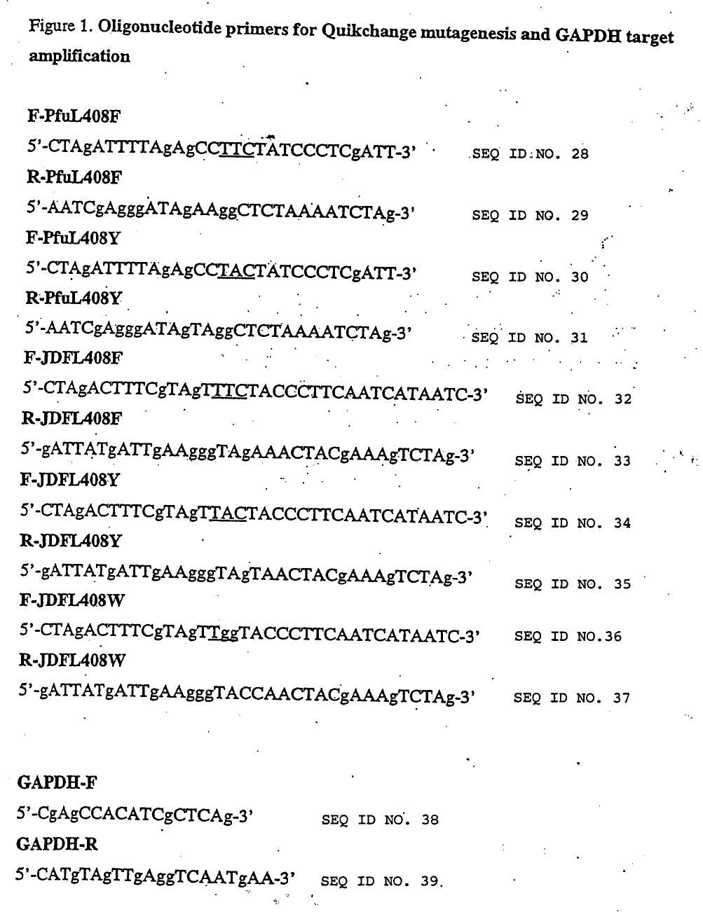

Wild-type (exo⁺) JDF-3 DNA polymerase and JDF-3 DNA polymerase substantially lacking 3′-5′ exonuclease activity (exo⁻) were prepared as described in U.S. patent application Ser. No. 09/896,923. Point mutations phenylalanine (F), tyrosine (Y), and tryptophan (W) were introduced at leucine (L) 409 of exo⁻ and exo⁺ Pfu and at L408 of exo⁻ and exo⁺ JDF-3 DNA polymerases using the Quikchange site directed mutagenesis kit (Stratagene). With the Quikchange kit, point mutations were introduced using a pair of mutagenic primers (FIG. 1). Clones were sequenced to identify the incorporated mutations. Construction of JDF-3 L408H was described previously (see patent application WO 0132887, incorporated herein by reference).

### Example 2

### Preparation of Bacterial Extracts Containing Mutant JDF-3 and Pfu DNA Polymerases

Plasmid DNA was purified with the StrataPrep® Plasmid Miniprep Kit (Stratagene), and used to transform BL26-CodonPlus-RIL cells. Ampicillin resistant colonies were grown up in 1-5 liters of LB media containing Turbo Amp™ (100 μg/μl) and chloramphenicol (30 μg/μl) at 30° C. with moderate aeration. The cells were collected by centrifugation and stored at −80° C. until use.

Cell pellets (12-24 grams) were resuspended in 3 volumes of lysis buffer (buffer A: 50 mM Tris HCl (pH 8.2), 1 mM EDTA, and 10 mM βME). Lysozyme (1 mg/g cells) and PMSF (1 mM) were added and the cells were lysed for 1 hour at 4° C. The cell mixture was sonicated, and the debris removed by centrifugation at 15,000 rpm for 30 minutes (4° C.). Tween 20 and Igepal CA-630 were added to final concentrations of 0.1% and the supernatant was heated at 72° C. for 10 minutes. Heat denatured E. coli proteins were then removed by centrifugation at 15,000 rpm for 30 minutes (4° C.).

The expression of JDF-3 and Pfu mutants was confirmed by SDS-PAGE (a band migrating at 95 kD).

### Example 3

### Evaluation of RT Activity by Radioactive Nucleotide Incorporation Assay

Partially-purified JDF-3 and Pfu mutant preparations (heat-treated bacterial extracts) were assayed to identify the most promising candidates for purification and comprehensive RT-PCR testing. To assess RT activity of the mutants, the relative RNA/DNA dependent DNA polymerization activity was measured for each mutant.

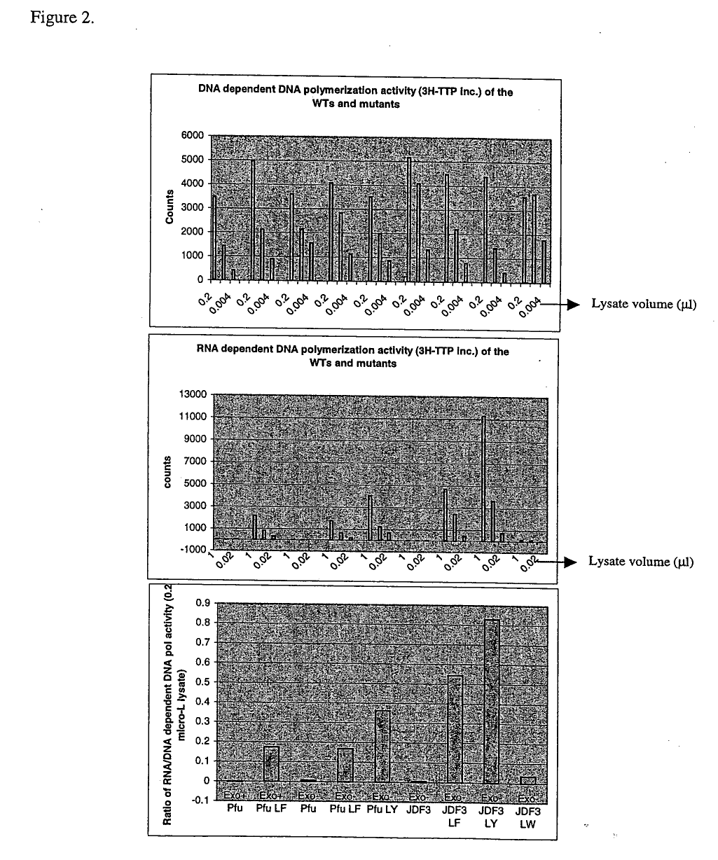

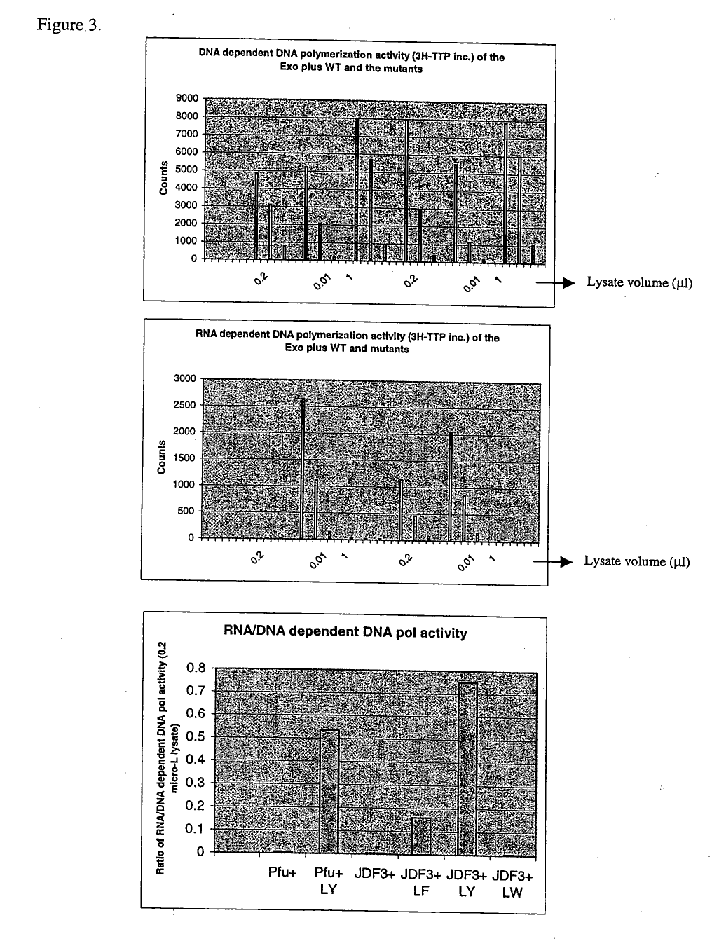

The DNA dependent DNA polymerization activity assay was performed according to a previously published method (Hogrefe, H. H., et al (01) Methods in Enzymology, 343:91-116). Relative dNTP incorporation was determined by measuring polymerase activity ([³H]-TTP incorporation into activated calf thymus DNA). A suitable DNA polymerase reaction cocktail contains: 1× cloned Pfu reaction buffer, 200 μM each dNTPs, 5 μM [³H]TTP (NEN #NET-221H, 1 mCi/ml, 20.5 Ci/mmole), 250 μg/ml of activated calf thymus DNA (Pharmacia #27-4575-01. Three different volumes of clarified lysates from WT and mutants (FIGS. 2 and 3) were used in a final reaction volume of 10 μl. Polymerization reactions were conducted in duplicate for 30 minutes at 72° C.

The extension reactions were quenched on ice, and 5 μl aliquots were spotted immediately onto DE81 ion-exchange filters (2.3 cm; Whatman #3658323). Unincorporated [³H]TTP was removed by 6 washes with 2×SSC (0.3M NaCl, 30 mM sodium citrate, pH 7.0), followed by a brief wash with 100% ethanol. Incorporated radioactivity was measured by scintillation counting. Reactions that lacked enzyme were set up along with sample incubations to determine “total cpms” (omit filter wash steps) and “minimum cpms”(wash filters as above). Sample cpms were subtracted by minimum cpms to determine “corrected cpms”.

The RNA dependent DNA polymerization assay was performed as follows. Relative dNTP incorporation was determined by measuring polymerase activity ([³H]-TTP incorporation into poly(dT):poly(rA) template (apbiotech 27-7878)). A suitable DNA polymerase reaction cocktail contains: 1× cloned Pfu reaction buffer, 800 μM TTP, 5 μM [³H]TTP (NEN #NET-601A, 65.8 Ci/mmole), 10 g poly(dT):poly(rA). Three different volumes of clarified lysates from WT and mutants (FIGS. 2 and 3) were used in a final reaction volume of 10 μl. Polymerization reactions were conducted in duplicate for 10 minutes at 50° C. followed by 30 minutes at 72° C.

The extension reactions were quenched on ice, and 5 μl aliquots were spotted immediately onto DE81 ion-exchange filters (2.3 cm; Whatman #3658323). Unincorporated [³H]TTP was removed by 6 washes with 2×SSC (0.3M NaCl, 30 mM sodium citrate, pH 7.0), followed by a brief wash with 100% ethanol. Incorporated radioactivity was measured by scintillation counting. Reactions that lacked enzyme were set up along with sample incubations to determine “total cpms” (omit filter wash steps) and “minimum cpms”(wash filters as above). Sample cpms were subtracted by minimum cpms to determine “corrected cpms”.

Partially purified preparations of the exo⁻ and exo⁺JDF-3 L408F and L408Y and Pfu L409F and L409Y showed improved RT activity compared to wild type JDF-3 and Pfu (FIGS. 2 and 3).

### Example 4

### Purification of JDF-3 and Pfu DNA Polymerase Mutants

JDF-3 and Pfu mutants can be purified as described in U.S. Pat. No. 5,489,523 (purification of the exo⁻ Pfu D141A/E143A DNA polymerase mutant) or as follows. Clarified, heat-treated bacterial extracts were chromatographed on a Q-Sepharose™ Fast Flow column (˜20 ml column), equilibrated in buffer B (buffer A plus 0.1% (v/v) Igepal CA-630, and 0.1% (v/v) Tween 20). Flow-through fractions were collected and then loaded directly onto a P11 Phosphocellulose column (˜20 ml), equilibrated in buffer C (same as buffer B, except pH 7.5). The column was washed and then eluted with a 0-0.7M KCl gradient/Buffer C. Fractions containing DNA polymerase mutants (95 kD by SDS-PAGE) were dialyzed overnight against buffer D (50 mM Tris HCl (pH 7.5), 5 mM βME, 5% (v/v) glycerol, 0.2% (v/v) Igepal CA-630, 0.2% (v/v) Tween 20, and 0.5M NaCl) and then applied to a Hydroxyapatite column (˜5 ml), equilibrated in buffer D. The column was washed and DNA polymerase mutants were eluted with buffer D2 containing 400 mM KPO4, (pH 7.5), 5 mM βME, 5% (v/v) glycerol, 0.2% (v/v) Igepal CA-630, 0.2% (v/v) Tween 20, and 0.5 M NaCl. Purified proteins were spin concentrated using Centricon YM30 devices, and exchanged into final dialysis buffer (50 mM Tris-HCl (pH 8.2), 0.1 mM EDTA, 1 mM dithiothreitol (DTT), 50% (v/v) glycerol, 0.1% (v/v) Igepal CA-630, and 0.1% (v/v) Tween 20).

Protein samples were evaluated for size, purity, and approximate concentration by SDS-PAGE using Tris-Glycine 4-20% acrylamide gradient gels. Gels were stained with silver stain or Sypro Orange (Molecular Probes). Protein concentration was determined relative to a BSA standard (Pierce) using the BCA assay (Pierce).

Mutant proteins were purified to ˜90% purity as determined by SDS-PAGE.

### Example 5

### Evaluation of RT Activity of Purified Mutants by Radioactive Nucleotide Incorporation Assay

The RNA dependent DNA polymerization assay was performed as follows. Relative dNTP incorporation was determined by measuring polymerase activity ([³³P]-dGTP incorporation into poly(dG):poly(rC) template (apbiotech 27-7944)). A suitable DNA polymerase reaction cocktail contains: 1× cloned Pfu reaction buffer, 800 μM dGTP, 1 μCi [³³P]dGTP (NEN #NEG-614H, 3000 Ci/mmole), 10 μg poly(dG):poly(rC). The final reaction volume was 10 μl. Polymerization reactions were conducted in duplicate for 10 minutes at 50° C. followed by 30 minutes at 72° C.

The extension reactions were quenched on ice, and 511 aliquots were spotted immediately onto DE81 ion-exchange filters (2.3 cm; Whatman #3658323). Unincorporated [³³P]dGTP was removed by 6 washes with 2×SSC (0.3M NaCl, 30 mM sodium citrate, pH 7.0), followed by a brief wash with 100% ethanol. Incorporated radioactivity was measured by scintillation counting. Reactions that lacked enzyme were set up along with sample incubations to determine “total cpms” (omit filter wash steps) and “minimum cpms”(wash filters as above). Sample cpms were subtracted by minimum cpms to determine “corrected cpms”.

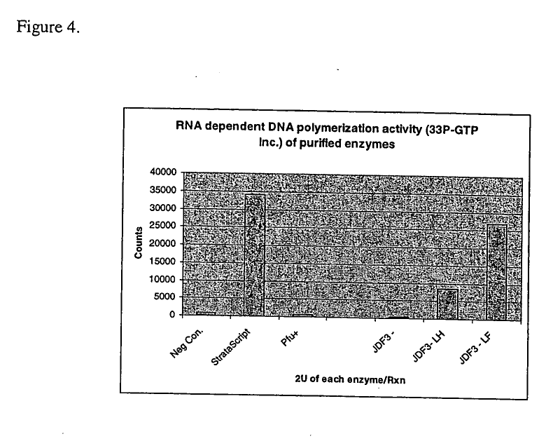

Purified preparations of the exo⁻ JDF-3 L408H and L408F showed improved RT activity compared to wild type JDF-3 and Pfu (FIG. 4). RT activity of 2 units of StrataScript (Stratagene's RNase H minus MMLV-RT) was determined in the same assay for comparison.

### Example 6

### Evaluation of RT Activity of Purified Mutants by RT-PCR Assay

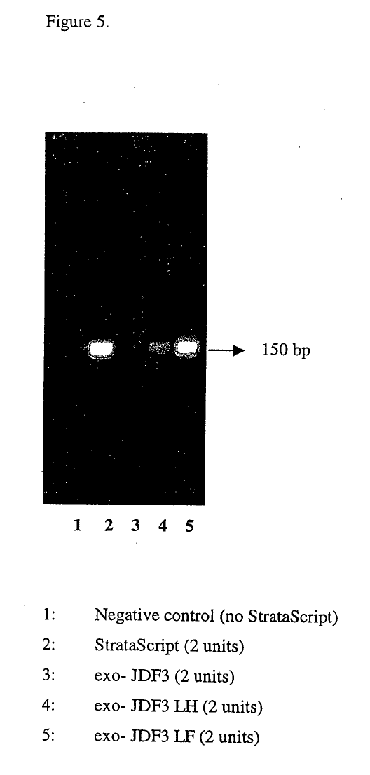

Each RT assay was carried out in a total reaction volume of 10 μl. The final reagent concentrations were as follows: 18 pmol oligo(dT)₁₈, 1 mM each dNTPs, 500 ng human total RNA in either 1× StrataScript buffer (Stratagene) for StrataScript or 1× cloned Pfu buffer (Stratagene) for Pfu, JDF3 WT and mutants. StrataScript reactions were incubated at 42° C. for 40 minutes. WT Pfu, JDF3 and the mutants were incubated at 50° C. for 5 minutes followed by 72° C. for 30 minutes. 2 μl of each cDNA synthesis reaction was used in a PCR containing 2.5 units Taq DNA polymerase, 200 μM each dNTP, 100 ng of each of GAPDH-F and GAPDH-R primers (FIG. 1) in 1× Taq 2000 buffer (Stratagene). Amplification reactions were carried out using the temperature cycling profile as follows: 35 cycles of 95° C. for 30 s, 55° C. for 30 s, and 720 for 1 min. 5 μl of each PCR was run on a 1% agarose gel and stained with ethidium bromide (FIG. 5).

Since the DNA amplification portion of each reaction was performed with the same enzyme (Taq), these results demonstrated that exo⁻ JDF3 L408F exhibit higher reverse transcription efficiency than exo⁻ JDF3 L408H (FIG. 5). The RT activity of the exo⁻ JDF3 is similar to the negative control (no StrataScript).

### Example 7

### Evaluation of DMSO Effect on RT Activity of Purified exo+ Pfu L409Y

In order to evaluate the effect of DMSO concentration on RT activity of mutant Family B DNA polymerase, a cDNA synthesis reaction was carried out using exo+ Pfu L409Y DNA polymerase in the presence of varying amounts of DMSO. Reactions were carried out in a total volume of 20 μl. The final reagent concentrations were as follows: 1000 ng of exo+ Pfu L409Y, 90 pmol oligo(dt)₁₈, 0.8 mM each dNTPs, 3 μg RNA size marker (Ambion, cat. 7150) in 1× StrataScript buffer (Stratagene). A range of 0-25% DMSO was added to the reactions. Reactions were incubated at 50° C. for 3 minutes followed by 65° C. for 60 minutes. The entire volume of each reaction was run on a 1% alkaline agarose gel and stained with ethidium bromide.

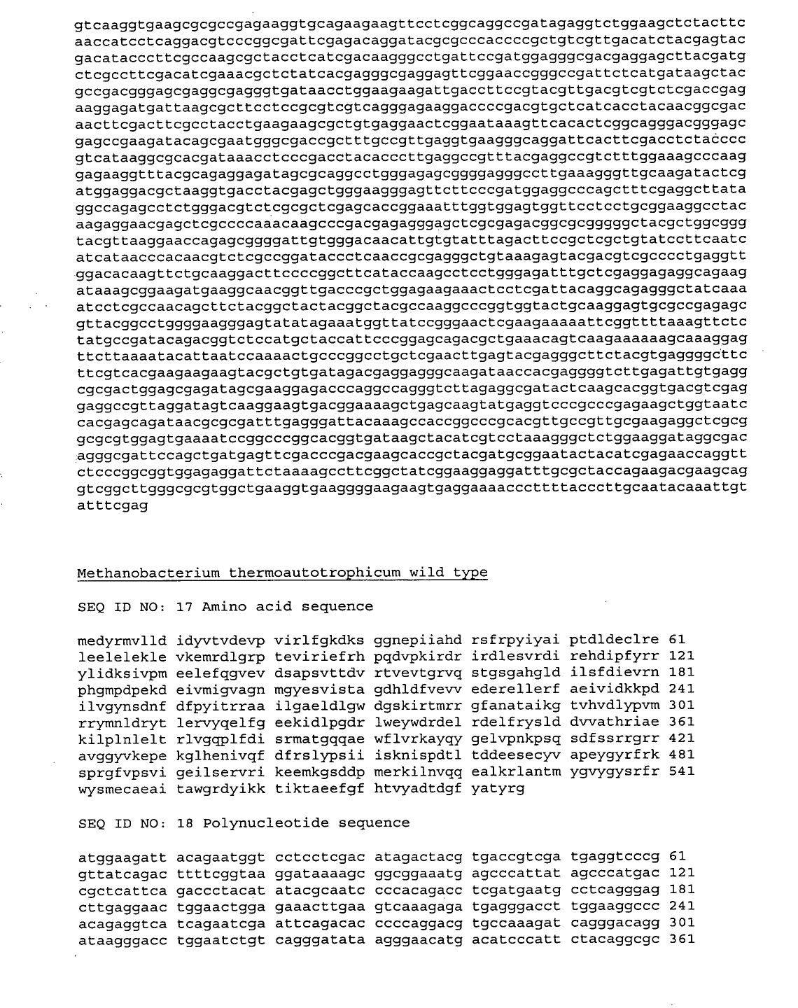

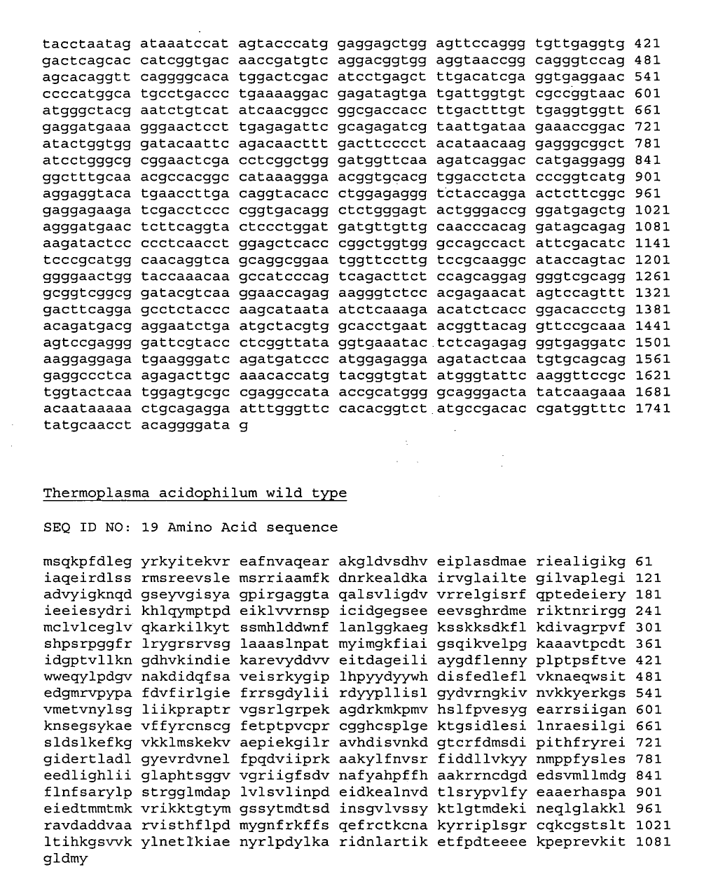

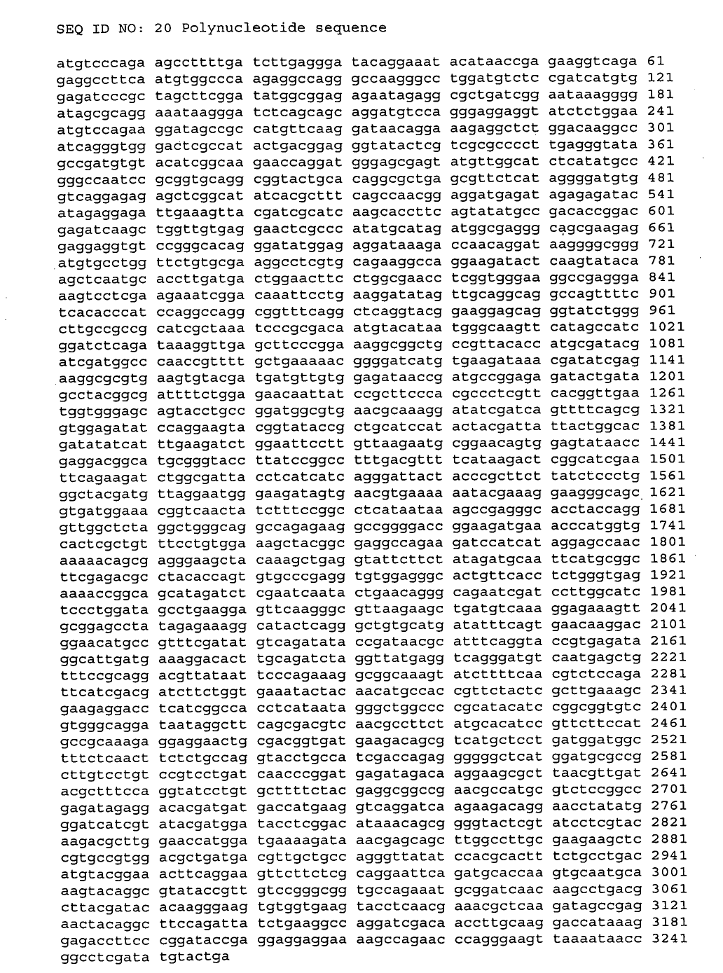

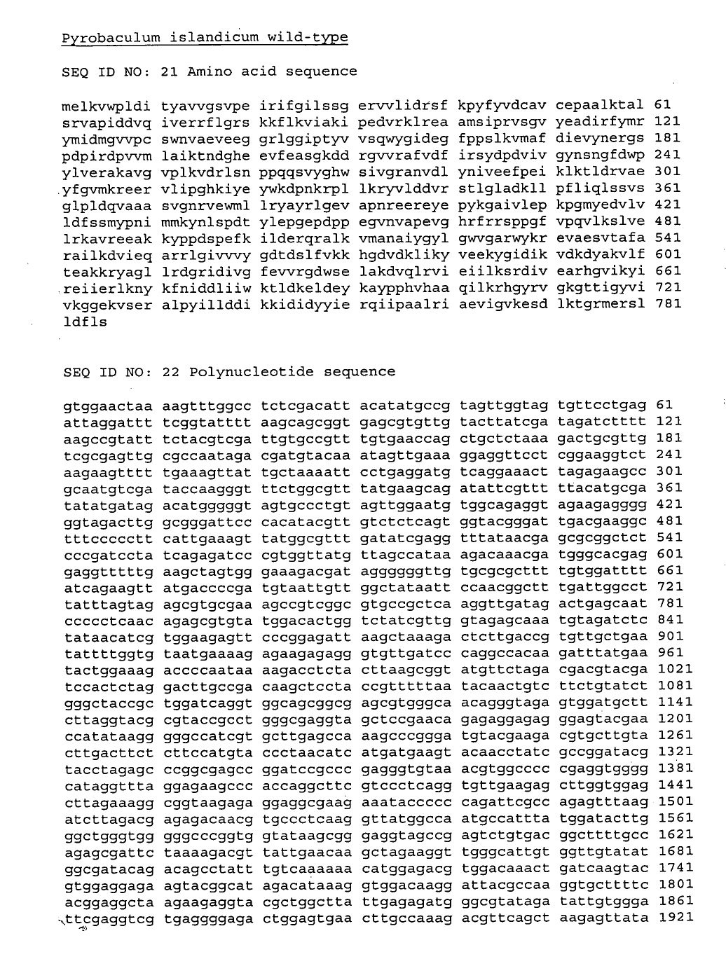

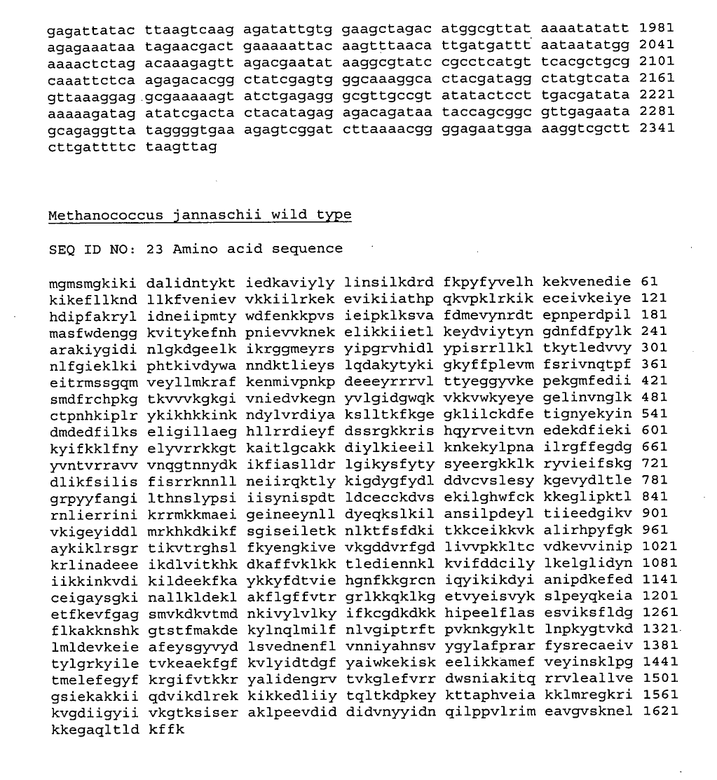

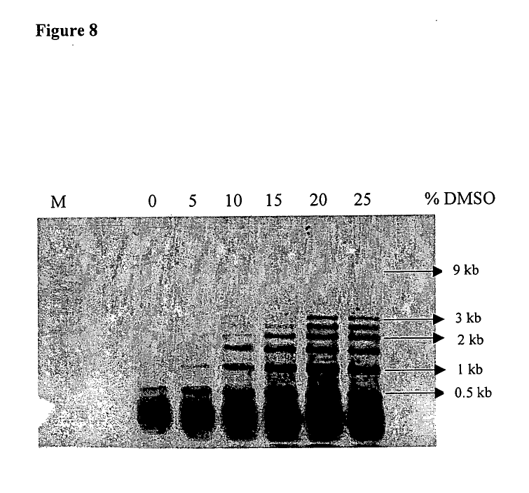

The results shown in FIG. 8 demonstrate that adding DMSO significantly improves the reverse transcriptase activity of exo+ Pfu L409Y.

### Example 8

### Mutant Pfu L409Y Amino Modified Nucleotide Incorporation

In order to evaluate the efficiency of amino modified nucleotide incorporation by mutant Pfu, cDNA synthesis reactions were conducted with Pfu L409Y DNA polymerase.

Five cDNA synthesis reactions were performed (four reactions with Pfu L409Y and one reaction with STRATASCRIPT reverse transcriptase. The first reaction contained unmodified dNTPs. Reaction two contained a two fold excess of amino allyl modified dUTP over dTTP. Reaction three contained a two-fold excess of amino allyl dCTP over dCTP. Reaction four contained a two-fold excess of amino allyl dUTP over dTTP and a two-fold excess of amino allyl dCTP over dCTP. Reaction five utilized the FAIRPLAY Microarray Labeling Kit (Stratagene, La Jolla, Calif.; Cat# 252002), containing amino allyl dUTP and STRATASCRIPT reverse transcriptase (Stratagene, La Jolla, Calif.; catalog #252002).

With the exception of the 20X-dNTP solution all reaction components used in the reverse transcriptase reactions using Pfu L409Y DNA polymerase were obtained from the FAIRPLAY Microarray Labeling Kit (Stratagene, La Jolla, Calif.; Cat# 252002). Reactions conducted with STRATASCRIPT reverse transcriptase, (Stratagene, La Jolla, Calif.: Cat.# 60085) used all reaction components from the FAIRPLAY Microarray Labeling Kit (Stratagene, La Jolla, Calif.; Cat# 252002) according to manufacturers instructions.

Incorporation of aa-dUTP and/or aa-dCTP into cDNA was as follows: 3 ul of 1 ug/ul RNA ladder (Millenium RNA ladder, Ambion) was combined with 1 ul of 0.5 ug/ul oligo dT primer (18 mer; TriLink) in a total volume of 8 ul. The RNA and oligo dT primer were annealed by heating the sample at 70° C. for 10 minutes (min) and then cooled on ice. To prepare modified cDNA, 2 ul of 10× STRATASCRIPT buffer, 1.5 ul of 0.1 mM dithiothreitol (DTT), 1 ul of 20× dNTP mixture (20× is 16 mM dGTP, 16 mM dCTP, 16 mM dATP, 16 mM dTTP and aa dUTP (Trilink) or 16 mM dGTP, 16 mM dTTP, 16 mM dATP, 16 mM dCTP and aa dCTP (Trilink)), 4 ul of 100% (v/v) dimethylsulfoxide (DMSO), 0.5 ul of (40 units/ul) RNase block, and RNase-free H₂O to a total reaction volume of 19 ul were combined and added to the annealed RNA and oligo dT. The reaction was mixed well and 1 ul of 1 ug/ul Pfu RT (Exo+, L409Y) was added. The reaction was incubated at 45° C. for 5 minutes and then 65° C. for 1-2 hour(s). One fourth of each reaction containing the amino-modified cDNA containing the non-conventional nucleotide, amino allyl-dUTP or amino allyl-dCTP, was then analyzed by denaturing alkaline agarose gel electrophoresis to determine the relative cDNA yield and length, respectively.

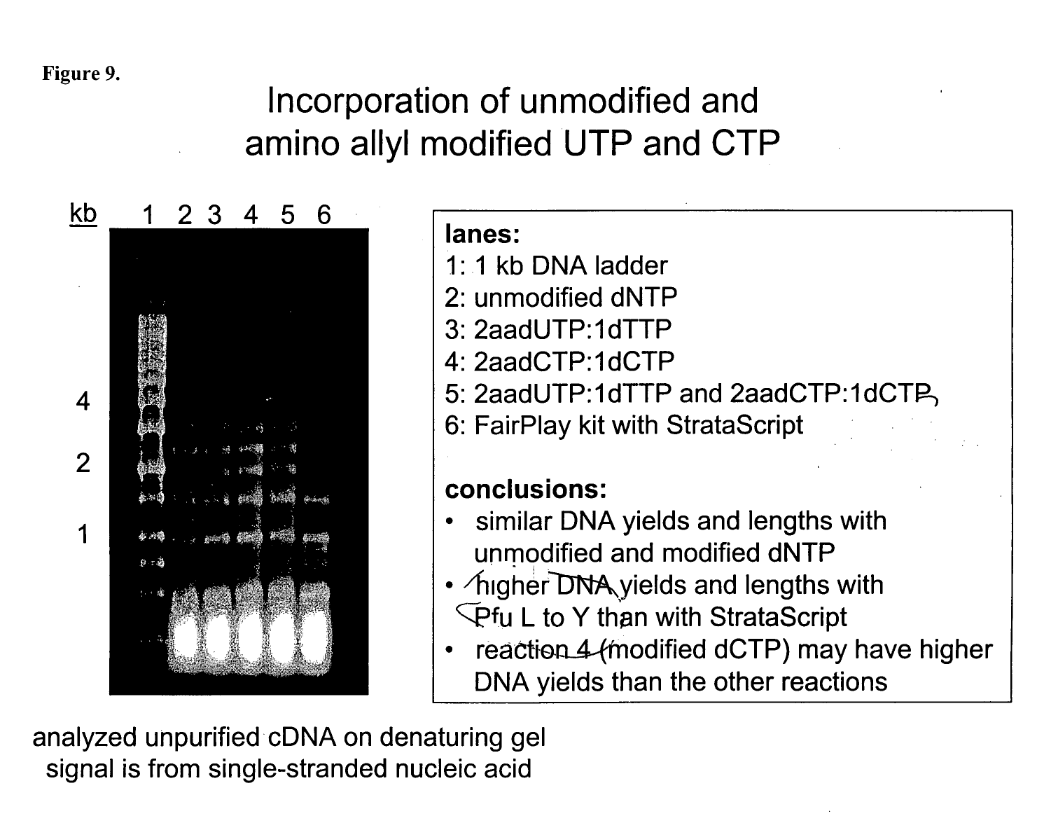

The results shown in FIG. 9 demonstrate that Pfu L409Y generates comparable DNA yields and lengths with unmodified and amino allyl modified dNTPs. Furthermore, Pfu L409Y generates higher yields and lengths than STRATASCRIPT reverse transcriptase (Stratagene, La Jolla, Calif.; catalog #252002).

### Example 9

### Mutant Pfu L409Y Amino Allyl Modified DNA Coupled to Cy5

To confirm the ability of Pfu L409Y to generate high yields and lengths of amino allyl modified DNA, a second reaction was conducted to generate amino allyl modified DNA coupled to Cy5. The remaining reaction volumes from Example 8 were hydrolyzed to remove RNA by adding 10 ul of 1 N NaOH and incubating at 70° C. for 10 min. To neutralize the reaction, the reaction was cooled to room temperature, spun to collect the condensate, and 10 ul of HCl was added. To precipitate the purified cDNA, 4 ul 3 M sodium acetate, pH 5.2, 1 ul of 20 ug/ul glycogen (Roche), and 100 ul of ice-cold 100% ethanol were added and incubated at −20° C. for a minimum of 30 min. The samples were spun at 14,000×g for 15 min at 4° C. and the supernatants decanted. The pellets were washed with 0.5 ml 70% ethanol, respun and the supernatants decanted and the cDNA pellets were air-dried.

The amino-modified cDNA was coupled to the amine-reactive fluorescent dye as follows. The cDNA pellet from one reaction was resuspended in 4.5 ul of 0.1 M sodium bicarbonate buffer, pH 9.0, combined with 12.5-18.8 ng monofunctional NHS-ester Cy3 or Cy5 dye (Amersham Pharmacia Biotech) in 10 ul DMSO and incubated in the dark at room temperature for 1 hour. The fluorescence-labeled cDNA was purified and concentrated to ˜15 ul using the purification columns from the FAIRPLAY Microarray Labeling Kit (Stratagene, La Jolla, Calif.; Cat# 252002) according to manufacturers instructions.

The fluorescence-coupled cDNA was analyzed by agarose gel electrophoresis analysis. A thin agarose gel was prepared by pouring 2% (w/v) agarose gel in 1×TAE buffer on a 2 cm×3 cm glass microscope slide. One fourth of the labeled cDNA from each reaction was loaded onto the gel and electrophoresed at 125 volts (V) for 0.5 hour. The Cy-5 labeled cDNA was visualized using a 2 color, laser/PMT Prototype Microarray Scanner (John Parker; UCLA). Cy5 was detected with a 635 nm laser with 700 nm-emission filter.

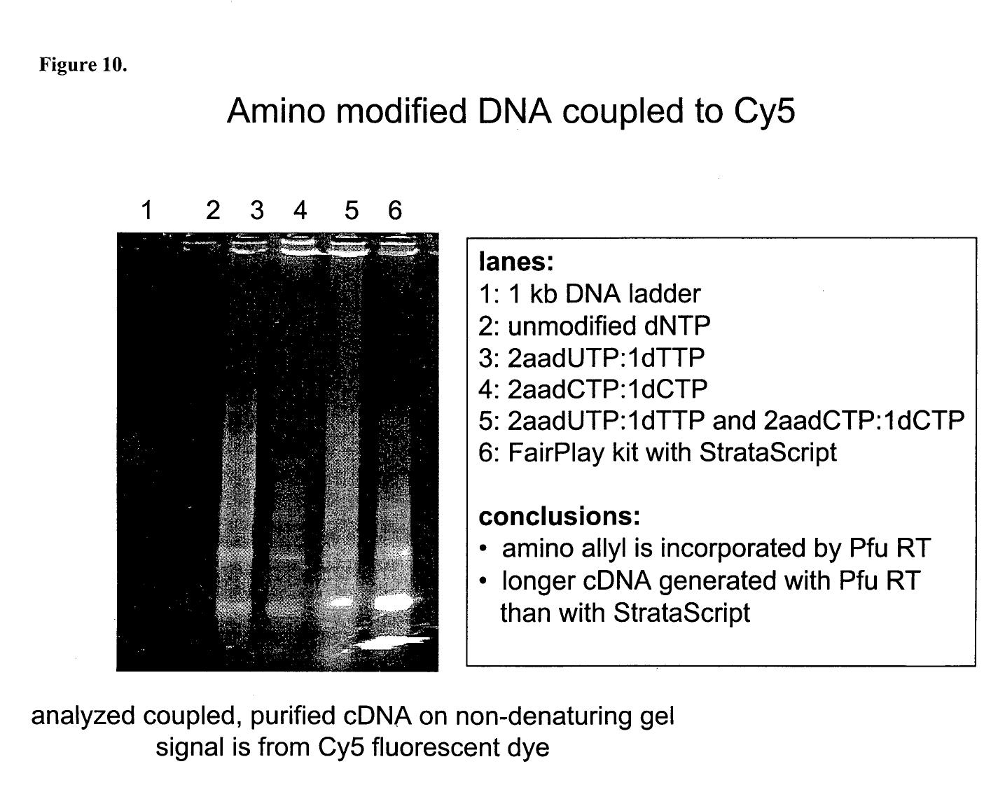

The results shown in FIG. 10, confirm demonstrate that Pfu L409Y generates comparable DNA yields and lengths with unmodified and amino allyl modified dNTPs when compared with STRATASCRIPT reverse transcriptase (Stratagene, La Jolla, Calif.; catalog #252002).

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

### REFERENCES

1. Joyce, C. M., Kelley, W. S. and Grindley N. D. F. (1982) J. Biol. Chem. 257, 1958-1964.
2. Lopes, P. Martinez, S., Diaz, A. Espinosa, M. And Lacks, S. A. (1989) J. Biol. Chem. 264, 4255-4263.
3. Lawyer, F. C., Stoffel, S., Saiki, R. K., Myambo, K. Drummond, R. and Gelfand, D. H. (1989) J. Biol. Chem. 264, 6427-6437.
4. Akhmetzjanov, A. A. and Vakhitov, V. A. (1992) Nucl. Acids Res. 20, 5839.
5. Leavitt, M. C. and Ito, J. (1989) Proc. Acad. Sci. U.S.A. 86, 4465-4469.
6. Dunn, J. J. and Studier, F. W. (1983) J. Mol. Biol. 166, 477-535.
7. Scarlato, V. And Gargano, S. (1992) Gene 118, 109-113.
8. Ràdén, B. And Rutberg, L. (1984) J. Virol. 52, 9-15.
9. Foury, F. (1989) J. Biol. Chem. 264, 20552-20560.
10. Ito, J. And Braithwaite, D. K. (1990) Nucl. Acids Res. 18, 6716.
11. Blanco, L. Bernad, A. And Salas, M. (1991) Nucl. Acids res. 19,955.
12. Hahn, S. And Rüger, W, (1989) Nucl. Acids Res. 17, 6729.
13. Hollingsworth, H. C. and Nossal, N. G. (1991) J. Biol. Chem. 266, 1888-1897.
14. Kaliman, A. V., Krutilina, A. I., Kryukov, V, M. and Bayev, A. A. (1986) FEBS Lett. 195, 61-64.
15. Iwasaki, H. Ishino, Y., Toh, H. Nakata, A. and Shinagawa, H. (1991) Mol. Gen Genet. 226, 24-33.
16. Jung, G., Leavitt, M. C., Hsieh, J.-C. and Ito, J. (1987) Proc. Natl. Acad. Sci. U.S.A. 84, 8287-8291.
17. Savilahti, H. And Bamford D. H. (1987) Gene 57, 121-130.
18. Yoshikawa, H. And Ito, J. (1982) Gene 17, 323-335.
19. Matsumoto, K., Takano, H., Kim, C. I. and Hirokawa, H. (1989) Gene 84, 247-255.
20. Spicer, E. K., Rush, J. Fung, C., Reha-Krantz, L. J., Karam, J. D. and Konigsberg, W. H. (1988) J. Biol. Chem. 263, 7478-7486.
21. Perler, F. B., Comb, D. G., Jack, W. E., Moran, L. S., Qiang, B., Kucera, R. B., Benner, J., Slatko, B. E., Nwankwo, D. O., Hempstead, S. K., Carlow, C. K. S. and Jannasch, H. (1992) Proc. Natl. Acad. Sci. USA 89, 5577-5581.
22. Mathur, E. J., Adams, M. W., Callen, W. N. and Cline, J. M. (1991) Nucleic. Acids Res. 19, 6952.
23. Pisani, F. W., De Martino, C. and Rossi, M. (1992) Nucl. Acids Res. 20, 2711-2716.
24. Wong S., W. Wahl, A. F., Yuan, P.-M., Arai, N., Pearson, B. E., Arai, K, -i., Korn, D., Hunkapiller, M. W. and Wang, T. S.-F. (1988) EMBO J. 7, 37-47.
25. Pizzagalli, A., Valsasnini, P., Plevani, P. and Lucchini, G. (1988) Porc. Natl. Acad. Sci. U.S.A. 85, 3772-3776.
26. Damagnez, V., Tillit, J., deRecondo, A.-M. and Baldacci, G. (1991) Mol. Gen. Genet. 226, 182-189.
27. Hirose, F., Yamaguchi, M. Nishida, Y., Masutani, M., Miyazawa, H., Hanaoka, F. and Matsukage, A. (1991) Nucl. Acids Res. 19, 4991-4998.
28. Leegwater, P. A. J., Strating, M., Murphy, N. B., Kooy, R. F., van der Vliet, P. C. and Overdulve, J. P. (1991) Nucl. Acids Res. 19, 6441-6447.
29. Chung, D. W., Zhang, J., Tan C.-K., Davie, E. W., So, A. G. and Downey, K. M. (1991) Proc. Natl. Acad. Sci. USA 88, 11197-11201.
30. Yang, C.-L., Chang, L. S., Zhang, P., Hao, H., Zhu, L., Tommey, N. L. and Lee, M. Y. W. T. ((1992) Nucl. Acids Res. 20, 735-745.
31. Zhang, J. Chung, D. W., Tan, C.-K., Downey, K. M., Davie, E. W. and So, A. G. (1991) Biochemistry 30, 11742-11750.
32. Morrison, A. and Sugino, A. (1992) Nucl. Acids Res. 20, 375.
33. Pignéde, G., Bouvier, D., deRecondo, A.-M. And Baldacci, G. (1991) J. Mol. Biol. 222, 209-218.
34. Ridley, R. G., White, J. H., McAleese, S. M., Gorman, M., Alano, P., deVies, E. and Kilbey, B. J. (1991) Nucl. Acids Res. 19, 6731-6736.
35. Morrison, A., Araki, H., Clark, A. B., Hamatake, R. K. and Sugino, A. (1990) Cell 62, 1143-1151.
36. Morrison, A., Christensen, R. B., Alley, J., Beck, A. K., Bemstine, E. G., Lemontt, J. F. and Lawrence, C. W. (1989) J. Bacteriol. 171, 5659-5667.
37. Gibbs, J. S., Chiou, H. C., Hall, J. D., Mount, D. W., Retondo, M. J., Weller, S. K. and Coen, D. M. (1985) Proc. Natl. Acad. Sci. U.S.A. 82, 7969-7973.
38. Telford, E. A., Watson, M. S., McBride, K. and Davison, A. J. (1992) Virology 189, 304-316.
39. Davison, A. J. and Scott, J. E. (1986) J. Gen. Virol. 67, 1759-1816.
40. Baer, R., Bankier, A. T. Biggin, M. D., Deininger, P. L., Farrell, P. J., Gibson, T. J., Hatfull, G., Hudson, G. S., Satchwell, S. C., Séguin, C., Tuffnell, P. S. and Barrell, B. G. (1984) Nature 310,207-211.
41. Albrecht, J.-C. and Fleckenstein, B. (1990) Virology 174, 533-542.
42. Kouzarides, T. Bankier, A. T., Satchwell, S. C., Weston, K., Tomlinson, P. and Barrell, B. G. (1987) J. Virol, 61, 125-133.
43. Elliott, R., Clark, C. Jaquish, D. and Spector, D. H. (1991) Virology n185, 169-186.
44. Teo, I. A., Griffin, B. E. and Jones, M. D. (1991) J. Virol. 65, 4670-4680.
45. Davison, A. J. (1992) Virology 186, 9-14.
46. Grabherr, R., Strasser, P. and Van Etten, J. L. (1992) Virology 188, 721-731.
47. Binns, M. M., Stenzler, L. Tomley, F. M., Campbell, J. and Broursnell, M. E. G. (1987) Nucl. Acids Res. 15, 6563-6573.
48. Earl P. L., Jones, E. V. and Moss, B. (1986) Prov. Natl. Acad. Sci. U.S.A. 83, 3659-3663.
49. Mustafa, A. And Yuen, L. (1991) DNA Seq. 2, 39-45.
50. Tomalski, M. D., Wu, J. and Miller, L. K. (1988) Virology 167, 591-600.
51. Bjornson, R. M. and Rohrmann, G. F. (1992) J. Gen. Virol 73, 1499-1504.
52. Gingeras, T. R., Sciaky, D., Gelinas, R. E., Bing-Dong, J., Yen, C. E., Kelly, M. M., Bullock, P. A. Parsons, B. L., O'Neill. K. E. and Roberts, R. J. (1982) J. Biol. Chem, 257, 13475-13491.
53. Engler, J. A., Hoppe, M. S. and van Bree, M. P. (1983) Gene 21, 145-159.
54. Shu, L., Hing, J. S., Wei, Y.-f. and Engler, J. A., (1986) Gene 46, 187-195.
55. Paillard, M., Sederoff, R. R. and Levings, C. S. III (1985) EMBRO J. 4, 1125-1128.
56. Chan, B. S.-S., Court, D. A., Vierula, P. J. and Bertrand, H. (1991) Curr. Genet. 20, 225-237.
57. Kempken, F., Meinhardt, F. and Esser, K. (1989) Mol. Gen. Genet, 218, 623-530.
58. Oester, B. And Tudzynski, P. (1989) Mol. Gen. Genet. 217, 132-140.
59. Court D. A. and Bertrand, H. (1992) Curr. Genet. 22, 385-397.
60. Robison, M. M., Royer, J. C. and Horgen, P. A. (1991) Curr. Genet. 19, 495-502.
61. Stark, M. J. R., Mileham, A. J., Romanos, M. A. and Boyd, A. (1994) Nucl. Acids Res. 12, 6011-6030.
62. Tommasino, M. Ricci, S. and Galeotti, C. L. (1988) Nucl. Acids Res. 16, 5863-5878.
63. Hishinuma, F. and Hirai, K. (1991) J. Gen. Genet. 226, 97-106.
64. Hopfiier, K. P. et al. (1999) Proc. Natl. Acad. Sci. U.S.A. 96, 3600-3605.
65. Niehaus, F. et al. (1997) Gene 204, 153-158.
66. Tagaki et al. (1997) Appl. Environ. Microbiol. 63, 4504-4510.
67. Datukishvili, N. et al. (1996) Gene 177, 271-273.
68. Southworth, M. W. et al. (1996) Proc. Natl. Acad. Sci. U.S.A. 93, 5281-5285.
69. Uemori, T. et al. (1995) J. Bacteriol. 177, 2164-2177.
70. Konisky, J. et al. (1994) J. Bacteriol. 176, 6402-6403.
71. Zhao (1999) Structure Fold Des. 7, 1189.
72. Lai, E., Riley, J., Purvis, I. & Roses, A. A 4-Mb high-density single nucleotide polymorphism-based map around human APOE. Genomics 54, 31-8. (1998).
73. Saiki, R. K., Walsh, P. S., Levenson, C. H. & Erlich, H. A. Genetic analysis of amplified DNA with immobilized sequence-specific oligonucleotide probes. Proc Natl Acad Sci USA 86, 6230-4. (1989).
74. Landegren, U., Kaiser, R., Caskey, C. T. & Hood, L. DNA diagnostics—molecular techniques and automation. Science 242, 229-37. (1988).
75. Shi, M. M. Enabling large-scale pharmacogenetic studies by high-throughput mutation detection and genotyping technologies. Clin Chem 47, 164-72. (2001).
76. Livak, K. J., Marmaro, J. & Todd, J. A. Towards fully automated genome-wide polymorphism screening. Nat Genet 9, 341-2. (1995).
77. Tyagi, S., Bratu, D. P. & Kramer, F. R. Multicolor molecular beacons for allele discrimination. Nat Biotechnol 16, 49-53. (1998).
78. Gilles, P. N., Wu, D. J., Foster, C. B., Dillon, P. J. & Chanock, S. J. Single nucleotide polymorphic discrimination by an electronic dot blot assay on semiconductor microchips. Nat Biotechnol 17, 365-70. (1999).
79. Fu, D. J. et al. Sequencing exons 5 to 8 of the p53 gene by MALDI-TOF mass spectrometry. Nat Biotechnol 16, 381-4. (1998).
80. Chen, X. & Kwok, P. Y. Template-directed dye-terminator incorporation (TDI) assay: a homogeneous DNA diagnostic method based on fluorescence resonance energy transfer. Nucleic Acids Res 25, 347-53. (1997).
81. Syvanen, A. C., Aalto-Setala, K., Harju, L., Kontula, K. & Soderlund, H. A primer-guided nucleotide incorporation assay in the genotyping of apolipoprotein E. Genomics 8, 684-92. (1990).
82. Taylor, J. D. et al. Flow cytometric platform for high-throughput single nucleotide polymorphism analysis. Biotechniques 30, 661-6, 668-9. (2001).
83. Chen, X., Zehnbauer, B., Gnirke, A. & Kwok, P. Y. Fluorescence energy transfer detection as a homogeneous DNA diagnostic method. Proc Natl Acad Sci USA 94, 10756-61. (1997).
84. Tabor, S. & Richardson, C. C. A single residue in DNA polymerases of the Escherichia coli DNA polymerase I family is critical for distinguishing between deoxy- and dideoxyribonucleotides. Proc Natl Acad Sci USA 92, 6339-43. (1995).
85. Gardner, A. F. & Jack, W. E. Determinants of nucleotide sugar recognition in an archaeon DNA polymerase. Nucleic Acids Res 27, 2545-53. (1999).
86. Evans, S. J. et al. Improving dideoxynucleotide-triphosphate utilisation by the hyper-thermophilic DNA polymerase from the archaeon Pyrococcus furiosus. Nucleic Acids Res 28, 1059-66. (2000).
87. Kong et al., J. Biol. Chem. 268:1965 (1993)
88. U.S. Pat. No. 5,210,036
89. U.S. Pat. No. 5,322,785
90. Xu et al., Cell 75 (7), 1371-1377 (1993)
91. Lundberg et al., Gene 108:1 (1991)
92. PCT Pub. WO 92/09689
93. U.S. Pat. No. 5,948,663
94. U.S. Pat. No. 5,866,395
95. U.S. Pat. No. 6,008,025
96. U.S. Pat. Nos. 5,602,011; 5,948,663; 5,866,395; 5,545,552; 5,556,772
97. Salhi et al., J. Mol. Biol., 209:635-641 (1989).
98. Salhi et al., Biochem. Biophys. Res. Comm., 167:1341-1347 (1990).
99. Rella et al., Ital. J. Biochem., 39:83-99 (1990).
100. Forterre et al., Can. J. Microbiol., 35:228-233 (1989).
101. Rossi et al., System. Appl. Microbiol., 7:337-341 (1986).
102. Klimczak et al., Nucleic Acids Res., 13:5269-5282 (1985).
103. Elie et al., Biochim. Biophys. Acta 951:261-267 (1988).

1. A composition comprising a mutant Family B DNA polymerase and at least one amino allyl modified nucleotide, wherein the mutant Family B DNA polymerase exhibits an increased reverse transcriptase activity.

2. A kit comprising a mutant Family B DNA polymerase, at least one amino allyl modified nucleotide, and packaging materials therefor, wherein the mutant Family B DNA polymerase exhibits an increased reverse transcriptase activity.

3. A method of generating a modified complementary strand of DNA, the method comprising:
combining a template DNA molecule with a mutant Family B DNA polymerase, exhibiting an increased reverse transcriptase activity, in a reaction mixture comprising at least one non-conventional nucleotide, under conditions and for a time sufficient to permit the mutant Family B DNA polymerase to synthesize a complementary DNA strand incorporating the non-conventional nucleotide into the synthesized complementary DNA strand.

4. The method of claim 3, wherein the mutant Family B DNA polymerase is the mutant of a wild-type Family B DNA polymerase that has an LYP motif in Region II at a position corresponding to L409 of Pfu DNA polymerase.

5. The method of claim 3, wherein the mutant Family B DNA polymerase is a mutant of a wild type polymerase selected from the group consisting of Pfu DNA polymerase and JDF-3 DNA polymerase.

6. The method of claim 3, wherein the mutant Family B DNA polymerase is a mutant of a wild-type Family B DNA polymerase comprising an amino acid sequence selected from the group consisting of SEQ ID Nos. 1, 3, 5, 7, 9, 11, 13, 15, 17, 19, 21 and 23.

7. The method of claim 3, 4, 5 or 6, wherein the mutant Family B DNA polymerase comprises an amino acid mutation at the amino acids corresponding to L409 to P411 of SEQ ID NO:3

8. The method of claim 3, 4, 5 or 6, wherein said mutant Family B DNA polymerase further exhibits a decreased 3′-5′ exonuclease activity.

9. The method of claim 3, 4, 5 or 6, wherein the mutant Family B DNA polymerase further exhibits a reduced base analog detection activity.

10. The method of claim 3, 4, 5 or 6, wherein the mutant Family B DNA polymerase further exhibits a decreased 3′-5′ exonuclease activity and a reduced base analog detection activity.

11. The method of claim 3, 4, 5 or 6, wherein the mutant Family B DNA polymerase comprises an amino acid mutation at the position corresponding to L409 of SEQ ID NO: 3.

12. The method of claim 11, wherein the amino acid mutation at the amino acid corresponding to L409 of SEQ ID NO: 3 is a leucine to phenylalanine mutation, leucine to tyrosine mutation, leucine to histidine mutation or a leucine to tryptophan mutation.

13. The method of claim 3 wherein the non-conventional nucleotide is selected from the group consisting of: dideoxynucleotides, ribonucleotides, amino allyl modified nucleotides and conjugated nucleotides.

14. The method of claim 13, wherein the conjugated nucleotides are selected from the group consisting of radiolabeled nucleotides, fluorescently labeled nucleotides, biotin labeled nucleotides, chemiluminescently labeled nucleotides and quantum dot labeled nucleotides.

15. The method of claim 13, further comprising a coupling step.

16. The method of claim 15, wherein the coupling step comprising coupling the modified cDNA to a fluorescent dye containing a NHS- or STP-ester leaving group.

17. A method for amplifying an RNA molecule, comprising:
incubating a template RNA molecule with a primer complex in a first reaction mixture comprising a mutant Family B DNA polymerase exhibiting an increased reverse transcriptase activity, wherein the incubation permits synthesis of a complementary DNA template and wherein the primer complex comprises a primer complementary to the target sequence and a promoter region;
incubating the complementary DNA template in a second reaction mixture wherein the second reaction mixture permits synthesis of a second complementary DNA containing the promoter region; and
transcribing copies of RNA initiated from the promoter region of the primer complex, wherein the transcription generates anti-sense RNA.

18. The method of claim 17, wherein the mutant Family B DNA polymerase is a mutant of a wild-type Family B DNA polymerase that has an LYP motif in Region II at a position corresponding to L409 of Pfu DNA polymerase.

19. The method of claim 17, wherein the mutant Family B DNA polymerase is a mutant of a wild type polymerase selected from the group consisting of: Pfu DNA polymerase and a JDF-3 DNA polymerase.

20. The method of claim 17, wherein the mutant Family B DNA polymerase is a mutant of a wild-type Family B DNA polymerase comprising an amino acid sequence selected from the group consisting of SEQ ID Nos. 1, 3, 5, 7, 9, 11, 13, 15, 17, 19, 21 and 23.

21. The method of claim 17, 18, 19 or 20, wherein the mutant Family B DNA polymerase further exhibits a decreased 3′-5′ exonuclease activity.

22. The method of claim 17, 18, 19 or 20, wherein the mutant Family B DNA polymerase further exhibits a reduced base analog detection activity.

23. The method of claim 17, 18, 19 or 20 wherein the mutant Family B DNA polymerase further exhibits a decreased 3′-5′ exonuclease activity and a reduced base analog detection activity.

24. The method of claim 17, 18, 19 or 20 wherein the mutant Family B DNA polymerase comprises an amino acid mutation at the amino acids corresponding to L409 to P411 of SEQ ID NO:3

25. The method of claim 17, 18, 19 or 20 wherein the mutant Family B DNA polymerase comprises an amino acid mutation at the position corresponding to L409 of SEQ ID NO: 3.

26. The method of claim 25, wherein the amino acid mutation at the amino acid corresponding to L409 of SEQ ID NO: 3 is a leucine to phenylalanine mutation, leucine to tyrosine mutation, leucine to histidine mutation or a leucine to tryptophan mutation.

27. The method of claim 17, wherein the first and second reaction mixtures occur in the same reaction tube.

28. The method of claim 17, wherein the second reaction mixture comprises a second DNA polymerase or a combination of two or more other DNA polymerases.

29. The method of claim 28, wherein the second DNA polymerase is a wild-type DNA polymerase.

30. The method of claim 28, wherein the second DNA polymerase comprises E. coli DNA polymerase I, Klenow, Exo− Pfu V93, Exo− Pfu or Pfu DNA polymerase.

31. The method of claim 17, wherein the transcribing step incorporates a non-conventional nucleotide into the anti-sense RNA.

32. The method of claim 17, further comprising a coupling step.

33. The method of claim 32, wherein the coupling step comprising coupling the anti-sense RNA to a fluorescent dye containing a NHS- or STP-ester leaving group.

34. The method of claim 17, wherein the primer complex contains a non-conventional conventional nucleotide.

35. A method for amplifying an RNA molecule, comprising:
incubating a template RNA molecule with a first primer complex in a first reaction mixture comprising a mutant Family B DNA polymerase exhibiting an increased reverse transcriptase activity, wherein the first primer complex comprises a primer complementary to the template and a promoter region and wherein the incubation permits synthesis of a complementary DNA template;
incubating the complementary DNA template and a second primer complex in a second reaction mixture, wherein the second reaction mixture permits synthesis of a second complementary DNA containing the promoter region; and
transcribing copies of RNA initiated from the promoter region of the primer complex, wherein the transcription generates synthesized RNA.


SEQUENCE LISTING (106 sequences)

SEQ ID NO: 1 - protein, 776 aa, Thermococcus sp. JDF-3
MILDVDYITENGKPVIRVFKKENGEFRIEYDREFEPYFYALLRDDSAIEEIKKITAERHGRVVKVKRAEKVKKKFLGRSVEVWVLYFTHPQDVPAIRDKIRKHPAVIDIYEYDIPFAKRYLIDKGLIPMEGEEELKLMSFDIETLYHEGEEFGTGPILMISYADESEARVITWKKIDLPYVEVVSTEKEMIKRFLRVVKEKDPDVLITYNGDNFDFAYLKKRCEKLGVSFTLGRDGSEPKIQRMGDRFAVEVKGRVHFDLYPVIRRTINLPTYTLEAVYEAVFGKPKEKVYAEEIATAWETGEGLERVARYSMEDARVTYELGREFFPMEAQLSRLIGQGLWDVSRSSTGNLVEWFLLRKAYERNELAPNKPDERELARRRGGYAGGYVKEPERGLWDNIVYLDFRSLYPSIIITHNVSPDTLNREGCRSYDVAPEVGHKFCKDFPGFIPSLLGNLLEERQKIKRKMKATLDPLEKNLLDYRQRAIKILANSYYGYYGYARARWYCRECAESVTAWGREYIEMVIRELEEKFGFKVLYADTDGLHATIPGADAETVKKKAMEFLNYINPKLPGLLELEYEGFYVRGFFVTKKKYAVIDEEGKITTRGLEIVRRDWSEIAKETQARVLEAILRHGDVEEAVRIVREVTEKLSKYEVPPEKLVIHEQITRELKDYKATGPHVAIAKRLAARGVKIRPGTVISYIVLKGSGRIGDRAIPFDEFDPTKHKYDADYYIENQVLPAVERILRAFGYRKEDLRYQKTRQVGLGAWLKPKGKKK

SEQ ID NO: 2 - DNA, 5255 bp, Thermococcus sp. JDF-3
  misc_feature (3518)..(3519): n is a, c, g, or t
aattccactgccgtgtttaacctttccaccgttgaacttgagggtgattttctgagcctcctcaatcacttaatcgagaccgcggattaccttgaactggtacacgttcaacgattcggttcttgtaatggtcgatactgggccgtgctggattttctaaacgtctcaagaacggctttcatcaacggaaactgccacgtctccgccgtcgtgagggttaaacctgaagttcaagactttgcaacggaatggcgagagaacggcgactaccccagtggaagagcttttgaaagccaaagccgagcttcagcgaatgtgcggtgcccttgttcaagagttgtgagcccttgattgttgttttctcctcttttctgataacatcgatggcgaagtttattagttctcagttcgataatcaggcaggtgttggtcatgatccttgacgttgattacatcaccgagaatggaaagcccgtcatcagggtcttcaagaaggagaacggcgagttcaggattgaatacgaccgcgagttcgagccctacttctacgcgctcctcagggacgactctgccatcgaagaaatcaaaaagataaccgcggagaggcacggcagggtcgttaaggttaagcgcgcggagaaggtgaagaaaaagttcctcggcaggtctgtggaggtctgggtcctctacttcacgcacccgcaggacgttccggcaatccgcgacaaaataaggaagcaccccgcggtcatcgacatctacgagtacgacatacccttcgccaagcgctacctcatagacaagggcctaatcccgatggaaggtgaggaagagcttaaactcatgtccttcgacatcgagacgctctaccacgagggagaagagtttggaaccgggccgattctgatgataagctacgccgatgaaagcgaggcgcgcgtgataacctggaagaagatcgaccttccttacgttgaggttgtctccaccgagaaggagatgattaagcgcttcttgagggtcgttaaggagaaggacccggacgtgctgataacatacaacggcgacaacttcgacttcgcctacctgaaaaagcgctgtgagaagcttggcgtgagctttaccctcgggagggacgggagcgagccgaagatacagcgcatgggggacaggtttgcggtcgaggtgaagggcagggtacacttcgacctttatccagtcataaggcgcaccataaacctcccgacctacacccttgaggctgtatacgaggcggttttcggcaagcccaaggagaaggtctacgccgaggagatagccaccgcctgggagaccggcgaggggcttgagagggtcgcgcgctactcgatggaggacgcgagggttacctacgagcttggcagggagttcttcccgatggaggcccagctttccaggctcatcggccaaggcctctgggacgtttcccgctccagcaccggcaacctcgtcgagtggttcctcctaaggaaggcctacgagaggaacgaactcgctcccaacaagcccgacgagagggagctggcgaggagaagggggggctacgccggtggctacgtcaaggagccggagcggggactgtgggacaatatcgtgtatctagactttcgtagtctctacccttcaatcataatcacccacaacgtctcgccagatacgctcaaccgcgaggggtgtaggagctacgacgttgcccccgaggtcggtcacaagttctgcaaggacttccccggcttcattccgagcctgctcggaaacctgctggaggaaaggcagaagataaagaggaagatgaaggcaactctcgacccgctggagaagaatctcctcgattacaggcaacgcgccatcaagattctcgccaacagccttcttcccggggagtgggttgcggtcattgaaggggggaaactcaggcccgtccgcatcggcgagctggttgatggactgatggaagccagcggggagagggtgaaaagagacggcgacaccgaggtccttgaagtcgaggggctttacgcctctccttcgacagggagtccaagaaagcccgcacaatgccggtgaaagccgtgataaggcaccgctatgccggggaagtttacagaatagctctcaactccggaaggaggattaagcgtgacgcgcggccacagcctcttcgcgtaccgggacgcgagcttgtggaggtgacgggggaggaggaggttcaagcccggcgacctcctggcggtgccaagcggataaccctcccggagaggagggagaggctcaacatcgttgaactgctcctcgaactgcccgaggaggaaacggccgacatgtcatcgacattccggcaagggtagaaagaacttcttcaggggaatgctcagaaccctccgctggattttcggggaggagaagaccggagggcggccaggcgctacctggagcaccttgcgtgggctcggctacgtgaagctgaggaaaatcggctacggggtggttgatagggagggactgggaaaggtaccgcgcttctacgagaggctcgtggaggtaatccgctacaacggcaacaggggggagttcatcgccgatttcaacgcgctccgccccgtcctccgcctgatgatgcccgagaaggagcttgaagagtggctcgttgggacgaggaacgggttcaggataaggccgttcatagaggttgattggaagttcgcaaagctcctcggctactacgtgagcgaggggagcgccgggaagtggaaaaaccggaccgggggctggagctactcggtgaggctttacaacgaggacgggagcgttctcgacgacatggagagactcgcgaggagttctttgggggcgtgagcgcggggggaactacgtcgagatttcaaagaagatggcctacataatcttcgaggggctctgcggttcaccggccgagaacaagagggttccgtggcttatcttcacgtcccctgaggaggtccgctgggccttccttgaggggtacttcatcggcgacggcgacgttcacccgagcaagatggttcggctctccaccaagagcgagcttctggctaacggcctcgtcctgctcctgaactcgctgggcgtctcagcgataaacgtccgccacgacagcggggtttacagggtctacgtgaacgaggaactgccctttacagagtaccggaagcggaagaacgcctcacttactcccacgtcataccgagggaagtgctggaggagacttcggccgggccttccagaagaacatgagtcacgggaaattcagggagctggtggaaagcggggagctcgacgcggaaagggccggtaggataggctggctcctcgacggggatatagtcctcgacagggtctcggaagtcaggaaggaaagctacgaggggtacgtctacgacctgagcgttgaggaggacgagaacttctggcgggctttgggttcctctacgcgcacaacnnagctactacggctactacggctatgccagggcaagatggtactgcagggagtgcgccgagagcgttacggcatggggaagggagtacatcgaaatggtcatcagagagcttgaggaaaagttcggttttaaagtcctctatgcagacacagacggtctccatgccaccattcctggagcggacgctgaaacagtcaagaaaaaggcaatggagttcttaaactatatcaatcccaaactgcccggccttctcgaactcgaatacgagggcttctacgtcaggggcttcttcgtcacgaagaaaaagtacgcggtcatcgacgaggagggcaagataaccacgcgcgggcttgagatagtcaggcgcgactggagcgagatagcgaaggagacgcaggcgagggttttggaggcgatactcaggcacggtgacgttgaagaggccgtcagaattgtcagggaagtcaccgaaaagctgagcaagtacgaggttccgccggagaagctggttatccacgagcagataacgcgcgagctcaaggactacaaggccaccggcccgcacgtagccatagcgaagcgtttggccgccagaggtgttaaaatccggcccggaactgtgataagctacatcgttctgaagggctccggaaggataggcgacagggcgattcccttcgacgagttcgacccgacgaagcacaagtacgatgcggactactacatcgagaaccaggttctgccggcagttgagagaatcctcagggccttcggctaccgcaaggaagacctgcgctaccagaagacgaggcaggtcgggcttggcgcgtggctgaagccgaaggggaagaagaagtgaggaattatctggtttcttttcccagcattaaatgcttccgacattgccttatttatgaaactcctgttgtgcctgagtttgtgccagaaaacagcctgttctgacggcgctttttcttgccaggtctcttgagtttcgcaagggtcttctcgaccagctcaatggtcttgtcgtcattgtttnnnnnnnnnnnnnnnnnnnnncccggggacttcatactggcggtaatagacagggattccttcctcaaggacttcccgggaggcattggagttttttggtggggctttcacaggatttgctcatcttgtggatttctcgttcgattgaatctgtccacttgagggtgtaggtcgagacggtggagcgcgtattccgggagcgggtcttgaggctccatttttcagtcctcctccggcgaagaagtggaactcaagccgggtgttagcttatgttatgttcccaactcctccagcacctccaggatcccctcaatcccggaacctcgaagcccctctcgtggatctttctaacttcctctgcctccgggtttatccagaccgcccacatgccggctctcagcgcaccctcgaaatcctccgcgtaggtgtcgccgatgtggattgcctcgtccggctcgaccccgaagcatcgagcggttttctgaacatctcgggcatcggcttatacgccagaacctcgtcggcgaagaaggttccctcaatgtagtccatcaggccgaacctctcgagggggggcccggtacccaattcgccctatagtgagtcgattacaattcactggccgtcgttttacaacgtcgtgactgggaaaaccctggcgttacccaacttaagtcgctttgcagcacatccccc

SEQ ID NO: 3 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 4 - DNA, 2328 bp, Pyrococcus furiosus
atgattttagatgtggattacataactgaagaaggaaaacctgttattaggctattcaaaaaagagaacggaaaatttaagatagagcatgatagaacttttagaccatacatttacgctcttctcagggatgattcaaagattgaagaagttaagaaaataacgggggaaaggcatggaaagattgtgagaattgttgatgtagagaaggttgagaaaaagtttctcggcaagcctattaccgtgtggaaactttatttggaacatccccaagatgttcccactattagagaaaaagttagagaacatccagcagttgtggacatcttcgaatacgatattccatttgcaaagagatacctcatcgacaaaggcctaataccaatggagggggaagaagagctaaagattcttgccttcgatatagaaaccctctatcacgaaggagaagagtttggaaaaggcccaattataatgattagttatgcagatgaaaatgaagcaaaggtgattacttggaaaaacatagatcttccatacgttgaggttgtatcaagcgagagagagatgataaagagatttctcaggattatcagggagaaggatcctgacattatagttacttataatggagactcattcgacttcccatatttagcgaaaagggcagaaaaacttgggattaaattaaccattggaagagatggaagcgagcccaagatgcagagaataggcgatatgacggctgtagaagtcaagggaagaatacatttcgacttgtatcatgtaataacaaggacaataaatctcccaacatacacactagaggctgtatatgaagcaatttttggaaagccaaaggagaaggtatacgccgacgagatagcaaaagcctgggaaagtggagagaaccttgagagagttgccaaatactcgatggaagatgcaaaggcaacttatgaactcgggaaagaattccttccaatggaaattcagctttcaagattagttggacaacctttatgggatgtttcaaggtcaagcacagggaaccttgtagagtggttcttacttaggaaagcctacgaaagaaacgaagtagctccaaacaagccaagtgaagaggagtatcaaagaaggctcagggagagctacacaggtggattcgttaaagagccagaaaaggggttgtgggaaaacatagtatacctagattttagagccctatatccctcgattataattacccacaatgtttctcccgatactctaaatcttgagggatgcaagaactatgatatcgctcctcaagtaggccacaagttctgcaaggacatccctggttttataccaagtctcttgggacatttgttagaggaaagacaaaagattaagacaaaaatgaaggaaactcaagatcctatagaaaaaatactccttgactatagacaaaaagcgataaaactcttagcaaattctttctacggatattatggctatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaagaagaggtatgcagtaatagatgaagaaggaaaagtcattactcgtggtttagagatagttaggagagattggagtgaaattgcaaaagaaactcaagctagagttttggagacaatactaaaacacggagatgttgaagaagctgtgagaatagtaaaagaagtaatacaaaagcttgccaattatgaaattccaccagagaagctcgcaatatatgagcagataacaagaccattacatgagtataaggcgataggtcctcacgtagctgttgcaaagaaactagctgctaaaggagttaaaataaagccaggaatggtaattggatacatagtacttagaggcgatggtccaattagcaatagggcaattctagctgaggaatacgatcccaaaaagcacaagtatgacgcagaatattacattgagaaccaggttcttccagcggtacttaggatattggagggatttggatacagaaaggaagacctcagataccaaaagacaagacaagtcggcctaacttcctggcttaacattaaaaaatcctag

SEQ ID NO: 5 - protein, 774 aa, Thermococcus kodakaraensis
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 6 - DNA, 2325 bp, Thermococcus kodakaraensis
atgatcctcgacactgactacataaccgaggatggaaagcctgtcataagaattttcaagaaggaaaacggcgagtttaagattgagtacgaccggacttttgaaccctacttctacgccctcctgaaggacgattctgccattgaggaagtcaagaagataaccgccgagaggcacgggacggttgtaacggttaagcgggttgaaaaggttcagaagaagttcctcgggagaccagttgaggtctggaaactctactttactcatccgcaggacgtcccagcgataagggacaagatacgagagcatccagcagttattgacatctacgagtacgacatacccttcgccaagcgctacctcatagacaagggattagtgccaatggaaggcgacgaggagctgaaaatgctcgccttcgacattgaaactctctaccatgagggcgaggagttcgccgaggggccaatccttatgataagctacgccgacgaggaaggggccagggtgataacttggaagaacgtggatctcccctacgttgacgtcgtctcgacggagagggagatgataaagcgcttcctccgtgttgtgaaggagaaagacccggacgttctcataacctacaacggcgacaacttcgacttcgcctatctgaaaaagcgctgtgaaaagctcggaataaacttcgccctcggaagggatggaagcgagccgaagattcagaggatgggcgacaggtttgccgtcgaagtgaagggacggatacacttcgatctctatcctgtgataagacggacgataaacctgcccacatacacgcttgaggccgtttatgaagccgtcttcggtcagccgaaggagaaggtttacgctgaggaaataaccacagcctgggaaaccggcgagaaccttgagagagtcgcccgctactcgatggaagatgcgaaggtcacatacgagcttgggaaggagttccttccgatggaggcccagctttctcgcttaatcggccagtccctctgggacgtctcccgctccagcactggcaacctcgttgagtggttcctcctcaggaaggcctatgagaggaatgagctggccccgaacaagcccgatgaaaaggagctggccagaagacggcagagctatgaaggaggctatgtaaaagagcccgagagagggttgtgggagaacatagtgtacctagattttagatccctgtacccctcaatcatcatcacccacaacgtctcgccggatacgctcaacagagaaggatgcaaggaatatgacgttgccccacaggtcggccaccgcttctgcaaggacttcccaggatttatcccgagcctgcttggagacctcctagaggagaggcagaagataaagaagaagatgaaggccacgattgacccgatcgagaggaagctcctcgattacaggcagagggccatcaagatcctggcaaacagctactacggttactacggctatgcaagggcgcgctggtactgcaaggagtgtgcagagagcgtaacggcctggggaagggagtacataacgatgaccatcaaggagatagaggaaaagtacggctttaaggtaatctacagcgacaccgacggattttttgccacaatacctggagccgatgctgaaaccgtcaaaaagaaggctatggagttcctcaagtatatcaacgccaaacttccgggcgcgcttgagctcgagtacgagggcttctacaaacgcggcttcttcgtcacgaagaagaagtatgcggtgatagacgaggaaggcaagataacaacgcgcggacttgagattgtgaggcgtgactggagcgagatagcgaaagagacgcaggcgagggttcttgaagctttgctaaaggacggtgacgtcgagaaggccgtgaggatagtcaaagaagttaccgaaaagctgagcaagtacgaggttccgccggagaagctggtgatccacgagcagataacgagggatttaaaggactacaaggcaaccggtccccacgttgccgttgccaagaggttggccgcgagaggagtcaaaatacgccctggaacggtgataagctacatcgtgctcaagggctctgggaggataggcgacagggcgataccgttcgacgagttcgacccgacgaagcacaagtacgacgccgagtactacattgagaaccaggttctcccagccgttgagagaattctgagagccttcggttaccgcaaggaagacctgcgctaccagaagacgagacaggttggtttgagtgcttggctgaagccgaagggaacttga

SEQ ID NO: 7 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 8 - DNA, 2325 bp, Thermococcus litoralis
atgatactggacactgattacataacaaaagatggcaagcctataatccgaatttttaagaaagagaacggggagtttaaaatagaacttgaccctcattttcagccctatatatatgctcttctcaaagatgactccgctattgaggagataaaggcaataaagggcgagagacatggaaaaactgtgagagtgctcgatgcagtgaaagtcaggaaaaaatttttgggaagggaagttgaagtctggaagctcattttcgagcatccccaagacgttccagctatgcggggcaaaataagggaacatccagctgtggttgacatttacgaatatgacataccctttgccaagcgttatctcatagacaagggcttgattcccatggagggagacgaggagcttaagctccttgcctttgatattgaaacgttttatcatgagggagatgaatttggaaagggcgagataataatgattagttatgccgatgaagaagaggccagagtaatcacatggaaaaatatcgatttgccgtatgtcgatgttgtgtccaatgaaagagaaatgataaagcgttttgttcaagttgttaaagaaaaagaccccgatgtgataataacttacaatggggacaattttgatttgccgtatctcataaaacgggcagaaaagctgggagttcggcttgtcttaggaagggacaaagaacatcccgaacccaagattcagaggatgggtgatagttttgctgtggaaatcaagggtagaatccactttgatcttttcccagttgtgcgaaggacgataaacctcccaacgtatacgcttgaggcagtttatgaagcagttttaggaaaaaccaaaagcaaattaggagcagaggaaattgccgctatatgggaaacagaagaaagcatgaaaaaactagcccagtactcaatggaagatgctagggcaacgtatgagctcgggaaggaattcttccccatggaagctgagctggcaaagctgataggtcaaagtgtatgggacgtctcgagatcaagcaccggcaacctcgtggagtggtatcttttaagggtggcatacgcgaggaatgaacttgcaccgaacaaacctgatgaggaagagtataaacggcgcttaagaacaacttacctgggaggatatgtaaaagagccagaaaaaggtttgtgggaaaatatcatttatttggatttccgcagtctgtacccttcaataatagttactcacaacgtatccccagatacccttgaaaaagagggctgtaagaattacgatgttgctccgatagtaggatataggttctgcaaggactttccgggctttattccctccatactcggggacttaattgcaatgaggcaagatataaagaagaaaatgaaatccacaattgacccgatcgaaaagaaaatgctcgattataggcaaagggctattaaattgcttgcaaacagctattacggctatatggggtatcctaaggcaagatggtactcgaaggaatgtgctgaaagcgttaccgcatgggggagacactacatagagatgacgataagagaaatagaggaaaagttcggctttaaggttctttatgcggacactgacggcttttatgccacaatacccggggaaaagcctgaactcattaaaaagaaagccaaggaattcctaaactacataaactccaaacttccaggtctgcttgagcttgagtatgagggcttttacttgagaggattctttgttacaaaaaagcgctatgcagtcatagatgaagagggcaggataacaacaaggggcttggaagtagtaaggagagattggagtgagatagctaaggagactcaggcaaaggttttagaggctatacttaaagagggaagtgttgaaaaagctgtagaagttgttagagatgttgtagagaaaatagcaaaatacagggttccacttgaaaagcttgttatccatgagcagattaccagggatttaaaggactacaaagccattggccctcatgtcgcgatagcaaaaagacttgccgcaagagggataaaagtgaaaccgggcacaataataagctatatcgttctcaaagggagcggaaagataagcgatagggtaattttacttacagaatacgatcctagaaaacacaagtacgatccggactactacatagaaaaccaagttttgccggcagtacttaggatactcgaagcgtttggatacagaaaggaggatttaaggtatcaaagctcaaaacaaaccggcttagatgcatggctcaagaggtag

SEQ ID NO: 9 - protein, 775 aa, Pyrococcus sp.
MILDADYITEDGKPIIRIFKKENGEFKVEYDRNFRPYIYALLKDDSQIDEVRKITAERHGKIVRIIDAEKVRKKFLGRPIEVWRLYFEHPQDVPAIRDKIREHSAVIDIFEYDIPFAKRYLIDKGLIPMEGDEELKLLAFDIETLYHEGEEFAKGPIIMISYADEEEAKVITWKKIDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCREYDVAPEVGHKFCKDFPGFIPSLLKRLLDERQEIKRKMKASKDPIEKKMLDYRQRAIKILANSYYGYYGYAKARWYCKECAESVTAWGREYIEFVRKELEEKFGFKVLYIDTDGLYATIPGAKPEEIKKKALEFVDYINAKLPGLLELEYEGFYVRGFFVTKKKYALIDEEGKIITRGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDLRKHKYDAEYYIENQVLPAVLRILEAFGYRKEDLRWQKTKQTGLTAWLNIKKK

SEQ ID NO: 10 - DNA, 2328 bp, Pyrococcus sp.
atgatacttgacgctgactacatcaccgaggatgggaagccgattataaggattttcaagaaagaaaacggcgagtttaaggttgagtacgacagaaactttagaccttacatttacgctctcctcaaagatgactcgcagattgatgaggttaggaagataaccgccgagaggcatgggaagatagtgagaattatagatgccgaaaaggtaaggaagaagttcctggggaggccgattgaggtatggaggctgtactttgaacaccctcaggacgttcccgcaataagggataagataagagagcattccgcagttattgacatctttgagtacgacattccgttcgcgaagaggtacctaatagacaaaggcctaattccaatggaaggcgatgaagagctcaagttgctcgcatttgacatagaaaccctctatcacgaaggggaggagttcgcgaaggggcccattataatgataagctatgctgatgaggaagaagccaaagtcataacgtggaaaaagatcgatctcccgtacgtcgaggtagtttccagcgagagggagatgataaagcggttcctcaaggtgataagggagaaagatcccgatgttataattacctacaacggcgattctttcgaccttccctatctagttaagagggccgaaaagctcgggataaagctacccctgggaagggacggtagtgagccaaagatgcagaggcttggggatatgacagcggtggagataaagggaaggatacactttgacctctaccacgtgattaggagaacgataaacctcccaacatacaccctcgaggcagtttatgaggcaatcttcggaaagccaaaggagaaagtttacgctcacgagatagctgaggcctgggagactggaaagggactggagagagttgcaaagtattcaatggaggatgcaaaggtaacgtacgagctcggtagggagttcttcccaatggaggcccagctttcaaggttagtcggccagcccctgtgggatgtttctaggtcttcaactggcaacttggtggagtggtacctcctcaggaaggcctacgagaggaatgaattggctccaaacaagccggatgagagggagtacgagagaaggctaagggagagctacgctgggggatacgttaaggagccggagaaagggctctgggaggggttagtttccctagatttcaggagcctgtacccctcgataataatcacccataacgtctcaccggatacgctgaacagggaagggtgtagggaatacgatgtcgccccagaggttgggcacaagttctgcaaggacttcccggggtttatccccagcctgctcaagaggttattggatgaaaggcaagaaataaaaaggaagatgaaagcttctaaagacccaatcgagaagaagatgcttgattacaggcaacgggcaatcaaaatcctggcaaacagctattatgggtattatgggtacgcaaaagcccgttggtactgtaaggagtgcgcagagagcgttacggcctgggggagggaatatatagagttcgtaaggaaggaactggaggaaaagttcgggttcaaagtcttatacatagacacagatggactctacgccacaattcctggggcaaaacccgaggagataaagaagaaagccctagagttcgtagattatataaacgccaagctcccagggctgttggagcttgagtacgagggcttctacgtgagagggttcttcgtgacgaagaagaagtatgcgttgatagatgaggaagggaagataatcactagggggcttgaaatagtcaggagggactggagcgaaatagccaaagaaacccaagcaaaagtcctagaggctatcctaaagcatggcaacgttgaggaggcagtaaagatagttaaggaggtaactgaaaagctgagcaagtacgaaatacctccagaaaagctagttatttacgagcagatcacgaggccccttcacgagtacaaggctataggtccgcacgttgccgtggcaaaaaggttagccgctagaggagtaaaggtgaggcctggcatggtgatagggtacatagtgctgaggggagacgggccaataagcaagagggctatccttgcagaggagttcgatctcaggaagcataagtatgacgctgagtattacatagaaaatcaggttttacctgccgttcttagaatattagaggcctttgggtacaggaaagaagacctcaggtggcagaagactaaacagacaggtcttacggcatggcttaacatcaagaagaagtaa

SEQ ID NO: 11 - protein, 773 aa, Thermococcus gorgonarius
MILDTDYITEDGKPVIRIFKKENGEFTIDYDRNFEPYIYALLKDDSPIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLIIYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYTKARWYYKECAESVTGWGREYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 12 - DNA, 2322 bp, Thermococcus gorgonarius
atgatcctcgatacagactacataactgaggatggaaagcccgtcatcaggatcttcaagaaggagaacggcgagttcaccatagactacgacagaaactttgagccatacatctacgcgctcttgaaggacgactctccgattgaggacgtcaagaagataactgccgagaggcacggcactaccgttagggttgtcagggccgagaaagtgaagaagaagttcctaggcaggccgatagaggtctggaagctctacttcactcacccccaggacgttcccgcaatcagggacaagataaaggagcatcctgccgttgtggacatctacgagtacgacatccccttcgcgaagcgctacctcatagacaaaggcttaatcccgatggagggcgacgaggaacttaagatgctcgccttcgacatcgagacgctctatcacgagggcgaggagttcgccgaagggcctatcctgatgataagctacgccgacgaggaaggggcgcgcgttattacctggaagaatatcgaccttccctatgtcgacgtcgtttccaccgagaaggagatgataaagcgcttcctcaaggtcgtcaaggaaaaggatcccgacgtcctcataatctacaacggcgacaacttcgacttcgcctacctcaagaagcgctccgagaagctcggagtcaagttcatcctcggaagggaagggagcgaaccgaaaatccagcgcatgggcgatcgctttgcggtggaggtcaagggaaggattcacttcgacctctaccccgtcattaggagaacgattaacctccccacttacacccttgaggcagtatatgaagccatctttggacagccgaaggagaaggtctacgctgaggagatagcgcaggcctgggaaacgggcgagggattagaaagggtggcccgctactcgatggaggacgcgaaggtaacctatgaactcggaaaagagttcttccctatggaagcccagctctcgcgcctcgtaggccagagcctctgggatgtatctcgctcgagtaccggaaacctcgtcgagtggtttttgctgaggaaggcctacgagaggaatgaacttgcaccaaacaagccggacgagagggagctggcaagaagaagggagagctacgcgggtggatacgtcaaggagcccgaaaggggactgtgggagaacatcgtgtatctggacttccgctccctgtatccttcgataataatcacccataacgtctcccctgatacactcaacagggagggttgtgaggagtacgacgtggctcctcaggtaggccataagttctgcaaggacttccccggcttcatcccaagcctcctcggagacctcttggaggagagacagaaggtaaagaagaagatgaaggccactatagacccaatcgagaagaaactcctcgattacaggcaacgagcaatcaaaatccttgctaatagcttctacggttactacggctatacaaaggcccgctggtactacaaggagtgcgccgagagcgttaccggttggggcagggagtacatcgagaccacgataagggaaatagaggagaaatttggctttaaagtcctctacgcggacacagatggatttttcgcaacaatacctggagcggacgccgaaaccgtcaaaaagaaggcaaaggagttcctggactacatcaacgccaaactgcccggcctgctcgaactcgaatacgagggcttctacaagcgcggcttcttcgtgacgaagaagaagtacgcggttatagacgaggaggacaagataacgacgcgcgggcttgaaatagttaggcgtgactggagcgagatagcgaaggagacgcaggcgagggttcttgaggcgatactaaagcacggtgacgttgaagaagcggtaaggattgtcaaagaggttacggagaagctgagcaagtacgaggttccaccggagaagctggtcatctacgagcagataacccgcgacctgaaggactacaaggccaccgggccgcatgtggctgttgcaaaacgcctcgccgcaagggggataaaaatccggcccggaacggtcataagctacatcgtgctcaaaggctcgggaaggattggggacagggctataccctttgacgaatttgacccggcaaagcacaagtacgatgcagaatactacatcgagaaccaggttcttccagctgtggagaggattctgagggcctttggttaccgtaaagaagatttaaggtatcagaaaacgcggcaggttggcttgggggcgtggctaaaacctaagacatga

SEQ ID NO: 13 - protein, 1829 aa, Thermococcus strain TY
  misc_feature (1118)..(1118): Xaa can be any naturally occurring amino acid
MILDTDYITKDGKPIIRIFKKENGEFKIELDPHFQPYIYALLKDDSAIDEIKAIKGERHGKIVRVVDAVKVKKKFLGRDVEVWKLIFEHPQDVPALRGKIREHPAVIDIYEYDIPFAKRYLIDKGLIPMEGDEELKLMAFDIETFYHEGDEFGKGEIIMISYADEEEARVITWKNIDLPYVDVVSNEREMIKRFVQIVREKDPDVLITYNGDNFDLPYLIKRAEKLGVTLLLGRDKEHPEPKIHRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYERNELAPNKPDEEEYRRRLRTTYLGGYVKEPERGLWENIAYLDFRCHPADTKVIVKGKGIVNISDVKEGDYILGIDGWQRVKKVWKYHYEGKLININGLKCTPNHKVPVVTENDRQTRIRDSLAKSFLSGKVKGKIITTKLFEKIAEFEKNKPSEEEILKGELSGIILAEGTLLRKDIEYFDSSRGKKRISHQYRVEITIGENEKELLERILYIFDKLFGIRPSVKKKGDTNALKITTAKKAVYLQIEELLKNIESLYAPAVLRGFFERDATVNKIRSTIVVTQGTNNKWKIDIVAKLLDSLGIPYSRYEYKYIENGKELTKHILEITGRDGLILFQTLVGFISSEKNEALEKAIEVREMNRLKNNSFYNLSTFEVSSEYYKGEVYDLTLEGNPYYFANGILTHNSLYPSIIVTHNVSPDTLEREGCKNYDVAPIVGYKFCKDFPGFIPSILGELITMRQEIKKKMKATIDPIEKKMLDYRQRAVKLLANSILPNEWLPIIENGEVKFVKIGEFIDRYMEEQKDKVRTVDNTEVLEVDNIFAFSLNKESKKSEIKKVKALIRHKYKGEAYEVELNSGRKIHITRGHSLFTIRNGKIKEIWGEEVKVGDLIIVPKKVKLNEKEAVINIPELISKLPDEDTADVVMTTPVKGRKNFFKGMLRTLKWIFGEESKRIRTFNRYLFHLEELGFVKLLPRGYEVTDWEGLKRYRQLYEKLVKNLRYNGNKREYLVRFNDIKDSVSCFPRKELEEWKIGTXKGFRXKCILKVDEDFGKFLGYYVSEGYAGAQKNKTGGMSYSVKLYNENPNVLKDMKNIAEKFFGKVRVGKNCVDIPKKMAYLLAKSLCGVTAENKRIPSIIFDSSEPVRWAFLRAYFVGDGDIHPSKRLRLSTKSELLANQLVFLLNSLGVSSIKIGFDSGVYRVYINEDLPFLQTSRQKNTYYPNLIPKEVLEEIFGRKFQKNITFEKFKELADSGKLDKRKVKLLDFLLNGDIVLDRVKNVEKREYEGYVYDLSVEDNENFLVGFGLLYAHNSYYGYMGYPKARWYSKECAESVTAWGRHYIEMTIKEIEEKFGFKVLYADSVTGDTEIIVKRNGRIEFVPIEKLFERVDYRIGEKEYCILEDVEALTLDNRGKLIWKKVPYVMRHRAKKKVYRIWITNSWYIDVTEDHSLIVAEDGLKEARPMEIEGKSLIATKDDLSGVEYIKPHAIEEISYNGYVYDIEVEGTHRFFANGILVHNTDGFYATIPGEKPETIKKKAKEFLKYINSKLPGLLELEYEGFYLRGFFVAKKRYAVIDEEGRITTRGLEVVRRDWSEIAKETQAKVLEAILKEDSVEKAVEIVKDVVEEIAKYQVPLEKLVIHEQITKDLSEYKAIGPHVAIAKRLAAKGIKVRPGTIISYIVLRGSGKISDRVILLSEYDPKKHKYDPDYYIENQVLPAVLRILEAFGYRKEDLKYQSSKQVGLDAWLKK

SEQ ID NO: 14 - DNA, 5640 bp, Thermococcus strain TY
  misc_feature (3408)..(3408): n is a, c, g, or t
gtttaaaaatttggcggaacttttatttaatctgaattccagttatatctggtggtatctatgatattagacactgactacataacaaaggacggtaaacccataattcgaattttcaagaaagagaacggggaatttaaaatagaacttgatccacattttcagccctacatttacgctcttctcaaagatgactccgctattgatgaaataaaagcaataaaaggcgagagacacggaaaaattgtgagagtagtcgatgcagtgaaagtcaagaagaaatttttggggagagatgttgaggtctggaagcttatatttgagcatccccaagacgtcccggccctaaggggcaagataagggaacatccagctgtgattgacatttatgagtacgacataccctttgccaagcgctacctcatagacaagggcttgatccctatggagggcgacgaggagcttaagctaatggccttcgacattgagacgttttaccacgagggagacgagtttgggaagggcgagataataatgataagctacgccgatgaggaagaggcaagggtaattacatggaagaatattgatctgccctacgttgatgttgtatccaacgaaagggagatgataaagcggtttgtgcaaattgtcagggaaaaagacccggatgtcctgataacttacaatggagacaactttgatttgccgtaccttataaaaagggcagagaagttaggagttactcttctcttggggagggacaaagaacaccccgagcccaagattcacagaatgggcgatagctttgccgtggaaattaaaggcagaattcactttgatctcttcccggttgtgcggagaaccataaacctcccaacatacacgcttgaggcagtttatgaagccgtcttgggaaaaaccaaaagcaagctgggtgcggaggaaatcgccgccatctgggaaacagaggagagcatgaagaagctggcccagtactcgatggaagatgctagggcaacttatgaactcggaaaagagtttttccccatggaggcagagctagcaaagctaataggccaaagcgtatgggacgtctcaagatcaagcactggcaaccttgtagagtggtacctgttaagggtggcatatgagaggaatgagctcgctccgaacaagccggatgaagaagagtacagaaggcgtttaaggactacttacctgggaggatacgtaaaagagccggaaagaggcttatgggagaacatcgcctatttagactttaggtgccaccctgcagatacaaaagtcatagtaaaaggcaaaggaatcgtgaatattagcgatgttaaagaaggagactacatactaggaatagatgggtggcaaagagtcaaaaaagtctggaagtatcattacgaaggcaaactaatcaatataaacggcttaaaatgcactccaaatcataaggtcccggtagtcacggaaaatgatcggcaaactaggattagagatagtcttgcaaaatcattcctctcaggaaaagtaaaaggtaaaattatcacaacaaagctttttgaaaaaattgccgagtttgagaaaaacaagccttctgaagaggaaatattaaagggggaactttcagggattattttagccgagggcacattgcttcgaaaagatattgaatacttcgattcatcgagaggtaaaaagagaatctcacaccaatatagagtcgaaataacaatcggcgaaaacgaaaaggagcttttagagaggattctatacattttcgataagctgtttggcataagaccttctgtgaaaaagaagggagacacaaatgcgcttaaaattactaccgcaaagaaagcggtttaccttcaaatcgaggaacttttgaaaaacattgaaagtctttacgccccagcagtattgaggggcttttttgaaagagatgccactgtgaacaaaatacggagtacaatagttgttactcaaggtacaaacaacaaatggaaaatagatatagtagcaaagttacttgacagccttggaatcccatatagccgctatgagtacaaatacattgaaaatggaaaggagttgactaagcatattttagaaataacaggaagggatggacttattttattccaaacactggtggggttcataagttcagagaaaaacgaagctttagagaaagcaattgaagttagagaaatgaatagacttaagaataacagcttctacaacctttcaacgtttgaagtgtcttctgaatattataaaggcgaggtttatgacctaaccctagaaggaaacccctattattttgcaaatggaattctgactcacaattctctatacccctcaattatagttacccacaacgtctcccctgacactttagaaagagaaggctgcaagaattacgatgttgccccgatagtaggttataagttctgcaaggattttcccggtttcattccatctatactcggggaattaatcacaatgaggcaagaaataaagaagaagatgaaagctacaattgacccaatagaaaagaaaatgcttgattataggcaaagagctgttaaattgctcgcaaacagcattttgccaaatgaatggctaccaataattgaaaacggagaagtaaaattcgtaaaaattggtgagttcatagaccgctacatggaagagcaaaaggacaaagtaagaacagtggataatactgaagttctcgaagtggataacatctttgcattttcactaaataaggaaagcaaaaagagcgagatcaagaaagtcaaagccctcataaggcataagtacaagggtgaagcttacgaggtcgagctcaactctggaagaaaaatccacataacccgtggtcacagcttgttcacgattaggaacgggaaaataaaggaaatatggggagaagaagtaaaagttggcgaccttataatagtgcccaaaaaagttaagctcaatgaaaaagaagccgtcatcaacattccagagttgatttcgaaactgcccgatgaagacacggccgatgttgttatgacaaccccagttaagggaagaaagaacttctttaaaggcatgctgagaacactaaagtggatttttggagaggaaagcaagagaattaggacgtttaacaggtacctcttccatcttgaagagcttggttttgttaaattgctgccccgtggatatgaagtcaccgactgggaagggctaaaaagatataggcaactttacgagaagctcgtaaaaaacctcagatacaatggaaataagagggagtatttagtgaggtttaacgatatcaaagactccgtatcttgcttcccacgaaaggagcttgaagaatggaaaattggnacgntcaaggggtttaggntgaagtgcatcctcaaagtcgatgaggatttcggaaagttcttaggctactatgtcagcgaaggctatgcgggggctcagaaaaacaaaacaggtggaatgagctactcagtaaagctctataatgagaatcctaacgttctcaaagatatgaaaaatattgcagagaagttttttggcaaagttagagtcggcaaaaactgtgtggatataccaaaaaagatggcataccttctcgcgaaatccctctgtggagtaacagcagagaacaaaaggattccttcaattatatttgattcctctgaacctgtacgatgggcattcttgagagcgtattttgtgggtgatggcgatattcatccatcaaaaaggcttaggctctccacaaaaagcgagctcctagcaaaccagcttgtatttctcttaaactccttgggagtttcatctataaaaattgggtttgacagcggggtttatagggtttacataaacgaggatttgccgttcctacaaacttcgaggcagaaaaacacgtactaccctaatcttattcccaaggaggttcttgaggaaatattcggaagaaaattccaaaagaacataacgtttgagaaattcaaagaactcgctgactccggaaaactggacaaaagaaaagtcaagctcttggatttcctcctcaatggagacattgtccttgacagagtaaaaaatgttgaaaaaagagaatatgaagggtatgtctatgatttgagcgttgaagacaacgaaaactttctcgttggttttggactgctctacgcacacaacagctattacggttatatgggctatcccaaggcgaggtggtactcgaaggaatgtgccgaaagcgttaccgcgtggggaaggcactacatagaaatgaccataaaagagatagaggagaaatttggatttaaggtgctatatgccgacagcgttacgggtgatacggagataatcgttaagagaaatgggcggatagagtttgttcctattgaaaagctctttgaacgcgtagattaccggattggagaaaaagaatactgcatccttgaggacgttgaagcgctgacactcgacaatagaggcaagctcatctggaaaaaagttccgtacgttatgaggcacagagcgaaaaagaaggtctaccgcatctggattacaaactcgtggtacattgacgttaccgaggatcactctcttatagtagctgaggatggtctgaaggaagctaggccaatggaaatcgaaggcaagagcctaatagcgaccaaagatgacctttcaggagtggaatatatcaagcctcatgccatcgaggaaatatcctacaacggctacgtttacgacatcgaggtggaggggacccacaggttcttcgcaaatggaatactcgtgcacaacactgatggtttttacgccacaataccgggagaaaaacctgaaacaatcaaaaagaaagctaaggaattcttaaaatacataaactccaaacttcccggtctgctcgagcttgagtatgagggcttttacttgagaggatttttcgtcgcaaagaagcgctatgcggttatagacgaagaaggtaggataacgacaaggggtctggaagttgtaaggagggactggagcgaaatagccaaagagacccaggctaaagtcttggaggcaatacttaaagaagatagtgtcgaaaaagctgtggaaatcgttaaggacgttgttgaggagatagcaaaataccaagtcccgcttgaaaagcttgttatccacgagcagattaccaaggatctaagtgaatacaaagccattgggcctcatgtagcaatagcaaagaggcttgctgcaaagggaataaaagtgagacccggcacgataataagctatatcgtcctcaggggaagcggaaagataagtgacagggtaattttgctttcagagtatgatccgaaaaaacacaagtacgaccccgactactacatagaaaaccaagttctgccggcggtgcttaggatccttgaagccttcggctacagaaaagaggacttaaaataccaaagctcaaaacaggttggactggacgcgtggcttaagaagtagtcttgtctccttttaacccaagcttctctgcgagcttctctatttcttccctgtattctgagatgtggttctcgttaactattagataat

SEQ ID NO: 15 - protein, 775 aa, 9oN Thermococcus sp.
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 16 - DNA, 2408 bp, 9oN Thermococcus sp.
tccagagttgttttacgaggttatgggtggtgagttctcatgattctcgataccgactacatcaccgagaacgggaagcccgtgataagggtcttcaagaaggagaacggcgagtttaaaatcgagtacgacagaaccttcgagccctacttctacgcccttctgaaggacgattctgcgatagaggacgtcaagaaggtaaccgcaaagaggcacggaacggttgtcaaggtgaagcgcgccgagaaggtgcagaagaagttcctcggcaggccgatagaggtctggaagctctacttcaaccatcctcaggacgtcccggcgattcgagacaggatacgcgcccaccccgctgtcgttgacatctacgagtacgacatacccttcgccaagcgctacctcatcgacaagggcctgattccgatggagggcgacgaggagcttacgatgctcgccttcgacatcgaaacgctctatcacgagggcgaggagttcggaaccgggccgattctcatgataagctacgccgacgggagcgaggcgagggtgataacctggaagaagattgaccttccgtacgttgacgtcgtctcgaccgagaaggagatgattaagcgcttcctccgcgtcgtcagggagaaggaccccgacgtgctcatcacctacaacggcgacaacttcgacttcgcctacctgaagaagcgctgtgaggaactcggaataaagttcacactcggcagggacgggagcgagccgaagatacagcgaatgggcgaccgctttgccgttgaggtgaagggcaggattcacttcgacctctaccccgtcataaggcgcacgataaacctcccgacctacacccttgaggccgtttacgaggccgtctttggaaagcccaaggagaaggtttacgcagaggagatagcgcaggcctgggagagcggggagggccttgaaagggttgcaagatactcgatggaggacgctaaggtgacctacgagctgggaagggagttcttcccgatggaggcccagctttcgaggcttataggccagagcctctgggacgtctcgcgctcgagcaccggaaatttggtggagtggttcctcctgcggaaggcctacaagaggaacgagctcgccccaaacaagcccgacgagagggagctcgcgagacggcgcgggggctacgctggcgggtacgttaaggaaccagagcggggattgtgggacaacattgtgtatttagacttccgctcgctgtatccttcaatcatcataacccacaacgtctcgccggataccctcaaccgcgagggctgtaaagagtacgacgtcgcccctgaggttggacacaagttctgcaaggacttccccggcttcataccaagcctcctgggagatttgctcgaggagaggcagaagataaagcggaagatgaaggcaacggttgacccgctggagaagaaactcctcgattacaggcagagggctatcaaaatcctcgccaacagcttctacggctactacggctacgccaaggcccggtggtactgcaaggagtgcgccgagagcgttacggcctggggaagggagtatatagaaatggttatccgggaactcgaagaaaaattcggttttaaagttctctatgccgatacagacggtctccatgctaccattcccggagcagacgctgaaacagtcaagaaaaaagcaaaggagttcttaaaatacattaatccaaaactgcccggcctgctcgaacttgagtacgagggcttctacgtgaggggcttcttcgtcacgaagaagaagtacgctgtgatagacgaggagggcaagataaccacgaggggtcttgagattgtgaggcgcgactggagcgagatagcgaaggagacccaggccagggtcttagaggcgatactcaagcacggtgacgtcgaggaggccgttaggatagtcaaggaagtgacggaaaagctgagcaagtatgaggtcccgcccgagaagctggtaatccacgagcagataacgcgcgatttgagggattacaaagccaccggcccgcacgttgccgttgcgaagaggctcgcggcgcgtggagtgaaaatccggcccggcacggtgataagctacatcgtcctaaagggctctggaaggataggcgacagggcgattccagctgatgagttcgacccgacgaagcaccgctacgatgcggaatactacatcgagaaccaggttctcccggcggtggagaggattctaaaagccttcggctatcggaaggaggatttgcgctaccagaagacgaagcaggtcggcttgggcgcgtggctgaaggtgaaggggaagaagtgaggaaaacccttttacccttgcaatacaaattgtatttcgag

SEQ ID NO: 17 - protein, 586 aa, Methanobacterium thermoautotrophicum
MEDYRMVLLDIDYVTVDEVPVIRLFGKDKSGGNEPIIAHDRSFRPYIYAIPTDLDECLRELEELELEKLEVKEMRDLGRPTEVIRIEFRHPQDVPKIRDRIRDLESVRDIREHDIPFYRRYLIDKSIVPMEELEFQGVEVDSAPSVTTDVRTVEVTGRVQSTGSGAHGLDILSFDIEVRNPHGMPDPEKDEIVMIGVAGNMGYESVISTAGDHLDFVEVVEDERELLERFAEIVIDKKPDILVGYNSDNFDFPYITRRAAILGAELDLGWDGSKIRTMRRGFANATAIKGTVHVDLYPVMRRYMNLDRYTLERVYQELFGEEKIDLPGDRLWEYWDRDELRDELFRYSLDDVVATHRIAEKILPLNLELTRLVGQPLFDISRMATGQQAEWFLVRKAYQYGELVPNKPSQSDFSSRRGRRAVGGYVKEPEKGLHENIVQFDFRSLYPSIIISKNISPDTLTDDEESECYVAPEYGYRFRKSPRGFVPSVIGEILSERVRIKEEMKGSDDPMERKILNVQQEALKRLANTMYGVYGYSRFRWYSMECAEAITAWGRDYIKKTIKTAEEFGFHTVYADTDGFYATYRG

SEQ ID NO: 18 - DNA, 1761 bp, Methanobacterium thermoautotrophicum
atggaagattacagaatggtcctcctcgacatagactacgtgaccgtcgatgaggtcccggttatcagacttttcggtaaggataaaagcggcggaaatgagcccattatagcccatgaccgctcattcagaccctacatatacgcaatccccacagacctcgatgaatgcctcagggagcttgaggaactggaactggagaaacttgaagtcaaagagatgagggaccttggaaggcccacagaggtcatcagaatcgaattcagacacccccaggacgtgccaaagatcagggacaggataagggacctggaatctgtcagggatataagggaacatgacatcccattctacaggcgctacctaatagataaatccatagtacccatggaggagctggagttccagggtgttgaggtggactcagcaccatcggtgacaaccgatgtcaggacggtggaggtaaccggcagggtccagagcacaggttcaggggcacatggactcgacatcctgagctttgacatcgaggtgaggaacccccatggcatgcctgaccctgaaaaggacgagatagtgatgattggtgtcgccggtaacatgggctacgaatctgtcatatcaacggccggcgaccaccttgactttgttgaggtggttgaggatgaaagggaactccttgagagattcgcagagatcgtaattgataagaaaccggacatactggtgggatacaattcagacaactttgacttcccctacataacaaggagggcggctatcctgggcgcggaactcgacctcggctgggatggttcaaagatcaggaccatgaggaggggctttgcaaacgccacggccataaagggaacggtgcacgtggacctctacccggtcatgaggaggtacatgaaccttgacaggtacaccctggagagggtctaccaggaactcttcggcgaggagaagatcgacctccccggtgacaggctctgggagtactgggaccgggatgagctgagggatgaactcttcaggtactccctggatgatgttgttgcaacccacaggatagcagagaagatactccccctcaacctggagctcacccggctggtgggccagccactattcgacatctcccgcatggcaacaggtcagcaggcggaatggttccttgtccgcaaggcataccagtacggggaactggtaccaaacaagccatcccagtcagacttctccagcaggaggggtcgcagggcggtcggcggatacgtcaaggaaccagagaagggtctccacgagaacatagtccagtttgacttcaggagcctctacccaagcataataatctcaaagaacatctcaccggacaccctgacagatgacgaggaatctgaatgctacgtggcacctgaatacggttacaggttccgcaaaagtccgaggggattcgtaccctcggttataggtgaaatactctcagagagggtgaggatcaaggaggagatgaagggatcagatgatcccatggagaggaagatactcaatgtgcagcaggaggccctcaagagacttgcaaacaccatgtacggtgtatatgggtattcaaggttccgctggtactcaatggagtgcgccgaggccataaccgcatggggcagggactatatcaagaaaacaataaaaactgcagaggaatttgggttccacacggtctatgccgacaccgatggtttctatgcaacctacaggggatag

SEQ ID NO: 19 - protein, 1087 aa, Thermoplasma acidophilum
MIMSQKPFDLEGYRKYITEKVREAFNVAQEARAKGLDVSDHVEIPLASDMAERIEALIGIKGIAQEIRDLSSRMSREEVSLEMSRRIAAMFKDNRKEALDKAIRVGLAILTEGILVAPLEGIADVYIGKNQDGSEYVGISYAGPIRGAGGTAQALSVLIGDVVRRELGISRFQPTEDEIERYIEEIESYDRIKHLQYMPTPDEIKLVVRNSPICIDGEGSEEEEVSGHRDMERIKTNRIRGGMCLVLCEGLVQKARKILKYTSSMHLDDWNFLANLGGKAEGKSSKKSDKFLKDIVAGRPVFSHPSRPGGFRLRYGRSRVSGLAAASLNPATMYIMGKFIAIGSQIKVELPGKAAAVTPCDTIDGPTVLLKNGDHVKINDIEKAREVYDDVVEITDAGEILIAYGDFLENNYPLPTPSFTVEWWEQYLPDGVNAKDIDQFSAVEISRKYGIPLHPYYDYYWHDISFEDLEFLVKNAEQWSITEDGMRVPYPAFDVFIRLGIEFRRSGDYLIIRDYYPLLISLGYDVRNGKIVNVKKYERKGSVMETVNYLSGLIIKPRAPTRVGSRLGRPEKAGDRKMKPMVHSLFPVESYGEARRSIIGANKNSEGSYKAEVFFYRCNSCGFETPTPVCPRCGGHCSPLGEKTGSIDLESILNRAESILGISLDSLKEFKGVKKLMSKEKVAEPIEKGILRAVHDISVNKDGTCRFDMSDIPITHFRYREIGIDERTLADLGYEVRDVNELFPQDVIIPRKAAKYLFNVSRFIDDLLVKYYNMPPFYSLESEEDLIGHLIIGLAPHTSGGVVGRIIGFSDVNAFYAHPFFHAAKRRNCDGDEDSVMLLMDGFLNFSARYLPSTRGGLMDAPLVLSVLINPDEIDKEALNVDTLSRYPVLFYEAAERHASPAEIEDTMMTMKVRIKKTGTYMGSSYTMDTSDINSGVLVSSYKTLGTMDEKINEQLGLAKKLRAVDADDVAARVISTHFLPDMYGNFRKFFSQEFRCTKCNAKYRRIPLSGRCQKCGSTSLTLTIHKGSVVKYLNETLKIAENYRLPDYLKARIDNLARTIKETFPDTEEEEKPEPREVKITGLDMY

SEQ ID NO: 20 - DNA, 3258 bp, Thermoplasma acidophilum
atgtcccagaagccttttgatcttgagggatacaggaaatacataaccgagaaggtcagagaggccttcaatgtggcccaagaggccagggccaagggcctggatgtctccgatcatgtggagatcccgctagcttcggatatggcggagagaatagaggcgctgatcggaataaaggggatagcgcaggaaataagggatctcagcagcaggatgtccagggaggaggtatctctggaaatgtccagaaggatagccgccatgttcaaggataacaggaaagaggctctggacaaggccatcagggtgggactcgccatactgacggagggtatactcgtcgcgccccttgagggtatagccgatgtgtacatcggcaagaaccaggatgggagcgagtatgttggcatctcatatgccgggccaatccgcggtgcaggcggtactgcacaggcgctgagcgttctcataggggatgtggtcaggagagagctcggcatatcacgctttcagccaacggaggatgagatagagagatacatagaggagattgaaagttacgatcgcatcaagcaccttcagtatatgccgacaccggacgagatcaagctggttgtgaggaactcgcccatatgcatagatggcgagggcagcgaagaggaggaggtgtccgggcacagggatatggagaggataaagaccaacaggataaggggcgggatgtgcctggttctgtgcgaaggcctcgtgcagaaggccaggaagatactcaagtatacaagctcaatgcaccttgatgactggaacttcctggcgaacctcggtgggaaggccgagggaaagtcctcgaagaaatcggacaaattcctgaaggatatagttgcaggcaggccagttttctcacacccatccaggccaggcggtttcaggctcaggtacggaaggagcagggtatctgggcttgccgccgcatcgctaaatcccgcgacaatgtacataatgggcaagttcatagccatcggatctcagataaaggttgagcttcccggaaaggcggctgccgttacaccatgcgatacgatcgatggcccaaccgttttgctgaaaaacggggatcatgtgaagataaacgatatcgagaaggcgcgtgaagtgtacgatgatgttgtggagataaccgatgccggagagatactgatagcctacggcgattttctggagaacaattatccgcttcccacgccctcgttcacggttgaatggtgggagcagtacctgccggatggcgtgaacgcaaaggatatcgatcagttttcagcggtggagatatccaggaagtacggtataccgctgcatccatactacgattattactggcacgatatatcatttgaagatctggaattccttgttaagaatgcggaacagtggagtataaccgaggacggcatgcgggtaccttatccggcctttgacgttttcataagactcggcatcgaattcagaagatctggcgattacctcatcatcagggattactacccgcttcttatctccctgggctacgatgttaggaatgggaagatagtgaacgtgaaaaaatacgaaaggaagggcagcgtgatggaaacggtcaactatctttccggcctcataataaagccgagggcacctaccagggttggctctaggctgggcaggccagagaaggccggggaccggaagatgaaacccatggtgcactcgctgtttcctgtggaaagctacggcgaggccagaagatccatcataggagccaacaaaaacagcgagggaagctacaaagctgaggtattcttctatagatgcaattcatgcggcttcgagacgcctacaccagtgtgcccgaggtgtggagggcactgttcacctctgggtgagaaaaccggcagcatagatctcgaatcaatactgaacagggcagaatcgatccttggcatctccctggatagcctgaaggagttcaagggcgttaagaagctgatgtcaaaggagaaagttgcggagcctatagagaaaggcatactcagggctgtgcatgatatttcagtgaacaaggacggaacatgccgtttcgatatgtcagatataccgataacgcatttcaggtaccgtgagataggcattgatgaaaggacacttgcagatctaggttatgaggtcagggatgtcaatgagctgtttccgcaggacgttataattcccagaaaggcggcaaagtatcttttcaacgtctccagattcatcgacgatcttctggtgaaatactacaacatgccaccgttctactcgcttgaaagcgaagaggacctcatcggccacctcataatagggctggccccgcatacatccggcggtgtcgtgggcaggataataggcttcagcgacgtcaacgccttctatgcacatccgttcttccatgccgcaaagaggaggaactgcgacggtgatgaagacagcgtcatgctcctgatggatggctttctcaacttctctgccaggtacctgccatcgaccagaggggggctcatggatgcgccgcttgtcctgtccgtcctgatcaacccggatgagatagacaaggaagcgcttaacgttgatacgctttccaggtatcctgtgcttttctacgaggcggccgaacgccatgcgtctccggccgagatagaggacacgatgatgaccatgaaggtcaggatcaagaagacaggaacctatatgggatcatcgtatacgatggatacctcggacataaacagcggggtactcgtatcctcgtacaagacgcttggaaccatggatgaaaagataaacgagcagcttggccttgcgaagaagctccgtgccgtggacgctgatgacgttgctgccagggttatatccacgcactttctgcctgacatgtacggaaacttcaggaagttcttctcgcaggaattcagatgcaccaagtgcaatgcaaagtacaggcgtataccgttgtccgggcggtgccagaaatgcggatcaacaagcctgacgcttacgatacacaagggaagtgtggtgaagtacctcaacgaaacgctcaagatagccgagaactacaggcttccagattatctgaaggccaggatcgacaaccttgcaaggaccataaaggagaccttcccggataccgaggaggaggaaaagccagaacccagggaagttaaaataaccggcctcgatatgtactga

SEQ ID NO: 21 - protein, 785 aa, Pyrobaculum islandicum
MELKVWPLDITYAVVGSVPEIRIFGILSSGERVVLIDRSFKPYFYVDCAVCEPAALKTALSRVAPIDDVQIVERRFLGRSKKFLKVIAKIPEDVRKLREAAMSIPRVSGVYEADIRFYMRYMIDMGVVPCSWNVAEVEEGGRLGGIPTYVVSQWYGIDEGFPPSLKVMAFDIEVYNERGSPDPIRDPVVMLAIKTNDGHEEVFEASGKDDRGVVRAFVDFIRSYDPDVIVGYNSNGFDWPYLVERAKAVGVPLKVDRLSNPPQQSVYGHWSIVGRANVDLYNIVEEFPEIKLKTLDRVAEYFGVMKREERVLIPGHKIYEYWKDPNKRPLLKRYVLDDVRSTLGLADKLLPFLIQLSSVSGLPLDQVAAASVGNRVEWMLLRYAYRLGEVAPNREEREYEPYKGAIVLEPKPGMYEDVLVLDFSSMYPNIMMKYNLSPDTYLEPGEPDPPEGVNVAPEVGHRFRRSPPGFVPQVLKSLVELRKAVREEAKKYPPDSPEFKILDERQRALKVMANAIYGYLGWVGARWYKREVAESVTAFARAILKDVIEQARRLGIVVVYGDTDSLFVKKHGDVDKLIKYVEEKYGIDIKVDKDYAKVLFTEAKKRYAGLLRDGRIDIVGFEVVRGDWSELAKDVQLRVIEIILKSRDIVEARHGVIKYIREIIERLKNYKFNIDDLIIWKTLDKELDEYKAYPPHVHAAQILKRHGYRVGKGTTIGYVIVKGGEKVSERALPYILLDDIKKIDIDYYIERQIIPAALRIAEVIGVKESDLKTGRMERSLLDFLS

SEQ ID NO: 22 - DNA, 2358 bp, Pyrobaculum islandicum
gtggaactaaaagtttggcctctcgacattacatatgccgtagttggtagtgttcctgagattaggattttcggtattttaagcagcggtgagcgtgttgtacttatcgatagatcttttaagccgtatttctacgtcgattgtgccgtttgtgaaccagctgctctaaagactgcgttgtcgcgagttgcgccaatagacgatgtacaaatagttgaaaggaggttcctcggaaggtctaagaagtttttgaaagttattgctaaaattcctgaggatgtcaggaaacttagagaagccgcaatgtcgataccaagggtttctggcgtttatgaagcagatattcgtttttacatgcgatatatgatagacatgggggtagtgccctgtagttggaatgtggcagaggtagaagaggggggtagacttggcgggattcccacatacgttgtctctcagtggtacgggattgacgaaggctttcccccttcattgaaagttatggcgtttgatatcgaggtttataacgagcgcggctctcccgatcctatcagagatcccgtggttatgttagccataaagacaaacgatgggcacgaggaggtttttgaagctagtgggaaagacgataggggggttgtgcgcgcttttgtggattttatcagaagttatgaccccgatgtaattgttggctataattccaacggctttgattggccttatttagtagagcgtgcgaaagccgtcggcgtgccgctcaaggttgatagactgagcaatccccctcaacagagcgtgtatggacactggtctatcgttggtagagcaaatgtagatctctataacatcgtggaagagttcccggagattaagctaaagactcttgaccgtgttgctgaatattttggtgtaatgaaaagagaagagagggtgttgatcccaggccacaagatttatgaatactggaaagaccccaataaaagacctctacttaagcggtatgttctagacgacgtacgatccactctaggacttgccgacaagctcctaccgtttttaatacaactgtcttctgtatctgggctaccgctggatcaggtggcagcggcgagcgtgggcaacagggtagagtggatgcttcttaggtacgcgtaccgcctgggcgaggtagctccgaacagagaggagagggagtacgaaccatataagggggccatcgtgcttgagccaaagcccgggatgtacgaagacgtgcttgtacttgacttctcttccatgtaccctaacatcatgatgaagtacaacctatcgccggatacgtacctagagcccggcgagccggatccgcccgagggtgtaaacgtggcccccgaggtggggcataggtttaggagaagcccaccaggcttcgtccctcaggtgttgaagagcttggtggagcttagaaaggcggtaagagaggaggcgaagaaataccccccagattcgccagagtttaagatcttagacgagagacaacgtgccctcaaggttatggccaatgccatttatggatacttgggctgggtgggggcccggtggtataagcgggaggtagccgagtctgtgacggcttttgccagagcgattctaaaagacgttattgaacaagctagaaggttgggcattgtggttgtatatggcgatacagacagcctatttgtcaaaaaacatggagacgtggacaaactgatcaagtacgtggaggagaagtacggcatagacataaaggtggacaaggattacgccaaggtgcttttcacggaggctaagaagaggtacgctggcttattgagagatgggcgtatagatattgtgggattcgaggtcgtgaggggagactggagtgaacttgccaaagacgttcagctaagagttatagagattatacttaagtcaagagatattgtggaagctagacatggcgttataaaatatattagagaaataatagaacgactgaaaaattacaagtttaacattgatgatttaataatatggaaaactctagacaaagagttagacgaatataaggcgtatccgcctcatgttcacgctgcgcaaattctcaagagacacggctatcgagtgggcaaaggcactacgataggctatgtcatagttaaaggaggcgaaaaagtatctgagagggcgttgccgtatatactccttgacgatataaaaaagatagatatcgactactacatagagagacagataataccagcggcgttgagaatagcagaggttataggggtgaaagagtcggatcttaaaacggggagaatggaaaggtcgcttcttgattttctaagttag

SEQ ID NO: 23 - protein, 1634 aa, Methanococcus jannaschii
  misc_feature (382)..(382): Xaa can be any naturally occurring amino acid
MGMSMGKIKIDALIDNTYKTIEDKAVIYLYLINSILKDRDFKPYFYVELHKEKVENEDIEKIKEFLLKNDLLKFVENIEVVKKIILRKEKEVIKIIATHPQKVPKLRKIKECEIVKEIYEHDIPFAKRYLIDNEIIPMTYWDFENKKPVSIEIPKLKSVAFDMEVYNRDTEPNPERDPILMASFWDENGGKVITYKEFNHPNIEVVKNEKELIKKIIETLKEYDVIYTYNGDNFDFPYLKARAKIYGIDINLGKDGEELKIKRGGMEYRSYIPGRVHIDLYPISRRLLKLTKYTLEDVVYNLFGIEKLKIPHTKIVDYWANNDKTLIEYSLQDAKYTYKIGKYFFPLEVMFSRIVNQTPFEITRMSSGQMVEYLLMKRAFKXNMIVPNKPDEEEYRRRVLTTYEGGYVKEPEKGMFEDIISMDFRCHPKGTKVVVKGKGIVNIEDVKEGNYVLGIDGWQKVKKVWKYEYEGELINVNGLKCTPNHKIPLRYKIKHKKINKNDYLVRDIYAKSLLTKFKGEGKLILCKDFETIGNYEKYINDMDEDFILKSELIGILLAEGHLLRRDIEYFDSSRGKKRISHQYRVEITVNEDEKDFIEKIKYIFKKLFNYELYVRRKKGTKAITLGCAKKDIYLKIEEILKNKEKYLPNAILRGFFEGDGYVNTVRRAVVVNQGTNNYDKIKFIASLLDRLGIKYSFYTYSYEERGKKLKRYVIEIFSKGDLIKFSILISFISRRKNNLLNEIIRQKTLYKIGDYGFYDLDDVCVSLESYKGEVYDLTLEGRPYYFANGILTHNSLYPSIIISYNISPDTLDCECCKDVSEKILGHWFCKKKEGLIPKTLRNLIERRINIKRRMKKMAEIGEINEEYNLLDYEQKSLKILANSILPDEYLTIIEEDGIKVVKIGEYIDDLMRKHKDKIKFSGISEILETKNLKTFSFDKITKKCEIKKVKALIRHPYFGKAYKIKLRSGRTIKVTRGHSLFKYENGKIVEVKGDDVRFGDLIVVPKKLTCVDKEVVINIPKRLINADEEEIKDLVITKHKDKAFFVKLKKTLEDIENNKLKVIFDDCILYLKELGLIDYNIIKKINKVDIKILDEEKFKAYKKYFDTVIEHGNFKKGRCNIQYIKIKDYIANIPDKEFEDCEIGAYSGKINALLKLDEKLAKFLGFFVTRGRLKKQKLKGETVYEISVYKSLPEYQKEIAETFKEVFGAGSMVKDKVTMDNKIVYLVLKYIFKCGDKDKKHIPEELFLASESVIKSFLDGFLKAKKNSHKGTSTFMAKDEKYLNQLMILFNLVGIPTRFTPVKNKGYKLTLNPKYGTVKDLMLDEVKEIEAFEYSGYVYDLSVEDNENFLVNNIYAHNSVYGYLAFPRARFYSRECAEIVTYLGRKYILETVKEAEKFGFKVLYIDTDGFYAIWKEKISKEELIKKAMEFVEYINSKLPGTMELEFEGYFKRGIFVTKKRYALIDENGRVTVKGLEFVRRDWSNIAKITQRRVLEALLVEGSIEKAKKIIQDVIKDLREKKIKKEDLIIYTQLTKDPKEYKTTAPHVEIAKKLMREGKRIKVGDIIGYIIVKGTKSISERAKLPEEVDIDDIDVNYYIDNQILPPVLRIMEAVGVSKNELKKEGAQLTLDKFFK

SEQ ID NO: 24 - DNA, 4 bp, Artificial
  Methylated Sequence
gatc

SEQ ID NO: 25 - protein, 9 aa, Artificial
  Consensus sequence defining Region II (Motif A)
DXXXLYPSI

SEQ ID NO: 26 - protein, 9 aa, Artificial
  Consensus sequence defining Region III (Motif B)
KXXXNXXYG

SEQ ID NO: 27 - protein, 9 aa, Artificial
  LYP motif which is part of Region II (Motif A)
DXXSLYPSI

SEQ ID NO: 28 - DNA, 30 bp, Artificial
  Synthetic oligonucleotide for mutagenesis
ctagattttagagccttctatccctcgatt

SEQ ID NO: 29 - DNA, 30 bp, Artificial
  Synthetic oligonucleotide for mutagenesis
aatcgagggatagaaggctctaaaatctag

SEQ ID NO: 30 - DNA, 30 bp, Artificial
  Synthetic oligonucleotide for mutagenesis
ctagattttagagcctactatccctcgatt

SEQ ID NO: 31 - DNA, 30 bp, Artificial
  Synthetic oligonucleotide for mutagenesis
aatcgagggatagtaggctctaaaatctag

SEQ ID NO: 32 - DNA, 36 bp, Artificial
  Synthetic oligonucleotide for mutagenesis
ctagactttcgtagtttctacccttcaatcataatc

SEQ ID NO: 33 - DNA, 36 bp, Artificial
  Synthetic oligonucleotide for mutagenesis
gattatgattgaagggtagaaactacgaaagtctag

SEQ ID NO: 34 - DNA, 36 bp, Artificial
  Synthetic oligonucleotide for mutagenesis
ctagactttcgtagttactacccttcaatcataatc

SEQ ID NO: 35 - DNA, 36 bp, Artificial
  Synthetic oligonucleotide for mutagenesis
gattatgattgaagggtagtaactacgaaagtctag

SEQ ID NO: 36 - DNA, 36 bp, Artificial
  Synthetic oligonucleotide for mutagenesis
ctagactttcgtagttggtacccttcaatcataatc

SEQ ID NO: 37 - DNA, 36 bp, Artificial
  Synthetic oligonucleotide for mutagenesis
gattatgattgaagggtaccaactacgaaagtctag

SEQ ID NO: 38 - DNA, 17 bp, Artificial
  Synthetic oligonucleotides for GAPDH target amplification
cgagccacatcgctcag

SEQ ID NO: 39 - DNA, 21 bp, Artificial
  Synthetic oligonucleotide for GAPDH target amplification
catgtagttgaggtcaatgaa

SEQ ID NO: 40 - protein, 17 aa, Pyrococcus furiosus
LAFDIETLYHEGEEFGK

SEQ ID NO: 41 - protein, 38 aa, Pyrococcus furiosus
SEREMIKRFLRIIREKDPDIIVTYNGDSFDFPYLAKRA

SEQ ID NO: 42 - protein, 55 aa, Pyrococcus furiosus
PTYTLEAVYEAIFGKPKEKVYADEIAKAWESGENLERVAKYSMEDAKATYELGKE

SEQ ID NO: 43 - protein, 63 aa, Pyrococcus furiosus
TGGFVKEPEKGLWENIVYLDFRALYPSIIITHNVSPDTLNLEGCKNYDIAPQVGHKFCKDIPG

SEQ ID NO: 44 - protein, 41 aa, Pyrococcus furiosus
DYRQKAIKLLANSFYGYYGYAKARWYCKECAESVTAWGRKY

SEQ ID NO: 45 - protein, 14 aa, Pyrococcus furiosus
KVLYIDTDGLYATI

SEQ ID NO: 46 - protein, 48 aa, Pyrococcus furiosus
KLPGLLELEYEGFYKRGFFVTKKRYAVIDEEGKVITRGLEIVRRDWSE

SEQ ID NO: 47 - protein, 17 aa, JDF-3
MSFDIETLYHEGEEFGT

SEQ ID NO: 48 - protein, 38 aa, JDF-3
TEKEMIKRFLRVVKEKDPDVLITYNGDNFDFAYLKKRC

SEQ ID NO: 49 - protein, 55 aa, JDF-3
PTYTLEAVYEAVFGKPKEKVYAEEIATAWETGEGLERVARYSMEDARVTYELGRE

SEQ ID NO: 50 - protein, 63 aa, JDF-3
RGGYVKEPERGLWDNIVYLDFRSLYPSIIITHNVSPDTLNREGCRSYDVAPEVGHKFCKDFPG

SEQ ID NO: 51 - protein, 41 aa, JDF-3
DYRQRAIKILANSYYGYYGYARARWYCRECAESVTAWGREY

SEQ ID NO: 52 - protein, 14 aa, JDF-3
KVLYADTDGLHATI

SEQ ID NO: 53 - protein, 48 aa, JDF-3
KLPGLLELEYEGFYVRGFFVTKKKYAVIDEEGKITTRGLEIVRRDWSE

SEQ ID NO: 54 - protein, 17 aa, Thermococcus gorgonarius
LAFDIETLYHEGEEFAE

SEQ ID NO: 55 - protein, 38 aa, Thermococcus gorgonarius
TEKEMIKRFLKVVKEKDPDVLITYNGDNFDFAYLKKRS

SEQ ID NO: 56 - protein, 55 aa, Thermococcus gorgonarius
PTYTLEAVYEAIFGQPKEKVYAEEIAQAWETGEGLERVARYSMEDAKVTYELGKE

SEQ ID NO: 57 - protein, 63 aa, Thermococcus gorgonarius
AGGYVKEPERGLWENIVYLDFRSLYPSIIITHNVSPDTLNREGCEEYDVAPQVGHKFCKDFPG

SEQ ID NO: 58 - protein, 41 aa, Thermococcus gorgonarius
DYRQRAIKILANSFYGYYGYAKARWYCKECAESVTAWGRQY

SEQ ID NO: 59 - protein, 14 aa, Thermococcus gorgonarius
KVLYADTDGFFATI

SEQ ID NO: 60 - protein, 48 aa, Thermococcus gorgonarius
KLPGLLELEYEGFYKRGFFVTKKKYAVIDEEDKITTRGLEIVRRDWSE

SEQ ID NO: 61 - protein, 17 aa, Thermococcus litoralis
LAFDIETFYHEGDEFGK

SEQ ID NO: 62 - protein, 38 aa, Thermococcus litoralis
NEREMIKRFVQVVKEKDPDVIITYNGDNFDLPYLIKRA

SEQ ID NO: 63 - protein, 55 aa, Thermococcus litoralis
PTYTLEAVYEAVLGKTKSKLGAEEIAAIWETEESMKKLAQYSMEDARATYELGKE

SEQ ID NO: 64 - protein, 63 aa, Thermococcus litoralis
LGGYVKEPEKGLWENIIYLDFRSLYPSIIVTHNVSPDTLEKEGCKNYDVAPIVGYRFCKDFPG

SEQ ID NO: 65 - protein, 41 aa, Thermococcus litoralis
DYRQRAIKLLANSYYGYMGYPKARWYSKECAESVTAWGRHY

SEQ ID NO: 66 - protein, 14 aa, Thermococcus litoralis
KVLYADTDGFYATI

SEQ ID NO: 67 - protein, 48 aa, Thermococcus litoralis
KLPGLLELEYEGFYLRGFFVTKKRYAVIDEEGRITTRGLEVVRRDWSE

SEQ ID NO: 68 - protein, 17 aa, Thermococcus sp.
LAFDIETLYHEGEEFAK

SEQ ID NO: 69 - protein, 38 aa, Thermococcus sp.
SEREMIKRFLKVIREKDPDVIITYNGDSFDFPYLVKRA

SEQ ID NO: 70 - protein, 55 aa, Thermococcus sp.
PTYTLEAVYEAIFGKPKEKVYAHEIAEAWETGKGLERVAKYSMEDAKVTYELGRE

SEQ ID NO: 71 - protein, 63 aa, Thermococcus sp.
AGGYVKEPEKGLWEGLVGKDFRSLYPSIIITHNVSPDTLNREGCREYDVAPEVGHKFCKDFPG

SEQ ID NO: 72 - protein, 41 aa, Thermococcus sp.
DYRQRAIKILANSYYGYYGYAKARWYCKECAESVTAWGREY

SEQ ID NO: 73 - protein, 14 aa, Thermococcus sp.
KVLYIDTDGLYATI

SEQ ID NO: 74 - protein, 48 aa, Thermococcus sp.
KLPGLLELEYEGFYVRGFFVTKKKYALIDEEGKIITRGLEIVRRDWSE

SEQ ID NO: 75 - protein, 17 aa, Methanococcus voltae
YEFDIPFLRRYMIDQDI

SEQ ID NO: 76 - protein, 38 aa, Methanococcus voltae
VKDEKELIQKTIEILKQYDVIYTYNGDNFDFPYLKKRA

SEQ ID NO: 77 - protein, 53 aa, Methanococcus voltae
TKYKLENVVQELFKINKEAVDYGDIPKMWETEDTTLLRYAYEDALYTYKMGNY

SEQ ID NO: 78 - protein, 63 aa, Methanococcus voltae
EGGYVREPLKGIQEDIVSLDFMSLYPSILISHNISPETVIYEEKERENMELGIIPKTLNELLS

SEQ ID NO: 79 - protein, 41 aa, Methanococcus voltae
EHEQKSIKVLANSHYGYLAFPMARWYSDKCAEMVTGLGRKY

SEQ ID NO: 80 - protein, 14 aa, Methanococcus voltae
KVIYADTDGFYAKW

SEQ ID NO: 81 - protein, 48 aa, Methanococcus voltae
ELPEGMELEFEGHFKRGLFVTKKKYALIEDDGHIVVKGLEVVRRDWSN

SEQ ID NO: 82 - protein, 14 aa, Bacteriophage RB69
ANFDIEVTSPDGFP

SEQ ID NO: 83 - protein, 38 aa, Bacteriophage RB69
NEKELLMEYLNFWQQKTPVILTGWNVESFDIPYVYNRI

SEQ ID NO: 84 - protein, 52 aa, Bacteriophage RB69
PSYSLDYISEFELNVGKLGYNGPISKLRESDHERYISYNIIDVYRVLQIDAK

SEQ ID NO: 85 - protein, 63 aa, Bacteriophage RB69
PGAFVKEPIPNRYKYVMSFDLTSLYPSIIRQVNISPETIAGTFKVAPLHDYINAVAERPSDVY

SEQ ID NO: 86 - protein, 41 aa, Bacteriophage RB69
MTAQINRKLLINSLYGALGNVWFRYYDLRNATAITTFGQMA

SEQ ID NO: 87 - protein, 14 aa, Bacteriophage RB69
FVLYGDTDSIYVSA

SEQ ID NO: 88 - protein, 64 aa, Bacteriophage RB69
NKQHLMFMDREAIAGPPLGSKGIGGFWTGKKRYALNVWDMEGTRYAEPKLKIMGLETQKSSTPK

SEQ ID NO: 89 - protein, 13 aa, Bacteriophage T4
ANCDIEVTGDKFP

SEQ ID NO: 90 - protein, 38 aa, Bacteriophage T4
NERDMLMEYINLWEQKRPAIFTGWNIEGFDVPYIMNRV

SEQ ID NO: 91 - protein, 52 aa, Bacteriophage T4
PSFSLESVAQHETKKGKLPYDGPINKLRETNHQRYISYNIIDVESVQAIDKI

SEQ ID NO: 92 - protein, 63 aa, Bacteriophage T4
PGAFVFEPKPIARRYIMSFDLTSLYPSIIRQVNISPETIRGQFKVHPIHEYIAGTAPKPSDEY

SEQ ID NO: 93 - protein, 41 aa, Bacteriophage T4
NTNQLNRKILINSLYGALGNIHFRYYDLRNATAITIFGQVG

SEQ ID NO: 94 - protein, 14 aa, Bacteriophage T4
FIAAGDTDSVYVCV

SEQ ID NO: 95 - protein, 64 aa, Bacteriophage T4
NREHLMHMDREAISCPPLGSKGVGGFWKAKKRYALNVYDMEDKRFAEPHLKIMGMETQQSSTPK

SEQ ID NO: 96 - protein, 18 aa, Escherichia coli
VSIDIETTRHGELYCIGL

SEQ ID NO: 97 - protein, 34 aa, Escherichia coli
LLEKLNAWFANYDPDVIIGWNVVQFDLRMLQKHA

SEQ ID NO: 98 - protein, 57 aa, Escherichia coli
SSFSLETVAQELLGEGKSIDNPWDRMDEIDRRFAEDKPALATYNLKDCELVTQIFHK

SEQ ID NO: 99 - protein, 63 aa, Escherichia coli
PGGYVMDSRPGLYDSVLVLDYKSLYPSIIRTFLIDPVGLVEGMAQPDPEHSTEGFLDAWFSRE

SEQ ID NO: 100 - protein, 41 aa, Escherichia coli
KPLSQALKIIMNAFYGVLGTTACRFFDPRLASSITMRGHQI

SEQ ID NO: 101 - protein, 14 aa, Escherichia coli
DVIYGDTDSTFVWL

SEQ ID NO: 102 - protein, 59 aa, Escherichia coli
RLTSALELEYETHFCRFLMPTIRGADTGSKKRYAGLIQEGDKQRMVFKGLETVRTDWTP

SEQ ID NO: 103 - protein, 1751 aa, Thermococcus sp. JDF-3
  MISC_FEATURE (117)..(117): X=Stop Codon
MSTAVFNLSTVELEGDFLSLLNHLIETADYLELVHVQRFGSCNGRYWAVLDFLNVSRTAFINGNCHVSAVVRVKPEVQDFATEWRENGDYPSGRAFESQSRASANVRCPCSRVVSPXLLFSPLFXXHRWRSLLVLSSIIRQVLVMILDVDYITENGKPVIRVFKKENGEFRIEYDREFEPYFYALLRDDSAIEEIKKITAERHGRVVKVKRAEKVKKKFLGRSVEVWVLYFTHPQDVPAIRDKIRKHPAVIDIYEYDIPFAKRYLIDKGLIPMEGEEELKLMSFDIETLYHEGEEFGTGPILMISYADESEARVITWKKIDLPYVEVVSTEKEMIKRFLRVVKEKDPDVLITYNGDNFDFAYLKKRCEKLGVSFTLGRDGSEPKIQRMGDRFAVEVKGRVHFDLYPVIRRTINLPTYTLEAVYEAVFGKPKEKVYAEEIATAWETGEGLERVARYSMEDARVTYELGREFFPMEAQLSRLIGQGLWDVSRSSTGNLVEWFLLRKAYERNELAPNKPDERELARRRGGYAGGYVKEPERGLWDNIVYLDFRSLYPSIIITHNVSPDTLNREGCRSYDVAPEVGHKFCKDFPGFIPSLLGNLLEERQKIKRKMKATLDPLEKNLLDYRQRAIKILANSLLPGEWVAVIEGGKLRPVRIGELVDGLMEASGERVKRDGDTEVLEVEGLYASPSTGSPRKPAQCRXKPXXGTAMPGKFTEXLSTPEGGLSVTRGHSLFAYRDASLWRXRGRRRFKPGDLLAVPSGXPSRRGGRGSTSLNCSSNCPRRKRPTCHRHSGKGRKNFFRGMLRTLRWIFGEEKTGGRPGATWSTLRGLGYVKLRKIGYGVVDREGLGKVPRFYERLVEVIRYNGNRGEFIADFNALRPVLRLMMPEKELEEWLVGTRNGFRIRPFIEVDWKFAKLLGYYVSEGSAGKWKNRTGGWSYSVRLYNEDGSVLDDMERLARSSLGAXARGELRRDFKEDGLHNLRGALRFTGREQEGSVAYLHVPXGGPLGLPXGVLHRRRRRSPEQDGSALHQERASGXRPRPAPELAGRLSDKRPPRQRGLQGLRERGTALYRVPEAEERLTYSHVIPREVLEETSAGPSRRTXVTGNSGSWWKAGSSTRKGPVGXAGSSTGIXSSTGSRKSGRKATRGTSTTXALRRTRTSGGLWVPLRAQXSYYGYYGYARARWYCRECAESVTAWGREYIEMVIRELEEKFGFKVLYADTDGLHATIPGADAETVKKKAMEFLNYINPKLPGLLELEYEGFYVRGFFVTKKKYAVIDEEGKITTRGLEIVRRDWSEIAKETQARVLEAILRHGDVEEAVRIVREVTEKLSKYEVPPEKLVIHEQITRELKDYKATGPHVAIAKRLAARGVKIRPGTVISYIVLKGSGRIGDRAIPFDEFDPTKHKYDADYYIENQVLPAVERILRAFGYRKEDLRYQKTRQVGLGAWLKPKGKKKXGIIWFLFPALNASDIALFMKLLLCLSLCQKTACSDGAFSCQVSXVSQGSSRPAQWSCRHCXXXXXXXXPGTSYWRXXTGIPSSRTSREALEFFGGAFTGFAHLVDFSFDXICPLEGVGRDGGARIPGAGLEAPFFSPPPAKKWNSSRVLAYVMFPTPPAPPGSPQSRNLEAPLVDLSNFLCLRVYPDRPHAGSQRTLEILRVGVADVDCLVRLDPEASSGFLNISGIGLYARTSSAKKVPSMXSIRPNLSRGGPVPNSPYSESITIHWPSFYNVVTGKTLALPNLSRFAAHP

SEQ ID NO: 104 - DNA, 4 bp, Artificial
  Methylated sequence
natc

SEQ ID NO: 105 - protein, 775 aa, Artificial
  Pfu DNA polymerase mutant (3′-5′Exo+; V93R; L409Y)
MILDVDYITEEGKPVIRLFKKENGKFKIEHDRTFRPYIYALLRDDSKIEEVKKITGERHGKIVRIVDVEKVEKKFLGKPITVWKLYLEHPQDRPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRAYYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 106 - protein, 775 aa, Artificial
  Pfu DNA polymerase mutant (3′-5′ Exo-; V93R; L409Y)
MILDVDYITEEGKPVIRLFKKENGKFKIEHDRTFRPYIYALLRDDSKIEEVKKITGERHGKIVRIVDVEKVEKKFLGKPITVWKLYLEHPQDRPTIREKVREHPAVVDIFEYDIPFAKRYLIDKGLIPMEGEEELKILAFAIATLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRAYYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS